(19) United States
(12) Patent Application Publication
(10) Pub. No.: US 2022/0195502 A1
(43) Pub. Date: Jun. 23, 2022

(54) METHOD FOR DETECTING SPECIFIC NUCLEIC ACIDS IN SAMPLES

(71) Applicant: GenePath Diagnostics Inc., Ann Arbor, MI (US)

(72) Inventors: Nikhil Phadke, Ann Arbor, MI (US); Karthik Ganesan, Ann Arbor, MI (US); Shatakshi Ranade, Ann Arbor, MI (US); Meenal Agarwal, Ann Arbor, MI (US); Siddharth Anand, Ann Arbor, MI (US); Kunal Patil, Ann Arbor, MI (US)

(21) Appl. No.: 17/603,439

(22) PCT Filed: Apr. 23, 2020
(86) PCT No.: PCT/US2020/029622

§ 371 Date: Oct. 13, 2021

(30) Foreign Application Priority Data
Apr. 24, 2019 (IN) 201941016190

(51) Int. Cl. C12Q 1/6827; C12Q 1/6806; C12Q 1/6883

(52) U.S. Cl. CPC C12Q 1/6827; C12Q 2600/154; C12Q 1/6883; C12Q 1/6806

(57) ABSTRACT

Provided are methods for detecting specific nucleotide sequences in samples. Methods include generating, from the specific nucleotide sequences, nucleic acid constructs containing probe-identification sequences and sample identification sequences, pooling the nucleic acid constructs from the samples into a single combined sample, and determining the abundance of the specific nucleotide sequences in the samples by quantifying the probe-identification sequences and sample-identification sequences of the nucleic acid constructs.

### CROSS-REFERENCE TO RELATED APPLICATIONS

This application claims the benefit of priority of Indian Provisional Application No. 201941016190, filed Apr. 24, 2019, the contents of which are incorporated by reference herein in their entirety.

### REFERENCE TO SEQUENCE LISTING

The instant application contains a Sequence Listing which has been submitted electronically in ASCII format and is hereby incorporated by reference in its entirety. Said ASCII copy, created on Apr. 20, 2020, is named 47898-0003WO1_SL.txt and is 78,466 bytes in size.

### FIELD OF THE INVENTION

The invention relates to a method for detecting specific nucleic acids in samples.

### BACKGROUND OF THE INVENTION

Next generation sequencing (NGS) has revolutionized molecular diagnostics with its unprecedented sequencing capacity which has translated into the ability to rapidly sequence large number of targeted genomic regions and accurately detect low depth genomic variants as compared to conventional molecular techniques such as capillary sequencing and quantitative PCR.

In the context of clinical molecular diagnostics, NGS is predominantly used for the detection of small-scale genomic variants (sequence variants or small indels) at multiple genomic loci in a single experiment. With the reducing cost per base of sequencing in recent years, NGS has become a choice of test for analyzing multiple genomic targets with overlapping phenotypes. Nevertheless, a few classes of genetic variations such as copy number variations (CNVs), deletions/duplications (DelDup) and large-genomic rearrangements (LGRs) pose challenges for typical NGS-based diagnostic pipelines. Advances in bioinformatics have ameliorated these challenges to some extent, however, they remain a challenge.

Other limitations of typical NGS pipelines include unequal coverage of the targets, biases during amplification and ambiguously aligned poor quality reads in case of highly homologous nucleotide sequences. In addition, most current NGS pipelines generate a huge amount of data, which requires much computing power and complicated computer algorithms for calculating data, especially when screening for a large number of target sequences—the average coverage of these large NGS panels is typically in the range of 50-300× and panel size (as 1× coverage) can be as high as 12 Megabases (Mb) for clinical exome and 30 Mb for whole exome sequencing. When screening large numbers of samples, multiple NGS analyses are needed.

Given these challenges associated with NGS, there is a need for an improved method for screening for and/or detecting a large number of target nucleic acid sequences, especially if the target sequences need to be evaluated in a large number of subjects.

### SUMMARY OF THE INVENTION

Described herein are methods for detecting specific nucleic acids (target sequences) in samples by generating nucleotide constructs having nested multi-indexed identifiers.

In one aspect, the present disclosure can relate to a method of determining the abundance of each of one or more target nucleotide sequences in each of one or more samples, the method including: (a) generating nucleic acid constructs from the one or more target nucleotide sequences in the more or more samples, each of the nucleic acid constructs including: (i) a probe-identification sequence (PIDS) that identifies the target nucleotide sequence from which the nucleic acid construct is derived; and (ii) a sample identification sequence (SIDS) that identifies the sample from which the nucleic acid construct is derived; (b) pooling the nucleic acid constructs from the one or more samples into a single combined sample; (c) quantifying the PIDS and the SIDS of the nucleic acid constructs, thereby obtaining quantification results; and (d) determining the abundance of each of the one or more target nucleotide sequences for each of the one or more samples based on the quantification results.

In some embodiments, the nucleic acid constructs can be generated by: (a) contacting each of the one or more samples with a first set of target-specific probes (TSP1s) and a second set of target-specific probes (TSP2s) under sufficient conditions and for a sufficient time to allow the TSP1s and TSP2s to hybridize to their target nucleotide sequences, wherein each of the TSP1s includes, from the 5′ end to the 3′ end, a first common adaptor (CA1), a first PIDS (PIDS1) and a first target-specific sequence (TSS1), and wherein each of the TSP2s includes, from the 5′ end to the 3′ end, a second target-specific sequence (TSS2), a second PIDS (PIDS2) and a second common adaptor (CA2); (b) contacting each of the one or more samples containing TSP1s and TSP2 with a ligase under sufficient conditions and for a sufficient time, such that if the TSS1 and TSS2 hybridized to the target nucleotide sequence and the 3′ end of TSS1 and the 5′ end of TSS2 are immediately adjacent to each other, then the TSP1 and TSP2 are ligated by the ligase to form a ligation product (LP); and (c) amplifying by PCR the LPs to produce the nucleic acid constructs, the PCR amplification step including: (i) amplifying the LPs by PCR using a first PCR primer including, from the 5′ end to the 3′ end, a first tethering adaptor (TA1), a first SIDS (SIDS1), and a sequence corresponding to the CA1; and (ii) amplifying the IAs by PCR using a second PCR primer including, from the 5′ end to the 3′ end, a second TA (TA2), a second SIDS (SIDS2), and a sequence corresponding to the CA2, thereby generating the nucleic acid construct.

In some embodiments, the nucleic acid constructs can be generated by: (a) contacting each of the one or more samples with a first set of target-specific probes (TSP1s) and a second set of target-specific probes (TSP2s) under sufficient conditions and for a sufficient time to allow the TSP1s and TSP2s to hybridize to their target nucleotide sequences, wherein each of the TSP1s includes, from the 5′ end to the 3′ end, a first common adaptor (CA1), a first PIDS (PIDS1) and a first target-specific sequence (TSS1), and wherein each of the TSP2s includes, from the 5′ end to the 3′ end, a second target-specific sequence (TSS2), a second PIDS (PIDS2) and a second common adaptor (CA2); (b) contacting each of the one or more samples containing TSP1s and TSP2s with a polymerase and nucleic acids under sufficient condition and for a sufficient time to allow extension of a TSP1 at the 3′ end, if the TSP1 is hybridized to a target nucleotide sequence, (c) contacting each of the one or more samples containing TSP1s and TSP2s with a ligase under sufficient condition and for a sufficient time to allow ligation of a TSP1 with a TSP2 if the 3′ end of the TSP1 is immediately adjacent to the 5′ end of the TSP2; (d) amplifying by PCR the LPs to produce a one or more nucleic acid constructs, the PCR amplification step including: (i) amplifying the LP by PCR using a first PCR primer including a TA1, the SIDS, and a sequence corresponding to the CAL thereby generating a plurality of intermediate amplicons (IAs), each IAs including a TA1; and (ii) amplifying the IAs by PCR using a second PCR primer including a TA2, a sample identification sequence (SIDS), and a sequence corresponding to the CA2, thereby generating the amplicons.

In some embodiments, the nucleic acid constructs can be generated by: (a) amplifying the target nucleotide sequences by PCR using a first primer, the first primer including, from the 5′ end to the 3′ end, a first common adaptor (CA1), a first PIDS (PIDS1), and a first TSS (TSS1), thereby generating first intermediary PCR products (IPP1); (b) amplifying the IPP1 by PCR using a second primer, the second primer including, from the 5′ end to the 3′ end, a second common adaptor (CA2), a second PIDS (PIDS2), and a second TSS (TSS2), thereby generating second intermediary PCR products (IPP2); (c) amplifying the IPP2 by PCR using a third primer, the third primer including, from the 5′ end to the 3′ end, a first Tethering Adapter (TA1), a first SIDS (SIDS1), and a sequence corresponding to CAL thereby generating third intermediary PCR products (IPP3); (d) amplifying the IPP3 by PCR using a fourth primer, the fourth primer including, from the 5′ end to the 3′ end, a second Tethering Adapter (TA2), a second SIDS (SIDS2), and a sequence corresponding to CA2, thereby generating the nucleic acid constructs.

In some embodiments, the nucleic acid constructs can be double-stranded DNA.

In some embodiments, the 5′ ends of the TSP2s can be phosphorylated.

In some embodiments, at least one of the target nucleotide sequences can include a sequence corresponding to a genomic DNA sequence that contains an genetic aberration, the genetic aberration being a single nucleotide polymorphism, insertion, deletion, duplication, rearrangement, truncation, or translocation, as compared to a wild-type genomic DNA sequence.

In some embodiments, at least one of the target nucleotide sequences can include nucleotide sequences having abnormal methylation status as compared to a wild-type DNA sequence.

In some embodiments, the samples can include samples from one or more subjects.

In some embodiments, the samples can include blood, bone marrow, cerebrospinal fluid, pleural fluid, or urine.

In some embodiments, the samples can be from a single subject, obtained at different times.

In some embodiments, the samples can include at least 100 samples, at least 1,000 samples, at least 10,000 samples, at least 100,000 samples, at least 1,000,000 samples, at least 10,000,000 samples, at least 100,000,000 samples, or at least 1,000,000,000 samples.

In some embodiments, the target nucleotide sequences can include at least 100 target nucleotide sequences, at least 1,000 target nucleotide sequences, at least 10,000 target nucleotide sequences, at least 100,000 target nucleotide sequences, at least 1,000,000 target nucleotide sequences, at least 10,000,000 target nucleotide sequences, at least 100,000,000 target nucleotide sequences, or at least 1,000,000,000 target nucleotide sequences.

In some embodiments, the PIDSs and/or the SIDSs can include oligonucleotides having specific sequences.

In some embodiments, the PIDSs is between 4 and 7 nucleotides, between 8 and 12 nucleotides, between 13 and 16 nucleotides, between 17-20 nucleotides, or greater than 21 nucleotides in length. In some embodiments, the PIDSs is 4, 5, 6, 7, 8, 9, 10, 11, 12, 13, 14, 15, 16, 17, 18, 19, 20, 21, or more nucleotides in length.

In some embodiments, the SIDSs can be between 4 and 7 nucleotides, between 8 and 12 nucleotides, between 13 and 16 nucleotides, between 17-20 nucleotides, or greater than 21 nucleotides in length. In some embodiments, the SIDSs is 4, 5, 6, 7, 8, 9, 10, 11, 12, 13, 14, 15, 16, 17, 18, 19, 20, 21, or more nucleotides in length.

In some embodiments, the PIDSs can include distinct nucleotide sequences chosen from the nucleotide sequences disclosed in Appendix A or Appendix B.

In some embodiments, the SIDS can include distinct nucleotide sequences chosen from the nucleotide sequences disclosed in Appendix A or Appendix B.

In some embodiments, the PIDS and/or the SIDS can include a Raman spectrometry tag, a mass spectrometry tag, or a fluorescent tag (e.g., a quantum dot or a NanoString probe). In some embodiments, the PIDS and/or the SIDS can include a Raman spectrometry tag. In some embodiments, the PIDS and/or the SIDS can include a mass spectrometry tag. In some embodiments, the PIDS and/or the SIDS can include a fluorescent tag.

In some embodiments, quantification of the PIDS and/or the SIDS can be measuring the relative abundance of PIDS and/or SIDS as compared to PIDS and/or SIDS associated with one or more reference TSSs (RTSSs).

In some embodiments, the RTSSs can include OCA2, KLKB, IL4, SETX, PARD3, HIPK3, AMOT, LAMA2, SPAST, and/or PPHLN1, or any combination thereof. In some embodiments, the RTSSs can include OCA2. In some embodiments, the RTSSs can include KLKB. In some embodiments, the RTSSs can include IL4. In some embodiments, the RTSSs can include SETX. In some embodiments, the RTSSs can include PARD3. In some embodiments, the RTSSs can include HIPK3. In some embodiments, the RTSSs can include AMOT. In some embodiments, the RTSSs can include LAMA2. In some embodiments, the RTSSs can include SPAST. In some embodiments, the RTSSs can include PPHLN1.

In some embodiments, at least one of the target nucleotide sequences can be associated with a genetic disorder, cancer, or an infectious disease.

In some embodiments, the genetic disorder can include: spinal muscular atrophy, Duchenne muscular dystrophy, Becker muscular dystrophy, alpha thalassemia, microdeletion and microduplication syndromes associated with neurodevelopmental disorder, autism, atypical hemolytic uraemic syndrome, beta thalassemia, congenital adrenal hyperplasia, thrombophilia, lysosomal storage disorders, Prader-Willi syndrome, Angelmann syndrome, Beckwith-Wiedemann syndrome, Silver-Russell Syndrome, or fragile-X syndrome. In some embodiments, the genetic disorder is spinal muscular atrophy. In some embodiments, the genetic disorder is Duchenne muscular dystrophy. In some embodiments, the genetic disorder is Becker muscular dystrophy. In some embodiments, the genetic disorder is alpha thalassemia. In some embodiments, the genetic disorder is microdeletion and microduplication syndromes associated with neurodevelopmental disorder. In some embodiments, the genetic disorder is autism. In some embodiments, the genetic disorder is atypical hemolytic uraemic syndrome. In some embodiments, the genetic disorder is beta thalassemia. In some embodiments, the genetic disorder is congenital adrenal hyperplasia. In some embodiments, the genetic disorder is thrombophilia. In some embodiments, the genetic disorder is lysosomal storage disorders. In some embodiments, the genetic disorder is Prader-Willi syndrome. In some embodiments, the genetic disorder is Angelmann syndrome. In some embodiments, the genetic disorder is Beckwith-Wiedemann syndrome. In some embodiments, the genetic disorder is Silver-Russell Syndrome. In some embodiments, the genetic disorder is fragile-X syndrome.

In some embodiments, the cancer can include hereditary breast cancer, hereditary ovarian cancer, prostate cancer, renal cancer, cerebellar cancer, colon cancer, or retinoblastoma. In some embodiments, the cancer is hereditary breast cancer. In some embodiments, the cancer is hereditary ovarian cancer. In some embodiments, the cancer is prostate cancer. In some embodiments, the cancer is renal cancer. In some embodiments, the cancer is cerebellar cancer. In some embodiments, the cancer is colon cancer. In some embodiments, the cancer is retinoblastoma

In some embodiments, the infectious disease is caused by chikungunya virus, dengue virus, plasmodium, Zika, cytomegalovirus, Epstein-Barr virus, herpes simplex virus, varicella zoster virus, adenovirus, human immunodeficiency virus, hepatitis B virus, hepatitis C virus, human papillomavirus, Neisseria gonorrhoeae (NG), Chlamydia trachomatis (CT), Trichomonas vaginalis (TV), Mycoplasma sp., influenza virus, S. pneumoniae, K. pneumonia, S. aureus, Salmonella, fungus, Pseudomonas, E. coli, Proteus, Acinetobacter, influenza A virus subtype H1N1, or severe acute respiratory syndrome coronavirus 2 (SARS-CoV-2). In some instances, the infectious disease is caused by influenza A virus subtype H1N1. In some instances, the infectious disease is caused by SARS-CoV-2.

In some embodiments, the PIDS1 and PIDS2 targeting the same target nucleotide sequence can be different from each other or the same.

In some embodiments, the SIDS1 and SIDS2 targeting the same target nucleotide sequence can be different from each other or the same.

In some embodiments, the PIDSs and/or SIDSs can include sequences having an edit distance (Levenshtein) of 2 or more from any other PIDSs and/or SIDSs.

In some embodiments, the TSS can be between 10 and 50 nucleotides, between 15 and 40 nucleotides, or between 20 and 30 nucleotides in length.

In some embodiments, the CA can be between 10 and 60 nucleotides, between 20 and 50 nucleotides, or between 30 and 40 nucleotides in length.

In some embodiments, the target nucleotide sequences can include one or more reference sequences.

In some embodiments, the TSS1 and the TSS2 each can include a nucleic acid sequence that is complementary to at least a portion of the target nucleotide sequence.

In some embodiments, determining the abundance of each of the one or more target nucleotide sequences for each of the one or more samples includes: accessing the quantification results, each of the quantification results being associated with at least one read sequence; classifying the quantification results, using a classifier engine including one or more processing devices, by identifying (i) one of the one or more target nucleotide sequences, and (ii) one of the one or more samples, from each of the corresponding read sequences.

In some embodiments, the at least one read sequence includes a first read sequence usable for identifying one of the one or more target nucleotide sequences, and a second read sequence usable for one of the one or more samples.

In some embodiments, the classifier engine implements a classification process based on a trie search structure.

In some embodiments, the method described herein can include: determining, by the classifier engine, that an edit distance between a particular read sequence and a particular target nucleotide sequence satisfies a threshold condition; and responsive to determining that the edit distance between the particular read sequence and the particular target nucleotide sequence satisfies the threshold condition, identifying the particular read sequence as the particular target nucleotide sequence.

In some embodiments, the threshold condition is determined to be satisfied if the edit distance between the particular read sequence and the particular target nucleotide sequence is less than 3.

In another aspect, the present disclosure can relate to a kit for determining the abundance of each of a plurality of target sequences in each of a plurality of samples, the kit including: (a) a set of TSP1s corresponding to the plurality of target sequences and reference sequences and reference sequences, the set of TSP1s each including, from the 5′ end to the 3′ end, a first common adaptor (CA1), a first PIDS (PIDS1) and a first target-specific sequence (TSS1); (b) a set of TSP2s corresponding to the plurality of target sequences and reference sequences, the set of TSP1s each including, from the 5′ end to the 3′ end, a second target-specific sequence (TSS2), a second PIDS (PIDS2) and a second common adaptor (CA2); (c) a set of first PCR primers including, from the 5′ end to the 3′ end, a first tethering adaptor (TA1), a first SIDS (SIDS1), and a sequence corresponding to the CA1; (d) a set of second PCR primers including, from the 5′ end to the 3′ end, a second tethering adaptor (TA2), a second SIDS (SIDS2), and a sequence corresponding to the CA2; and (e) optionally, a ligase and/or a polymerase.

In another aspect, the present disclosure can relate to a kit for determining the abundance of each of a plurality of target sequences having specific sequences in each of a plurality of samples, the kit including: (a) a set of first primers corresponding to the plurality of target sequences and reference sequences, the set of first primers each including, from the 5′ end to the 3′ end, a first common adaptor (CA1), a first PIDS (PIDS1), and a first TSS (TSS1), thereby generating first intermediary PCR products (IPP1); (b) a set of second primers corresponding to the plurality of target sequences and reference sequences, the set of second primers each including, from the 5′ end to the 3′ end, a second common adaptor (CA2), a second PIDS (PIDS2), and a second TSS (TSS2), thereby generating second intermediary PCR products (IPP2); (c) a set of third primers corresponding to the sequences of the CA1, the set of second primers each including, from the 5′ end to the 3′ end, a first Tethering Adapter (TA1), a first SIDS (SIDS1), and a sequence corresponding to CA1; (d) a set of fourth primers corresponding to the sequences of the CA2, the set of second primers each including, from the 5′ end to the 3′ end a second Tethering adapter (TA2), a second SIDS (SIDS2), and a sequence corresponding to CA2; and (e) optionally, a polymerase.

In another aspect, the present disclosure can relate to a method of diagnosing one or more conditions in one or more subjects by detecting the presence or absence of one or more nucleic acid alteration in the plurality of subjects, the method including: (a) obtaining a plurality of samples from the plurality of subjects; (b) performing a method of determining the abundance of target nucleotide sequences in samples described herein to determine the abundance of each of the plurality of target genes in each of the plurality of samples; and (c) diagnosing the one or more conditions that are each associated with the abundance of one or more of the plurality of target genes for each of the plurality of samples.

In some embodiments, the method of diagnosing one or more conditions in one or more subjects can further include treating the subjects for the condition diagnosed.

As used herein, the term “abundance” with respect to a target nucleotide sequence can mean presence or absence of the target nucleotide sequence, copy number of the target nucleotide sequence, or quantity (absolute or relative) of the target nucleotide sequence.

As used herein, the terms “corresponding to,” “correspond to” or “corresponds to” can mean, when recited with respect to between two nucleotide sequences, having identical nucleotide sequences, having complementary nucleotide sequences, or having reverse-complementary sequences between the two nucleotide sequences.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### BRIEF DESCRIPTION OF THE DRAWINGS

FIG. 1 is a schematic overview of a method for detecting multiple target sequences (Target Sequences A-X) from each of multiple samples (Samples 1-N) by a single analysis using the method described in this disclosure.

FIGS. 2A-2D show target sequences that can be used to generate the nucleotide constructs.

FIGS. 3A-3D show binding of first target-specific probes (TSP1s) and second target-specific probes (TSP2s) to corresponding target sequences from FIGS. 2A-2D, respectively.

FIGS. 4A-4D show ligation of TSP1s and TSP2s that are bound to their corresponding target sequences and adjacent to each other.

FIGS. 5A-5D show ligation products (LPs) containing PIDS1, PIDS2, first common adapters (CA1) and second common adapters (CA2), formed by ligation of TSP1s and TSP2s.

FIGS. 6A-6D show binding of PCR primers containing first tethering adaptors (TA1s), SIDS's, CA1s to the LPs from FIGS. 5A-5D, respectively.

FIGS. 7A-7D show PCR amplification of the LPs using the PCR primers from FIGS. 6A-6D, respectively.

FIGS. 8A-8D show binding of PCR primers containing second tethering adaptors (TA2s), SIDS2s, CA2s to the amplified products from FIGS. 7A-7D, respectively, and amplification of the PCR products from FIGS. 7A-7D, respectively.

FIGS. 9A-9D show nucleotide constructs containing PIDSs and SIDSs produced by the PCR amplification step of FIGS. 8A-8D, respectively.

FIGS. 10A-10D show target sequences that can be used to generate the nucleotide constructs by extension-ligation approach.

FIGS. 11A-11D show binding of TSP1s and TSP2s to corresponding target sequences from FIGS. 10A-10D, respectively.

FIGS. 12A-12D show extension and ligation of TSP1s and TSP2s that are bound to their corresponding target sequences.

FIGS. 13A-13D show LPs containing PIDS1s, PIDS2s, CA1s and CA2s, formed by extension of TSP1s at the 3′ ends and ligation of extended TSP1s and TSP2s.

FIGS. 14A-14D show binding of PCR primers containing TAs, SIDS1s, CA1s to the LPs from FIGS. 13A-13D, respectively.

FIGS. 15A-15D show amplification of the LPs using the PCR primers from FIGS. 6A-6D, respectively, subsequent binding of second set of PCR primers containing TAs, SIDS2s, CA2s to the amplified products, and second round of PCR amplification to produce the nucleotide constructs containing PIDS and SIDS.

FIGS. 16A-D show the nucleotide constructs containing PIDS and SIDS produced by the two consecutive PCR amplification steps of FIGS. 15A-15D.

FIGS. 17A-E are schematics showing preparation of nucleotide constructs containing PIDS and SIDS by PCR using the method described in this disclosure.

FIG. 17A shows binding of a first primer containing PIDS1 to a target sequence and subsequent amplification to generate a first intermediary PCR product (IPP1).

FIG. 17B shows binding of a second primer containing PIDS2 to the IPP1 and subsequent amplification to generate a second intermediary PCR product (IPP2).

FIG. 17C shows the IPP3 generated in FIG. 17B.

FIG. 17D shows binding of a third primer containing SIDS1 to the IPP2 and subsequent amplification to generate a third intermediary PCR product (IPP3).

FIG. 17E shows binding of a third primer containing SIDS2 to the IPP3 from FIG. 17D and subsequent amplification to generate the nucleotide construct containing PIDS and SIDS.

FIG. 18 shows a block diagram of an example system usable for implementing a portion of the technology described herein.

FIG. 19 shows a flowchart of an example process for determining the abundance of each of the one or more target nucleotide sequences for each of the one or more samples.

FIG. 20 shows a block diagram of an example computer system that can be used to perform operations described herein.

### DETAILED DESCRIPTION

Advances in molecular diagnostic technologies such as NGS have enabled high-throughput detection of nucleotide sequences (e.g., genomic DNA sequences), including detection of low-depth genomic variants in testing samples. However, there still remain challenges with NGS, e.g., with respect to detection and analysis of certain genetic variations (e.g., CNVs, DelDup, and LGRs), unequal coverage of targets, sequence-dependent biases, handling of large-sized data that is generated by NGS analysis, and limits to its scalability (e.g., when screening for large number of genes in multiple subjects).

Accordingly, the present disclosure provides methods that allow highly multiplexed analysis of a large number of genetic sequences (e.g., CNVs, DelDup, LGRs, and those from infectious agents) in a large number of samples (e.g., from multiple subjects or multiple samples from the same subject) in a single sequence analysis (e.g., NGS). The methods are performed, in some instances, by generating nested multi-indexed nucleotide constructs for sequence analysis as proxies for the target sequences.

In some instances, the present disclosure provides multiplexed analysis using at least one of the target nucleotide sequences that is associated with a genetic disorder. In some instances, the present disclosure provides multiplexed analysis using at least one of the target nucleotide sequences that is associated with a cancer. In some instances, the present disclosure provides multiplexed analysis using at least one of the target nucleotide sequences that is associated with a genetic disorder. infectious disease.

In some embodiments, the genetic disorder can include spinal muscular atrophy, Duchenne muscular dystrophy, Becker muscular dystrophy, alpha thalassemia, microdeletion and microduplication syndromes associated with neurodevelopmental disorder, autism, atypical hemolytic uraemic syndrome, beta thalassemia, congenital adrenal hyperplasia, thrombophilia, lysosomal storage disorders, Prader-Willi syndrome, Angelmann syndrome, Beckwith-Wiedemann syndrome, Silver-Russell Syndrome, or fragile-X syndrome. In some embodiments, the genetic disorder is spinal muscular atrophy. In some embodiments, the genetic disorder is Duchenne muscular dystrophy. In some embodiments, the genetic disorder is Becker muscular dystrophy. In some embodiments, the genetic disorder is alpha thalassemia. In some embodiments, the genetic disorder is microdeletion and microduplication syndromes associated with neurodevelopmental disorder. In some embodiments, the genetic disorder is autism. In some embodiments, the genetic disorder is atypical hemolytic uraemic syndrome. In some embodiments, the genetic disorder is beta thalassemia. In some embodiments, the genetic disorder is congenital adrenal hyperplasia. In some embodiments, the genetic disorder is thrombophilia. In some embodiments, the genetic disorder is lysosomal storage disorders. In some embodiments, the genetic disorder is Prader-Willi syndrome. In some embodiments, the genetic disorder is Angelmann syndrome. In some embodiments, the genetic disorder is Beckwith-Wiedemann syndrome. In some embodiments, the genetic disorder is Silver-Russell Syndrome. In some embodiments, the genetic disorder is fragile-X syndrome.

In some embodiments, the cancer can include hereditary breast cancer, hereditary ovarian cancer, prostate cancer, renal cancer, cerebellar cancer, colon cancer, or retinoblastoma. In some embodiments, the cancer is hereditary breast cancer. In some embodiments, the cancer is hereditary ovarian cancer. In some embodiments, the cancer is prostate cancer. In some embodiments, the cancer is renal cancer. In some embodiments, the cancer is cerebellar cancer. In some embodiments, the cancer is colon cancer. In some embodiments, the cancer is retinoblastoma

In some embodiments, the infectious disease is caused by chikungunya virus, dengue virus, plasmodium, Zika, cytomegalovirus, Epstein-Barr virus, herpes simplex virus, varicella zoster virus, adenovirus, human immunodeficiency virus, hepatitis B virus, hepatitis C virus, human papillomavirus, Neisseria gonorrhoeae (NG), Chlamydia trachomatis (CT), Trichomonas vaginalis (TV), Mycoplasma sp., influenza virus, S. pneumoniae, K. pneumonia, S. aureus, Salmonella, fungus, Pseudomonas, E. coli, Proteus, Acinetobacter, influenza A virus subtype H1N1, or severe acute respiratory syndrome coronavirus 2 (SARS-CoV-2). In some instances, the infectious disease is caused by influenza A virus subtype H1N1. In some instances, the infectious disease is caused by SARS-CoV-2.

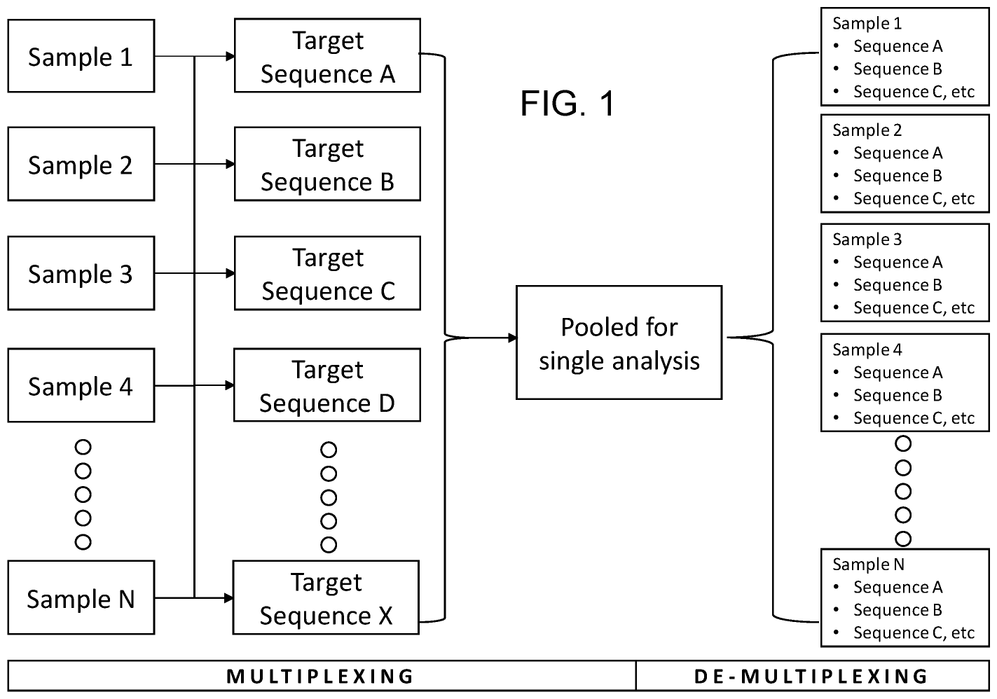

As shown in FIG. 1, the present disclosure provides highly multiplexed methods for detecting multiple target sequences (Target Sequence A-X) from multiple samples (Sample 1-N) using a single analysis step. The highly multiplexed data generated from the single analysis step can be “demultiplexed” to provide information on the abundance (e.g., presence/absence, or relative abundance) of each of the multiple target sequences in each of the multiple subjects (see right side panels in FIG. 1, showing abundance of Sequence A, Sequence, B, Sequence C, etc in each of Samples 1-N).

In some instances, methods described herein can be used to screen a large number of subjects for multiple classes of genetic or epigenetic information (e.g., presence or absence of genetic aberrations, chromosomal abnormalities, copy number variations, and/or methylation status) in a single gene sequencing analysis (e.g., using a next-generation sequencing platform). In some instances, methods described herein can be used to diagnose infections by determining the presence or absence of specific nucleic acid sequences (i.e., target sequences) associated with infectious agents (e.g., viruses, bacteria, or fungi). In other instances, methods described herein can be used to determine the pharmacogenetic profile (e.g., suitability of a certain drug to treat certain condition in a subject) for subjects based on genotype analysis of subjects.

In some instances, the present disclosure is based on ultra-short reads NGS coupled with a dual indexing strategy which enables highly multiplexed analysis of multiple targets in multiple samples (e.g. ˜6000 samples with 18 targets per sample can be processed in a single run of a sequencer with the capacity similar to an Illumina NextSeq in HiOutput mode).

### Nucleotide Constructs Having Nested Multi-Indexed Identifiers

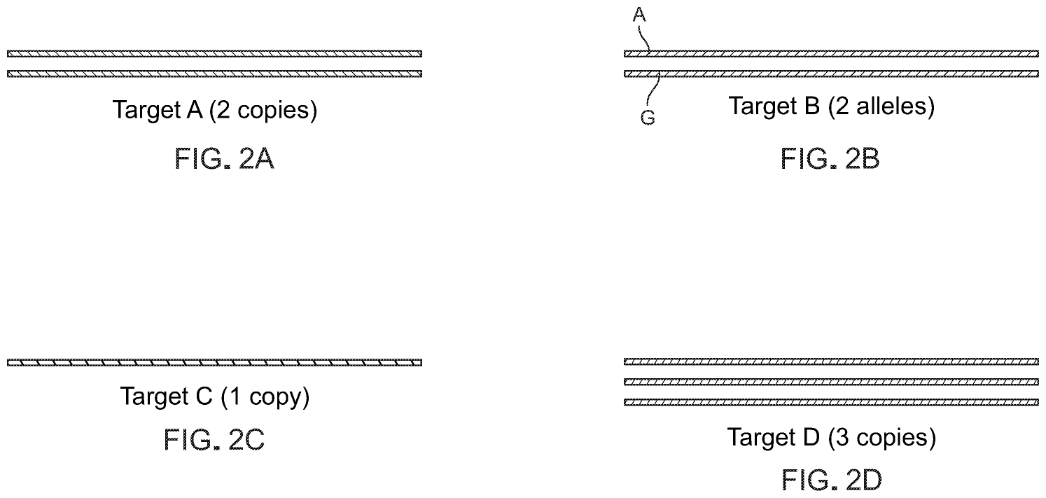

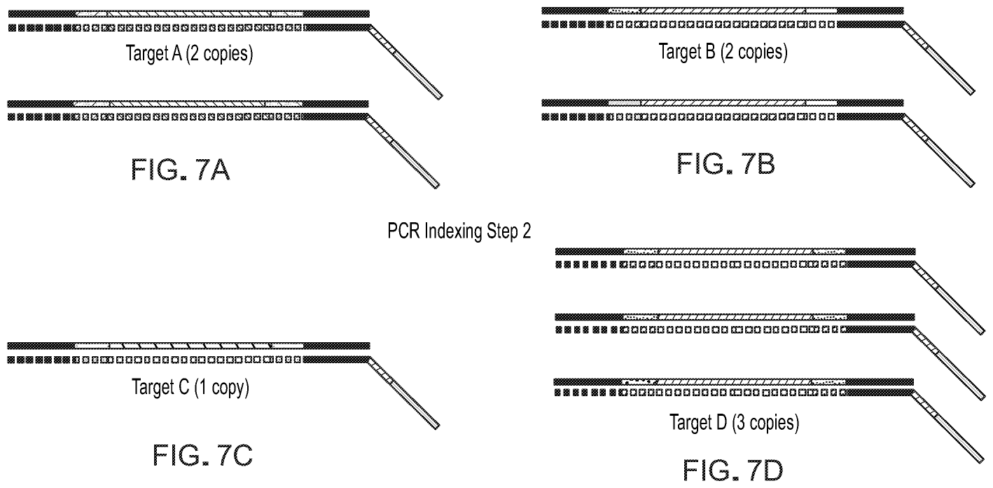

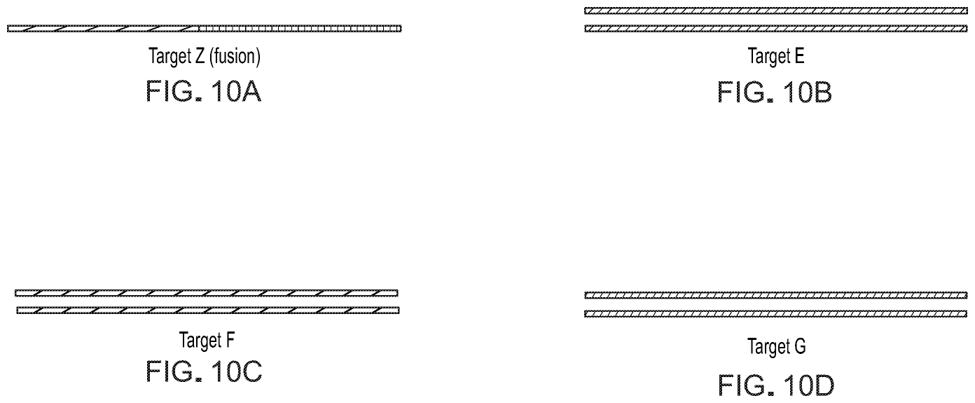

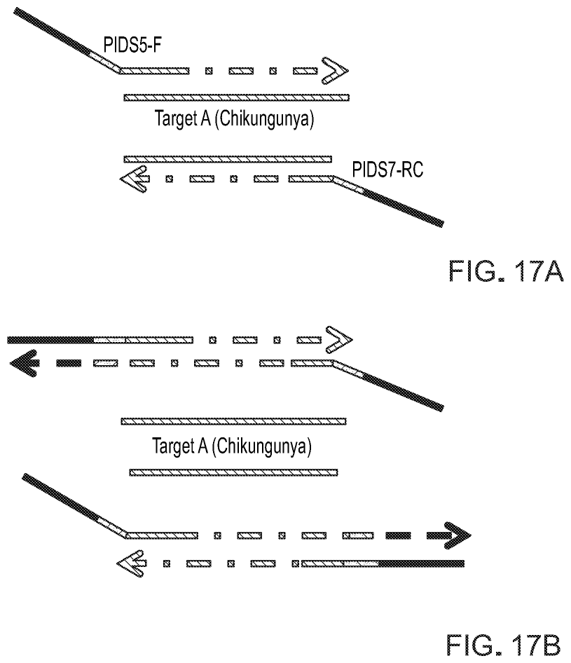

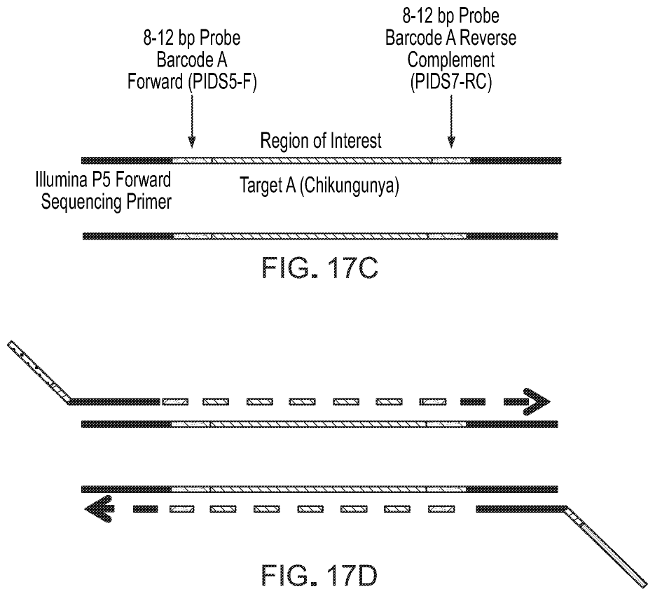

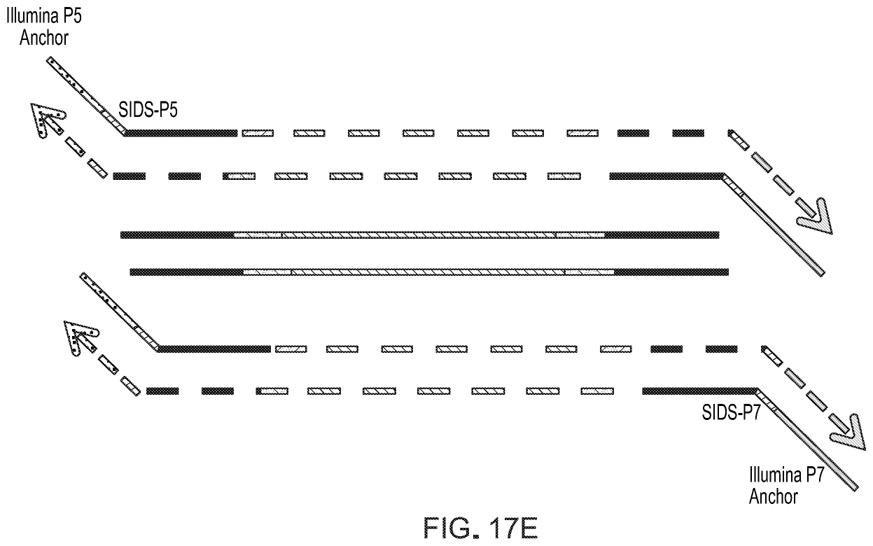

Certain methods described herein involve generating nucleotide constructs that include: (1) nucleic acid sequences that correspond to (e.g., are matching or complementary to) the target nucleotides and (2) multi-indexed identifiers (e.g., PIDS and/or SIDS). Such nucleotide constructs can be generated by a number of different methods, including ligation method (see FIG. 2A-9D), extension-ligation method (see FIG. 10A-16D), or PCR method (see FIG. 17A-E).

### Ligation Method for Generation of Nucleotide Sequences Having Nested Multi-Indexed Identifiers

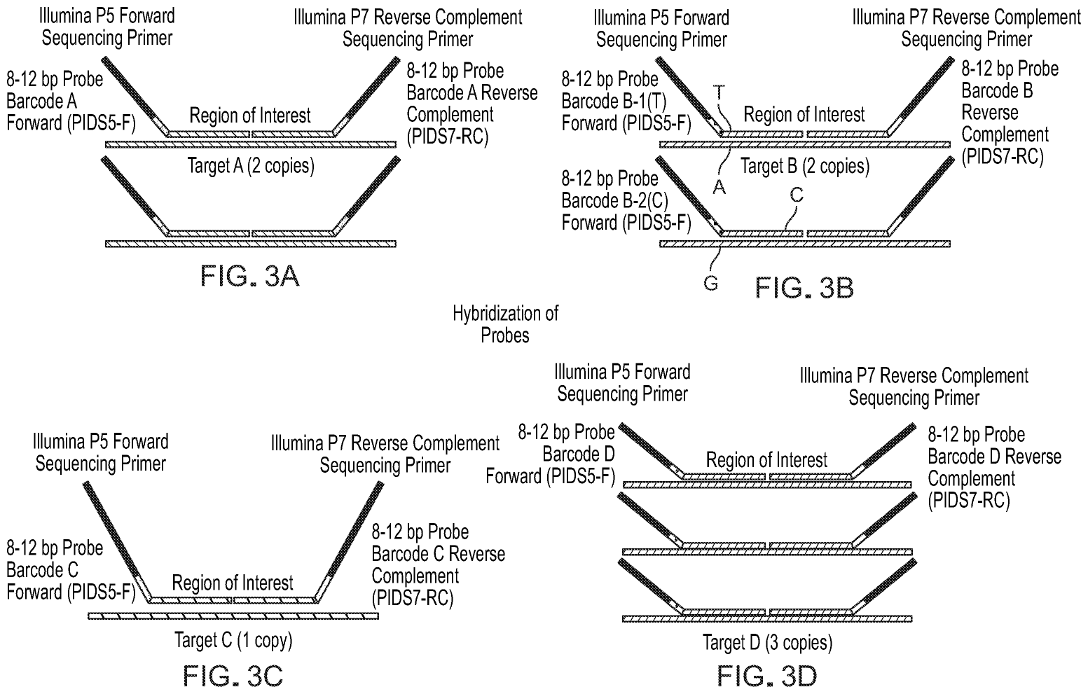

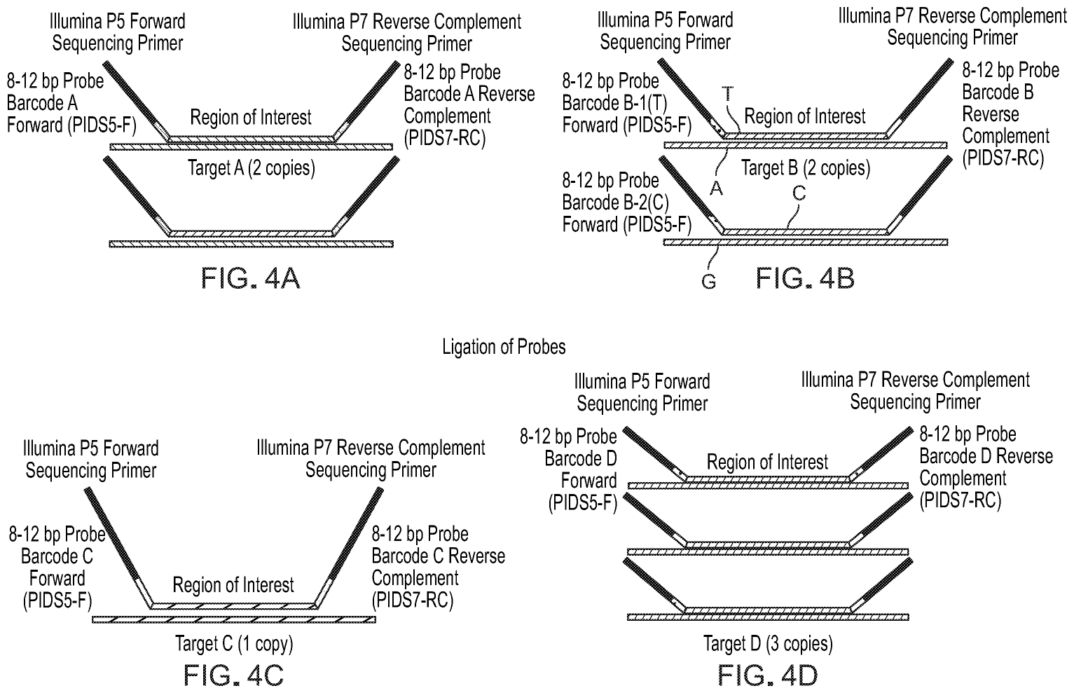

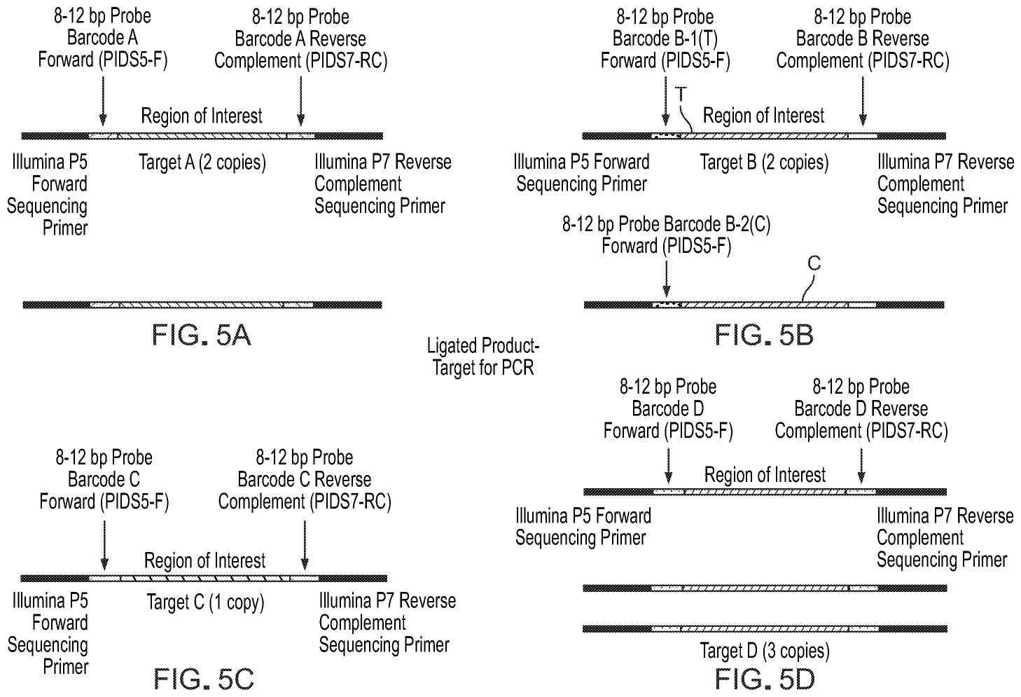

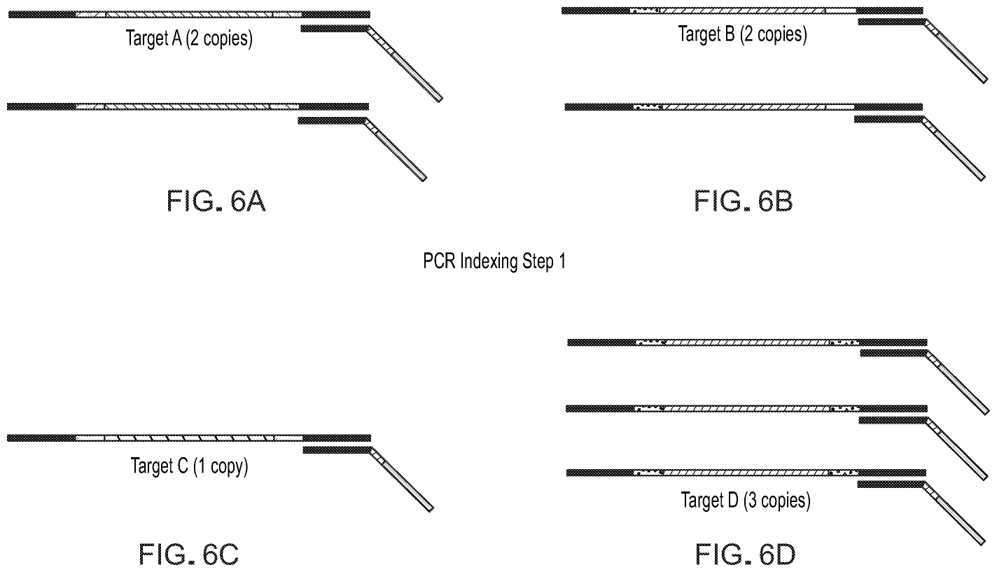

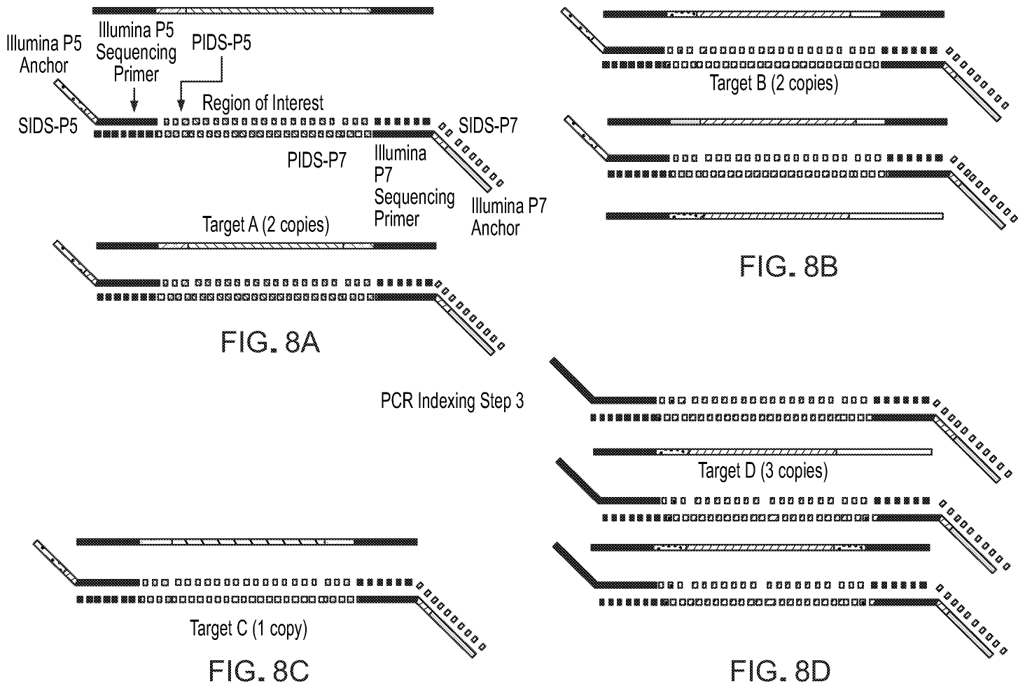

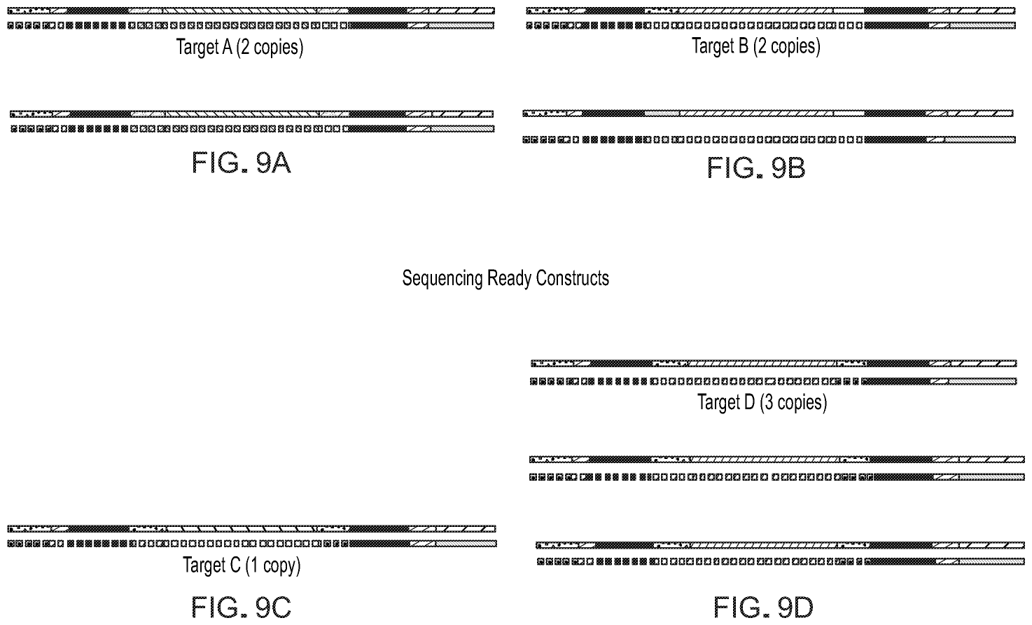

Nucleotide constructs containing PIDS and SIDS can be generated from target sequences by ligation methods as shown in FIGS. 2A-9D. FIGS. 2A-9D are schematics showing preparation of nucleotide constructs containing probe identification sequences (PIDSs) and sample identification sequences (SIDSs) by ligation method using the method described in this disclosure. As shown there, nucleotide constructs can be generated to detect various different types of target sequences (e.g., having different genetic abnormalities such as CNVs or point mutations). FIGS. 2A, 2C, and 2D show target sequences having different copy numbers (2 copies, 1 copy, and 3 copies, respectively). FIG. 2B shows a target sequence having a mismatch (A-G mismatch). Next, a pair of target sequence-specific probes (TSP1 and TSP2) containing CAs (common adapters), PIDSs and target-specific sequences (TSSs) can be hybridized to each of the target sequences (see FIGS. 3A-D) and a ligase is added, to ligate those TSP1s and TSP2s that are adjacent to each other (without gaps) (see FIGS. 4A-D) to generate LPs (ligated products) (see FIGS. 5A-D). Next, the LPs are amplified (e.g., sequentially) using a first PCR primer (see FIGS. 6A-D and 7A-D) and a second PCR primer (see FIGS. 8A-D), each comprising TAs (tethering adapters), SIDSs, and sequences corresponding to CAs, to generate the nucleic acid constructs (see FIGS. 9A-D).

### Extension-Ligation Method for Generation of Nucleotide Sequences Having Nested Multi-Indexed Identifiers

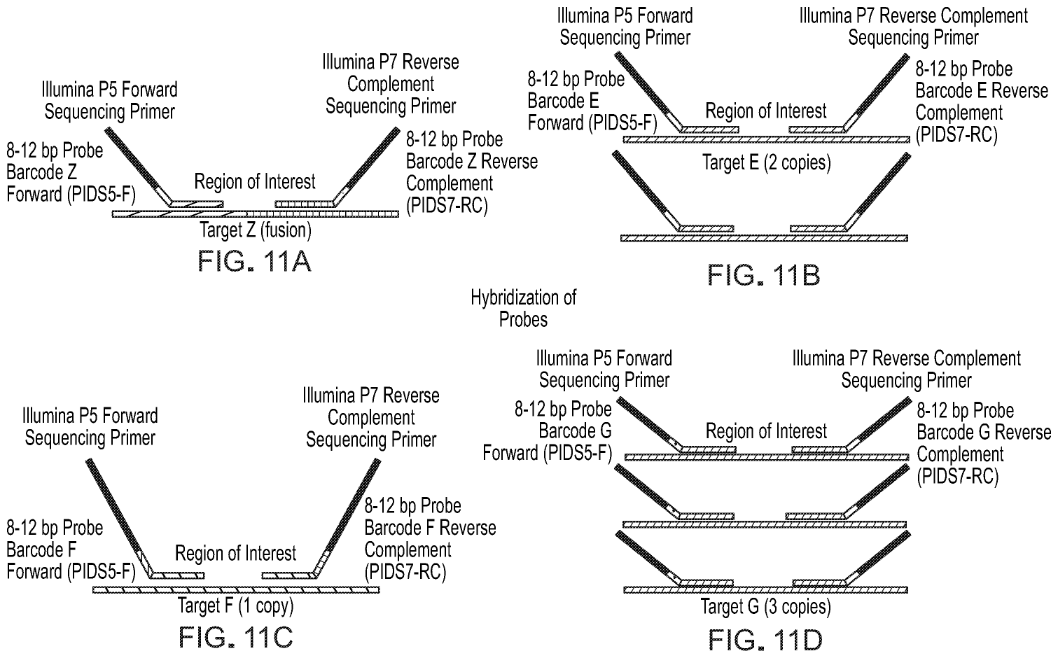

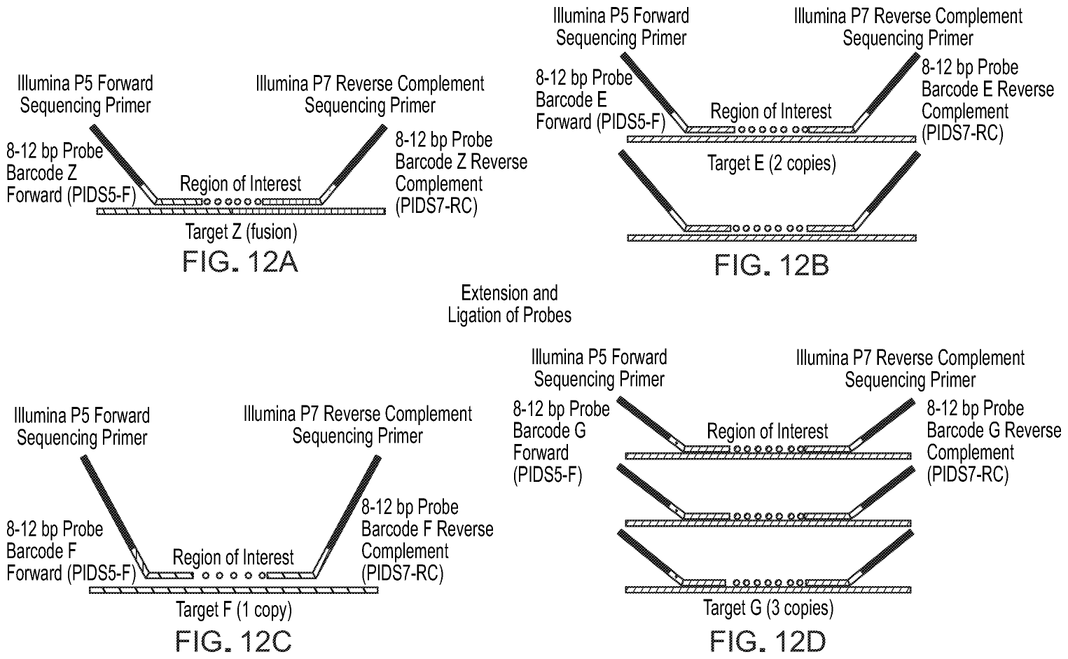

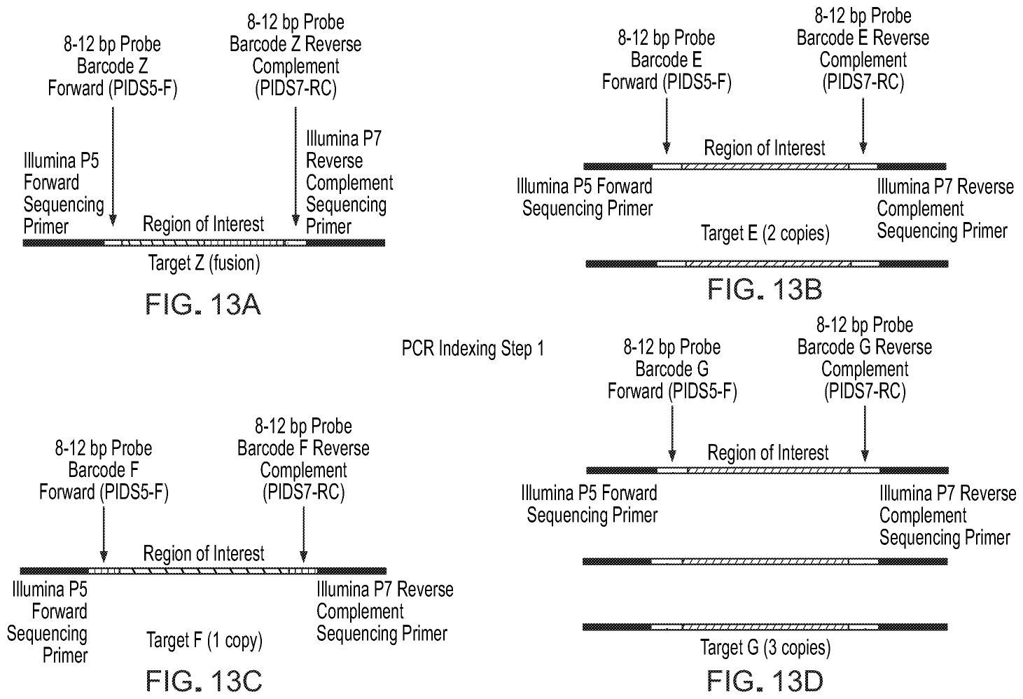

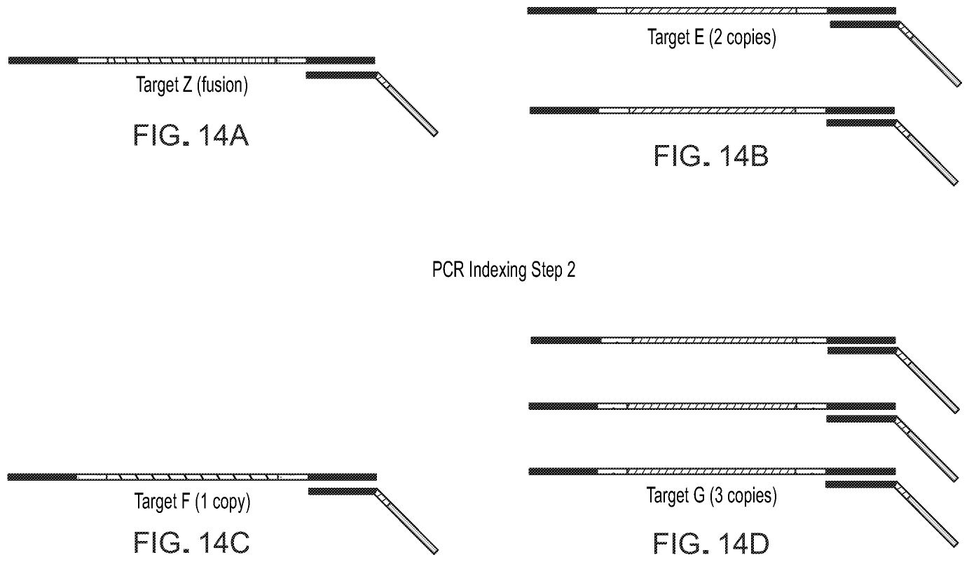

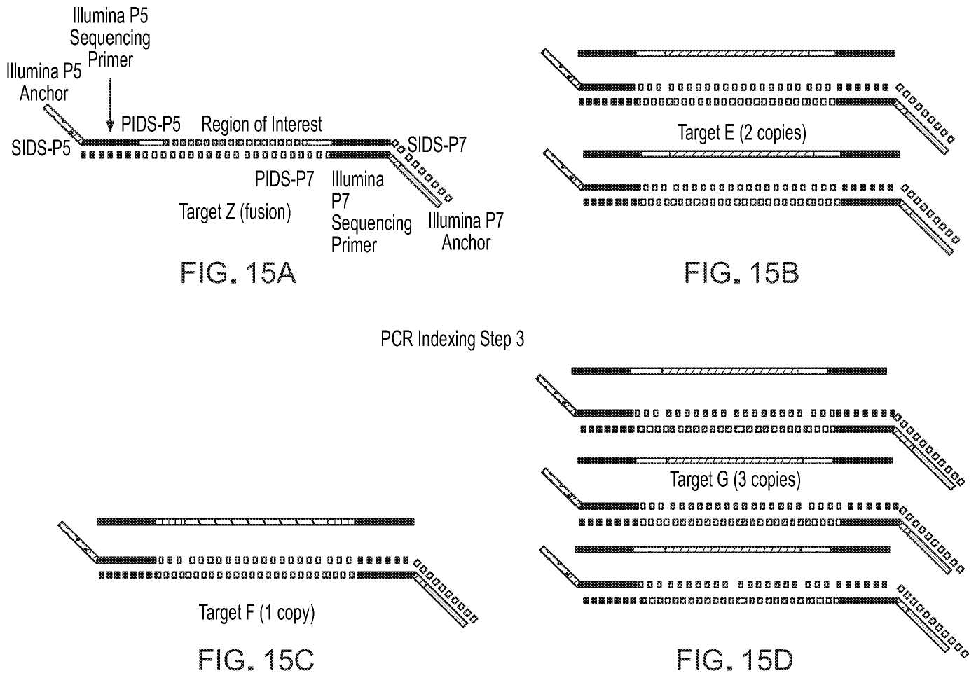

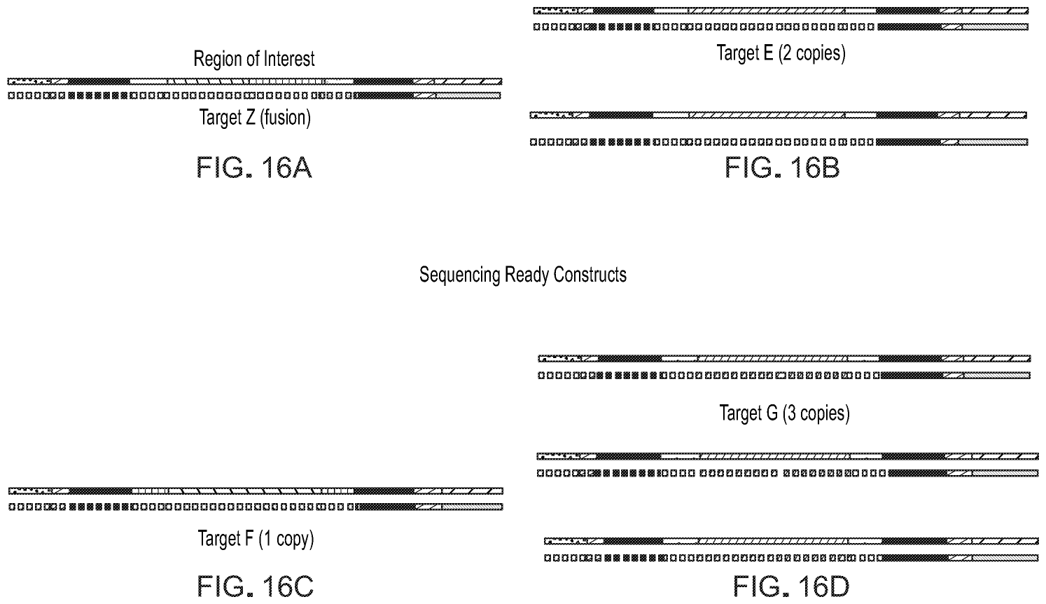

Nucleotide constructs including PIDS and SIDS can be generated from target sequences by an extension-ligation method such as that shown in FIGS. 10A-16D. FIGS. 10A-16D are schematics showing preparation of nucleotide constructs containing PIDS and SIDS by extension-ligation method using the method described in this disclosure. As shown there, nucleotide constructs can be generated to detect various different types of target sequences (e.g., having different genetic abnormalities such as gene fusions (FIG. 10A) or target sequences having different sequences (FIGS. 10B-D)). Next, a pair of target sequence-specific probes (TSP1 and TSP2) containing CAs, PIDSs and TSSs are hybridized to each of the target sequences (see FIGS. 11A-D). Unlike in the ligation method, the two probes do not need to be adjacent to each other, and a gap can exist between the two probes. Next, a polymerase and appropriate other reagents (e.g., nucleotides) are added to extend the 3′ end of TSP1 so that any gap between TSP1 and TSP2 are closed, and the two probes are adjacent to each other (see FIGS. 12A-D), and a ligase is added to ligate those TSP1 and TSP2 that are adjacent to each other, thereby generating LPs (see FIGS. 13A-D). Next the LPs are amplified (e.g., sequentially) using a first PCR primer (see FIGS. 14A-D) and a second PCR primer (see FIGS. 15A-D), each comprising TAs, SIDSs, and sequences corresponding to CAs, to generate the nucleic acid constructs (see FIGS. 16A-D).

PCR method for Generation of Nucleotide Sequences Having Nested Multi-Indexed Identifiers

Nucleotide constructs containing PIDS and SIDS can be generated from target sequences by PCR method as shown in FIGS. 17A-17E. As shown there, a target sequence can be amplified using a first primer containing CA1, a PIDS1, and a TSS1 to generate a first intermediary PCR product (IPP1). The IPP1 contains PIDS1 and CA1. Next, a second primer containing CA2, PIDS2, and TSS2 can be used to generate a second intermediary PCR product (IPP2), which contains CA1, CA2, PIDS1, and PIDS2 (see FIG. 17C). Next, a third primer containing a TA1, a SIDS1, and a sequence corresponding to CAL can be used to generate a third intermediary PCR product (IPP3), which includes TA1 and SIDS1, in addition to the other components contained in IPP2. Lastly, a fourth primer containing a TA2, a SIDS2, and a sequence corresponding to CA2 can be used to generate the nucleotide construct, which contains PIDSs, SIDSs, CAs, and TAs (see FIG. 17E).

### Pooling of the Nucleotide Constructs for Analysis

Once the nucleotide constructs are generated from the target nucleic acid sequences from the samples, the nucleotide constructs from the different samples can be pooled or combined for a single analysis. This single analysis of nucleotide constructs from multiple samples enables higher throughput analysis of target sequences in multiple samples (e.g., screening for multiple genetic aberrations in large number of patients, or screening for multiple genetic aberrations in different samples obtained from the same patient) which can provide logistical and economic benefits, improve access to diagnostics services to patients, and/or provide healthcare providers with improved information relevant to provide appropriate healthcare services to subjects. One of the ways the present invention enables pooling of the samples for single analysis is the use of multi-indexed identifiers that can be incorporated into nucleotide constructs that derive from samples (e.g., blood, urine, spinal fluid). The nucleotide constructs derived from target sequences in samples can be used to detect the abundance of the identifiers (e.g., PIDSs and/or SIDSs) that are present in the nucleotide constructs, and this information can be used to quantify both the abundance (e.g., presence or absence, or relative quantity) and source (e.g., the sample the target sequence was obtained from) of the target sequences that are associated with each of the nucleotide constructs.

### Identifiers for Use as Multi-Indexed Identifiers in Nucleotide Constructs

Identifiers (e.g., PIDSs and SIDSs) described herein can be oligonucleotides, fluorescent tags, Raman spectrometry tags, or mass spectrometry tags. The identifiers can be other forms of molecules that can provide unique identifying information, such that detection or quantification of the identifiers can be used as a proxy to determine the identity of corresponding target nucleic acid sequences, the abundance (e.g., presence or absence, or relative quantity) of the specific target nucleic acid sequence and/or identify the specific sample from which the specific target nucleic acid sequence is obtained.

In order for a set of identifiers to provide information on the identity and abundance of corresponding target nucleic acid sequence and/or the sample source, the set of identifiers must be distinguishable from each other. For example, if the identifier is in the form of oligonucleotides, the sequence of the oligonucleotide identifiers can be used (e.g., by NGS analysis) to distinguish from one another.

One advantage of using this approach to determining the abundance of a target nucleic acid sequence is the relative short length of the identifier oligonucleotide sequences (e.g., 4-7 nt, 8-12 nt, 13-16 nt, 17-20 nt, or greater than 21 nt) that needs to be sequenced compared to the length of target nucleic acid sequence that is typically sequenced (e.g., read length when using NGS analysis).

Another advantage of this approach, in certain examples provided herein is the ability to incorporate common adapters (CA1 and/or CA2) between two different identifiers (e.g., between PIDS and SIDS), which allows use of common sequencing primers that can potentially be used to analyze large number of target nucleic acid sequences in large sample size.

### Scalability of the Present Method

One of the features of the methods described herein is the ability to screen, in a single analysis (e.g., using NGS), large number of target sequences in large number of samples. The methods can be scaled to accommodate an extremely large number of target sequences (e.g., at least 100 target nucleotide sequences, at least 1,000 target nucleotide sequences, at least 10,000 target nucleotide sequences, at least 100,000 target nucleotide sequences, at least 1,000,000 target nucleotide sequences, at least 10,000,000 target nucleotide sequences, at least 100,000,000 target nucleotide sequences, or at least 1,000,000,000 target nucleotide sequences) in an extremely large number of samples (at least 100 samples, at least 1,000 samples, at least 10,000 samples, at least 100,000 samples, at least 1,000,000 samples, at least 10,000,000 samples, at least 100,000,000 samples, or at least 1,000,000,000 samples). This scalability is in part based on the ability to generate an extremely large number of distinct identifiers (e.g., 10 nt long oligonucleotide can theoretically have 1,048,576 different sequences; 20 nt long oligonucleotide can theoretically have over 10¹² different sequences), and in part, the ability for the analysis platform (e.g., NGS) that can perform extremely large distinct sequencing reactions. As technology in the analysis platform improves, the scalability of the present invention can also improve.

### Demultiplexing Sample Specific Read Sequences

In some implementations, the output of the sequencer is processed by a classification engine 1815 executing on one or more computing devices to demultiplex the reads. In some implementations, the classifier engine 1815 can be configured to execute a software package such as the Illumina bcl2fastq software.

Kits for Use in Accordance with the Present Specification

The present disclosure also provides for kits that can be used to carry out the methods described herein. The kits can contain some or all of the key components necessary for carrying out the various steps of the methods described herein.

In one instance, a kit can comprise sets of TSP1s, TSP2s, each containing appropriate CAs, PIDSs, and TSSs that corresponds to a target sequence and reference sequence(s); a first set of first and second PCR primers, each containing appropriate TAs, SIDSs, and sequences corresponding to CAs of the TSP1s and TSP2s. The kit can optionally also provide a ligase, a polymerase, and other reagents useful for ligation and/or nucleic acid extension and amplification. Such a kit can be used for ligation methods or extension-ligation methods described herein, for generating nucleotide constructs useful in detecting and quantifying target sequences in samples.

In another instance, a kit can comprise sets of first primers, second primers, third primers, and fourth primers described herein for the PCR method for generation of nucleic acid constructs. The first and second primers each can contain a CA, a PIDS, and a TSS corresponding to a target sequence. The third and fourth primers each can contain a TA, a SIDS, and a sequence corresponding to a CA of the first or second primer. Optionally, the kit can also contain other reagents, such as polymerases and nucleotides that are used in PCR amplification. Such a kit can be used for the PCR method described herein to generate nucleotide constructs for use in detection and quantification of target sequences in samples.

### Processes for Implementation of the Present Specification

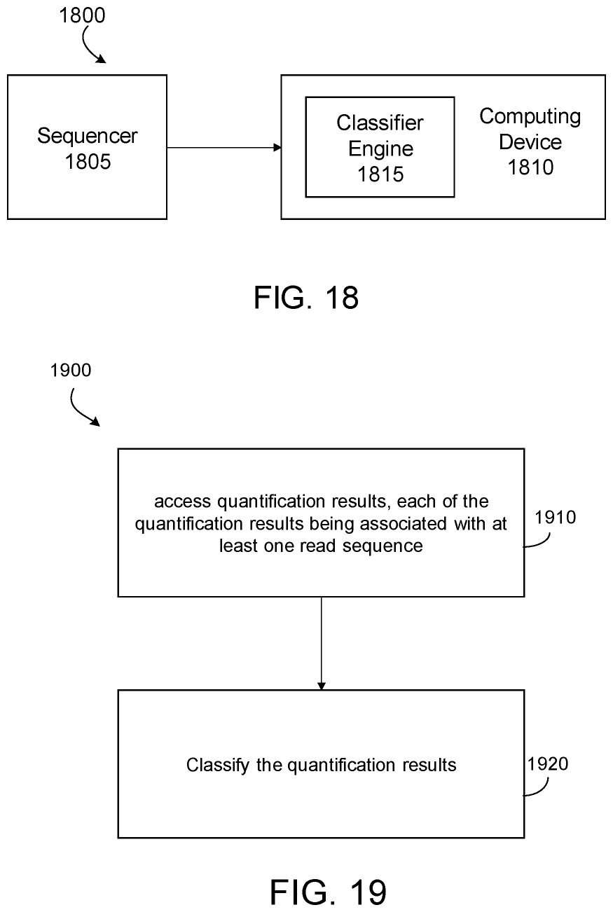

FIG. 19 is a flowchart of an example process 1900 for determining the abundance of each of the one or more target nucleotide sequences for each of the one or more samples. In some implementations, at least a portion of the operations of the process 1900 is executed by the classifier engine 1815 described above with reference to FIG. 18. Operations of the process 1900 includes accessing the quantification results generated by a sequencer (1910), wherein each of the quantification results is associated with at least one read sequence. In some implementations, the sequencer is substantially similar to the sequencer 1805 described above with reference to FIG. 18. In some implementations, the at least one read sequence can include a first read sequence usable for identifying the one of the one or more target nucleotide sequences. In some implementations, he at least one read sequence can include a second read sequence usable for one of the one or more samples.

Operations of the process 1900 also includes classifying the quantification results (1920). This can be done, for example, by identifying (i) one of the one or more target nucleotide sequences, and (ii) one of the one or more samples, from each of the corresponding read sequences. In some implementations, the process 1900 further includes determining, by the classifier engine, that an edit distance between a particular read sequence and a particular target nucleotide sequence satisfies a threshold condition, and in response, identifying the particular read sequence as the particular target nucleotide sequence. The threshold condition can be determined to be satisfied if the edit distance between the particular read sequence and the particular target nucleotide sequence is less than a particular value such as 3, 4, or 5. In some implementations, the classifier engine implements a classification process based on a trie search structure such as the ones described above.

### Systems for Implementation of the Present Specification

FIG. 18 shows a block diagram of an example system 1800 usable for implementing a portion of the technology described herein. Specifically, the system 1800 includes a sequencer 1805 that provides input to a computing device 1810. In some implementations, the computing device 1810 is a special purpose device that includes a classifier engine 1815 for implementing demultiplexing operations as described herein. The term “engine” is used broadly to refer to a software-based system, subsystem, or process that is programmed to perform one or more specific functions. Generally, an engine will be implemented as one or more software modules or components, installed on one or more computers in one or more locations. In some cases, one or more computers will be dedicated to a particular engine; in other cases, multiple engines can be installed and running on the same computer or computers. In some implementations, the classifier engine 1815 may execute on one or more servers that are remote with respect to the sequencer 1805. In such cases, the sequencer can be communicably connected to the classifier engine over one or more computer networks including, for example, a local area network (LAN), a wide area network (WAN), and/or the Internet.

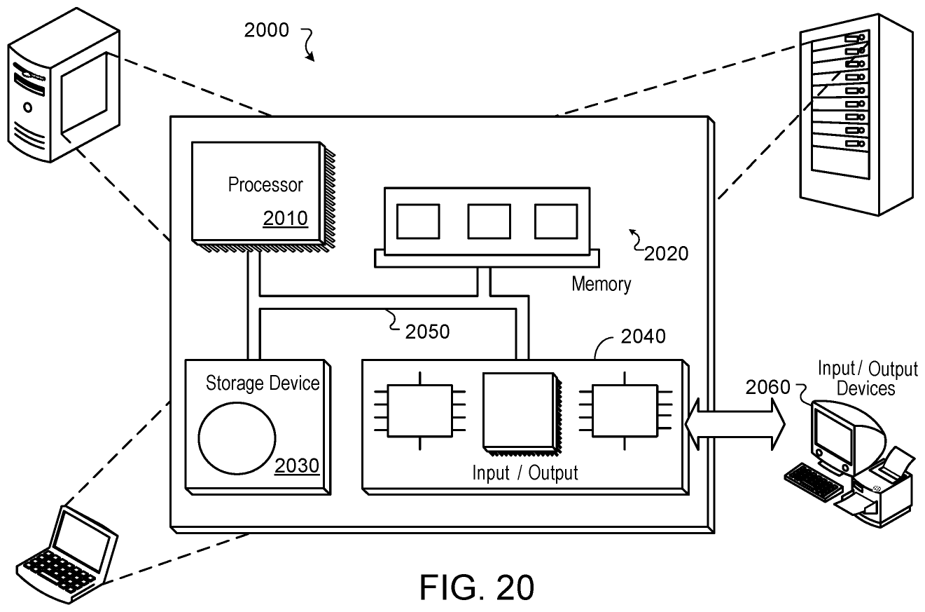

FIG. 20 is block diagram of an example computer system 2000 that can be used to perform operations described above. The system 2000 includes a processor 2010, a memory 2020, a storage device 2030, and an input/output device 2040. Each of the components 2010, 2020, 2030, and 2040 can be interconnected, for example, using a system bus 2050. The processor 2010 is capable of processing instructions for execution within the system 2000. In one implementation, the processor 2010 is a single-threaded processor. In another implementation, the processor 2010 is a multi-threaded processor. The processor 2010 is capable of processing instructions stored in the memory 2020 or on the storage device 2030.

The memory 2020 stores information within the system 2000. In one implementation, the memory 2020 is a computer-readable medium. In one implementation, the memory 2020 is a volatile memory unit. In another implementation, the memory 2020 is a non-volatile memory unit.

The storage device 2030 is capable of providing mass storage for the system 2000. In one implementation, the storage device 2030 is a computer-readable medium. In various different implementations, the storage device 2030 can include, for example, a hard disk device, an optical disk device, a storage device that is shared over a network by multiple computing devices (e.g., a cloud storage device), or some other large capacity storage device.

The input/output device 2040 provides input/output operations for the system 900. In one implementation, the input/output device 2040 can include one or more network interface devices, e.g., an Ethernet card, a serial communication device, e.g., and RS-232 port, and/or a wireless interface device, e.g., and 802.11 card. In another implementation, the input/output device can include driver devices configured to receive input data and send output data to other input/output devices, e.g., keyboard, printer and display devices 960. Other implementations, however, can also be used, such as mobile computing devices, mobile communication devices, set-top box television client devices, etc.

Although an example processing system has been described in FIG. 20, implementations of the subject matter and the functional operations described in this specification can be implemented in other types of digital electronic circuitry, or in computer software, firmware, or hardware, including the structures disclosed in this specification and their structural equivalents, or in combinations of one or more of them.

This specification uses the term “configured” in connection with systems and computer program components. For a system of one or more computers to be configured to perform particular operations or actions means that the system has installed on it software, firmware, hardware, or a combination of them that in operation cause the system to perform the operations or actions. For one or more computer programs to be configured to perform particular operations or actions means that the one or more programs include instructions that, when executed by data processing apparatus, cause the apparatus to perform the operations or actions.

Embodiments of the subject matter and the functional operations described in this specification can be implemented in digital electronic circuitry, in tangibly-embodied computer software or firmware, in computer hardware, including the structures disclosed in this specification and their structural equivalents, or in combinations of one or more of them. Embodiments of the subject matter described in this specification can be implemented as one or more computer programs, i.e., one or more modules of computer program instructions encoded on a tangible non transitory storage medium for execution by, or to control the operation of, data processing apparatus. The computer storage medium can be a machine-readable storage device, a machine-readable storage substrate, a random or serial access memory device, or a combination of one or more of them. Alternatively or in addition, the program instructions can be encoded on an artificially generated propagated signal, e.g., a machine-generated electrical, optical, or electromagnetic signal that is generated to encode information for transmission to suitable receiver apparatus for execution by a data processing apparatus.

The term “data processing apparatus” refers to data processing hardware and encompasses all kinds of apparatus, devices, and machines for processing data, including by way of example a programmable processor, a computer, or multiple processors or computers. The apparatus can also be, or further include, special purpose logic circuitry, e.g., an FPGA (field programmable gate array) or an ASIC (application specific integrated circuit). The apparatus can optionally include, in addition to hardware, code that creates an execution environment for computer programs, e.g., code that constitutes processor firmware, a protocol stack, a database management system, an operating system, or a combination of one or more of them.

A computer program, which may also be referred to or described as a program, software, a software application, an app, a module, a software module, a script, or code, can be written in any form of programming language, including compiled or interpreted languages, or declarative or procedural languages, and it can be deployed in any form, including as a stand alone program or as a module, component, subroutine, or other unit suitable for use in a computing environment. A program may, but need not, correspond to a file in a file system. A program can be stored in a portion of a file that holds other programs or data, e.g., one or more scripts stored in a markup language document, in a single file dedicated to the program in question, or in multiple coordinated files, e.g., files that store one or more modules, sub programs, or portions of code. A computer program can be deployed to be executed on one computer or on multiple computers that are located at one site or distributed across multiple sites and interconnected by a data communication network.

In this specification the term “engine” is used broadly to refer to a software-based system, subsystem, or process that is programmed to perform one or more specific functions. Generally, an engine will be implemented as one or more software modules or components, installed on one or more computers in one or more locations. In some cases, one or more computers will be dedicated to a particular engine, in other cases, multiple engines can be installed and running on the same computer or computers.

The processes and logic flows described in this specification can be performed by one or more programmable computers executing one or more computer programs to perform functions by operating on input data and generating output. The processes and logic flows can also be performed by special purpose logic circuitry, e.g., an FPGA or an ASIC, or by a combination of special purpose logic circuitry and one or more programmed computers.

Computers suitable for the execution of a computer program can be based on general or special purpose microprocessors or both, or any other kind of central processing unit. Generally, a central processing unit will receive instructions and data from a read only memory or a random access memory or both. The essential elements of a computer are a central processing unit for performing or executing instructions and one or more memory devices for storing instructions and data. The central processing unit and the memory can be supplemented by, or incorporated in, special purpose logic circuitry. Generally, a computer will also include, or be operatively coupled to receive data from or transfer data to, or both, one or more mass storage devices for storing data, e.g., magnetic, magneto optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device, e.g., a universal serial bus (USB) flash drive, to name just a few.

Computer readable media suitable for storing computer program instructions and data include all forms of non volatile memory, media and memory devices, including by way of example semiconductor memory devices, e.g., EPROM, EEPROM, and flash memory devices; magnetic disks, e.g., internal hard disks or removable disks; magneto optical disks; and CD ROM and DVD-ROM disks.

To provide for interaction with a user, embodiments of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's device in response to requests received from the web browser. Also, a computer can interact with a user by sending text messages or other forms of message to a personal device, e.g., a smartphone that is running a messaging application, and receiving responsive messages from the user in return.

Data processing apparatus for implementing machine learning models can also include, for example, special-purpose hardware accelerator units for processing common and compute-intensive parts of machine learning training or production, i.e., inference, workloads.

Machine learning models can be implemented and deployed using a machine learning framework, e.g., a TensorFlow framework, a Microsoft Cognitive Toolkit framework, an Apache Singa framework, or an Apache MXNet framework.

Embodiments of the subject matter described in this specification can be implemented in a computing system that includes a back end component, e.g., as a data server, or that includes a middleware component, e.g., an application server, or that includes a front end component, e.g., a client computer having a graphical user interface, a web browser, or an app through which a user can interact with an implementation of the subject matter described in this specification, or any combination of one or more such back end, middleware, or front end components. The components of the system can be interconnected by any form or medium of digital data communication, e.g., a communication network. Examples of communication networks include a local area network (LAN) and a wide area network (WAN), e.g., the Internet.

The computing system can include clients and servers. A client and server are generally remote from each other and typically interact through a communication network. The relationship of client and server arises by virtue of computer programs running on the respective computers and having a client-server relationship to each other. In some embodiments, a server transmits data, e.g., an HTML page, to a user device, e.g., for purposes of displaying data to and receiving user input from a user interacting with the device, which acts as a client. Data generated at the user device, e.g., a result of the user interaction, can be received at the server from the device.

While this specification contains many specific implementation details, these should not be construed as limitations on the scope of any invention or on the scope of what may be claimed, but rather as descriptions of features that may be specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially be claimed as such, one or more features from a claimed combination can in some cases be excised from the combination, and the claimed combination may be directed to a subcombination or variation of a subcombination.

Similarly, while operations are depicted in the drawings and recited in the claims in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the embodiments described above should not be understood as requiring such separation in all embodiments, and it should be understood that the described program components and systems can generally be integrated together in a single software product or packaged into multiple software products.

### EXAMPLES

The invention is further described in the following examples, which do not limit the scope of the invention described in the claims.

The present disclosure provides, as examples of application of this novel approach, detection of genetic aberrations relating to various conditions such as genetic disorders (e.g., Spinal Muscular Atrophy (SMA) and Congenital Adrenal Hyperplasia (CAH)), hematological neoplasms (e.g., chronic myeloid leukemia (CML), acute myeloid leukemia (AML) and acute lymphoblastic leukemia (ALL)) and infections (e.g., chikungunya (CHIK), dengue (DEN), cytomegalovirus (CMV) and Epstein-Barr Virus (EBV)).

The detection of CNVs were validated against multiplex ligation dependent probe amplification (MLPA) and digital droplet PCR (ddPCR). Fusion transcripts and infections were validated against real time PCR assays and methylation abnormalities were validated against methylation sensitive MLPA (MS-MLPA).

### Exemplar Clinical Conditions and Corresponding Genetic Targets

A number of human genomic targets, within which CNVs, small nucleotide variations (SNVs), or translocations, associated with particular disease-associated phenotypes, were selected. Targets selected for CNVs and/or SNVs included loci representative in the CNVs like the CYP21A2 gene [associated with ˜30% cases of 21-Hydroxylase deficient congenital adrenal hyperplasia (21-OH CAH)] and the SMN1 gene [95-98% cases with spinal muscular atrophy (SMA)], For detection of translocations/fusion transcripts genomic targets selected included the BCR-ABL1 [t(9;22)(q34;q11.2)] major (p210), BCR-ABL1 minor (p190) and BCR-ABL1 micro (p230) translocations/fusions associated with chronic myeloid leukemia (CML), the AML1-ETO [t(8;21)(q22; q22)], CBFB-MYH11 [inv(16) (p13q22)] and PML-RARA [(15;17)(q22;q21)] fusion transcripts associated with acute myeloid leukemia (AML), the TEL-AML1 (ETV6-RUNX1) [t(12;21)(p13;q22)], MLL-AF4 [t(4;11)(q21;q23)], and E2A-PBX1 [t(1;19)(q23;p13)] translocations/fusions associated with acute lymphoid leukemia (ALL). Infectious agents selected for proof of principle demonstration were Chikungunya Virus (CHIK), Dengue Virus (DEN), Cytomegalovirus (CMV) and Epstein Barr Virus (EBV).

### Designing of Target Specific Sequences (TSS)

(a) SNVs for human genetic disorders: multiple regions in each genomic target were selected to design target specific sequences (TSSs) that corresponds to sequences present within the genomic targets. Sequence data from release GRCH38/hg38 of the reference genome assembly were used as the source. A pair of TSSs was designed targeting each of multiple regions at the genomic targets. These specific targets for each clinical condition were selected based on the literature search and open data sources. The number of targeted regions varied from a single site in exon 7 of the SMN1 gene (to differentiate from the 99% similar exon 7 of the SMN2 gene) to 6 targets in the CYP21A2 gene. The TSS pool for SMA also included TSSs for polymorphisms [g.27134T>G and g.27706-27707delAT] reported to be associated with silent SMA carriers or the “2+0” genotype i.e. presence of two SMN1 gene copies present in a cis state on a single chromosome. This “2+0” genotype in the case of SMA is consistent with the diagnosis of a silent SMA carrier and hence is important for genetic screening and counseling.

(b) For detection of translocations/fusions associated with hematological neoplasms, TSSs were constructed by modifying oligonucleotide sequences previously described in the literature [Gabert J, et al Leukemia. 2003 December; 17(12):2318-57].

(c) For infections (Chikungunya, Dengue, CMV and EBV), TSSs were designed using the Primer 3 (open source software) or Primer Express 2.0 (ABI).

Each TSS (for human genetic disorders) was designed to minimize the occurrence of known common SNVs at their 3′ end using NCBI's dbSNP database build 146 version as a reference. Multiple TSSs in each pool were checked for thermodynamic stability and cross-interactions using Oligo Analyzer (v1.0.3).

To enable accurate relative quantification of CNVs, few TSSs targeting reference loci (i.e. loci not associated with a specific disease phenotype and which are known to have stable copy numbers in the population) were also designed—hereforeward referred to as reference TSS (RTSS). A pool of RTSSs was designed and various combinations of those (ranging from 5-15 pairs) were used along with different TSSs based on empirical determination of compatibility.

Each sequence from a TSS (or RTSS) pair was coupled to a unique barcode [probe identification sequence (PIDS)] and one out of two common adapters depending on whether they are on the 5′end or 3′ end of the region of interest.

A representative diagram is depicted in FIGS. 3A-3D. Multiples PIDS were designed to have a Levenshtein distance of at least 2 nucleotides therebetween thereby making them tolerant to a pre-determined degree of sequencing errors.

This (PIDS) indexing system was designed to effectively multiplex a wide dynamic range of targets from as few a single target to >1000 targets or even more, if required, in a single sample.

Sample preparation can be carried out by conventional methods. Samples may include a variety of biological matrices including blood, bone marrow, cerebrospinal fluid, pleural fluid, etc. Samples may be collected in variety of containers and form factors including, but not limited to, EDTA tubes, Citrate tubes, dried blood spots, or urine stabilization formulations.

### Example 1: Testing for Spinal Muscular Atrophy (SMA) Using the Ligation-Based Embodiment of the Present Invention

### 1) Identifying Target Specific Sequences (TSSs) to Create Target Specific Probes (TSPs):

Targets of interest: SMN1 (exons 7 and 8) and SMN2 (exon 7) and the ‘2+0’ single nucleotide markers in Intron 7 and between exon 7 and exon 8 in SMN1.

### Reference Controls: OCA2, KLKB, IL4, SETX, PARD3, HIPK3, AMOT, LAMA2, SPAST, PPHLNJ.

For each target and reference control, a pair of TSPs (TSP1 and TSP2) immediately adjacent to each other (with no gap in between) are selected. The 5′ member of the pair constitutes the first target specific sequence (TSS1) whereas the 3′ member of the pair constitutes the second target specific sequence (TSS2).

TSP1 has the following elements:

From 5′ to 3′ direction (first Common Adapter or “CA1”)-(PIDS1)-(5′Target Specific Sequence-TSS1), where the CommonAdapter (CA1) can be the 3′ portion of the Illumina P5 Nextera Adapter sequence that enables sequencing of the PIDS1 and SIDS1 regions flanking it on either side, and TSP2 has the following elements (where RC stands for reverse complement):

From 5′ to 3′ direction 5′phos-(3′ TSS2)-(PIDS2-RC)-(second Common Adapter, “CA2” or “CA2-RC”), where the CA2-RC can be the reverse complement of 3′ portion of the Illumina P7 Forked Adapter sequence that enables sequencing of the PIDS2 and SIDS2 regions flanking it on either side.

TABLE 1
Exemplar TSP1 constructs

| Name | CommonAdaptor CA1 | | |
|---|---|---|---|
| (Target) | (Illumina Nextera P5) (27 nt) | PIDS1 | 5′TargetSpecificSequence-TSS1 |
| SMN1 | TCGTCGGCAGCGTCAGATGTGTATAAGAG | AGTTAACA | AACTTCCTTTATTTTCCTTACAGGGTTTC |
| exon 7 | ACAG (SEQ ID NO: 1) | (SEQ ID | (SEQ ID NO: 3) |
| | | NO: 2) | |
| SMN1 | TCGTCGGCAGCGTCAGATGTGTATAAGAG | TGCAGCAG | GTGCTGGCCTCCCACCCCCACCC |
| exon 8.1 | ACAG (SEQ ID NO: 1) | (SEQ ID | (SEQ ID NO: 5) |
| | | NO: 4) | |
| SMN2 | TCGTCGGCAGCGTCAGATGTGTATAAGAG | CTCTTCTA | GAGCACCTTCCTTCTTTTTGATTTTGTCT |
| exon 7 | ACAG (SEQ ID NO: 1) | (SEQ ID | A (SEQ ID NO: 7) |
| | | NO: 6) | |
| SMN1 | TCGTCGGCAGCGTCAGATGTGTATAAGAG | CTAGGACG | AACCTTTCAACTTTTTAACATCTGAACTT |
| intron 7 | ACAG (SEQ ID NO: 1) | (SEQ ID | TTTAAC (SEQ ID NO: 9) |
| | | NO: 8) | |
| SMN1 | TCGTCGGCAGCGTCAGATGTGTATAAGAG | TGTACGTC | CCAAATGCAATGTGAAATATTTTACTGGA |
| exon 8.2 | ACAG (SEQ ID NO: 1) | (SEQ ID | CTCT (SEQ ID NO: 11) |
| | | NO: 10) | |
| OCA2 | TCGTCGGCAGCGTCAGATGTGTATAAGAG | GACCAACA | GCTCAACCTTGATCCAAGACAAGTCCTGA |
| | ACAG (SEQ ID NO: 1) | (SEQ ID | TTGC (SEQ ID NO: 13) |
| | | NO: 12) | |
| KLKB | TCGTCGGCAGCGTCAGATGTGTATAAGAG | TGTGGCGA | CCAAATGCCCAATACTGCCAGATGAGGT |
| | ACAG (SEQ ID NO: 1) | (SEQ ID | (SEQ ID NO: 15) |
| | | NO: 14) | |
| IL4 | TCGTCGGCAGCGTCAGATGTGTATAAGAG | TCTCTCTA | GGACACAAGTGCGATATCACCTTACAGGA |
| | ACAG (SEQ ID NO: 1) | (SEQ ID | GATC (SEQ ID NO: 17) |
| | | NO: 16) | |
| SETX | TCGTCGGCAGCGTCAGATGTGTATAAGAG | AGAGGAGC | TGCGTAATGGGAAAACTGAGTGTTACCT |
| | ACAG (SEQ ID NO: 1) | (SEQ ID | (SEQ ID NO: 19) |
| | | NO: 18) | |
| PARD3 | TCGTCGGCAGCGTCAGATGTGTATAAGAG | ACTAACTC | GAGAGTCTGTATCCACAGCCAGTGATCAG |
| | ACAG (SEQ ID NO: 1) | (SEQ ID | CCTT (SEQ ID NO: 21) |
| | | NO: 20) | |
| HIPK3 | TCGTCGGCAGCGTCAGATGTGTATAAGAG | CACAATAG | GCATAGTTCACCAAGTCCCAGTGGGCTTA |
| | ACAG (SEQ ID NO: 1) | (SEQ ID | AATC (SEQ ID NO: 23) |
| | | NO: 22) | |
| AMOT | TCGTCGGCAGCGTCAGATGTGTATAAGAG | GATTGGCA | CAGACGAGAACCGGAACTTGAGGCAAGA |
| | ACAG (SEQ ID NO: 1) | (SEQ ID | (SEQ ID NO: 25) |
| | | NO: 24) | |
| LAMA2 | TCGTCGGCAGCGTCAGATGTGTATAAGAG | CAATTGGA | GCAAATTCGGACTCGATGCCAAGAATCC |
| | ACAG (SEQ ID NO: 1) | (SEQ ID | (SEQ ID NO: 27) |
| | | NO: 26) | |
| SPAST | TCGTCGGCAGCGTCAGATGTGTATAAGAG | TCGAAGTA | GTACAGTCTGCTGGAGATGACAGAGTACT |
| | ACAG (SEQ ID NO: 1) | (SEQ ID | TGTA (SEQ ID NO: 29) |
| | | NO: 28) | |
| PPHLN1 | TCGTCGGCAGCGTCAGATGTGTATAAGAG | GACTTCGC | GAAAAGGAACTTGCTGAGGCTGCAAGCA |
| | ACAG (SEQ ID NO: 1) | (SEQ ID | (SEQ ID NO: 31) |
| | | NO: 30) | |

TABLE 2
Exemplar TSP2 constructs (the 5′ end of the oligonucleotide is
phosphorylated to enable ligation of the hybridized oligonucleotides)

| Name | 3′TargetSpecificSequence- | | CommonAdapter-CA2-RC (Illumina |
|---|---|---|---|
| (Target) | TSS2 | PIDS2-RC | P7 Forked Adapter-RC) (34 nt) |
| SMN1 | AGACAAAATCAAAAAGAAGGAAGGT | CTTGGCCA | AGATCGGAAGAGCACACCTTCTGAACTCCA |
| exon 7 | GCTGACATTCCTTAAATT | (SEQ ID | GTCAC (SEQ ID NO: 34) |
| | (SEQ ID NO: 32) | NO: 33) | |
| SMN1 | CACTCTT7TACAGATGGTTTTTCA | TCTGATCA | AGATCGGAAGAGCACACGTCTGAACTCCAG |
| exon 8.1 | (SEQ ID NO: 35) | (SEQ ID | TCAC (SEQ ID NO: 34) |
| | | NO: 36) | |
| SMN2 | AAACCCTGTAAGCAAAATAAAGGAA | CAGGTTCT | AGATCGGAAGAGCACACGrCTGAACTCCAG |
| exon 7 | GTAAAAA (SEQ ID NO: 37) | (SEQ ID | TCAC (SEQ ID NO: 34) |
| | | NO: 38) | |
| SMN1 | TGTTCAAAAACATTTGTTTCCACAA | TCATATGT | AGATCGGAAGAGCACACGTCTGAACTCCAG |
| intron 7 | ACCATAAAGTTTTAC (SEQ ID | (SEQ ID | TCAC (SEQ ID NO: 34) |
| | NO: 39) | NO: 40) | |
| SMN1 | TTTGAAAAACCATCTGTAAAAGACT | CACCGGTC | AGATCGGAAGAGCACACGTCTGAACTCCAG |
| exon 8.2 | (SEQ ID NO: 41) | (SEQ ID | TCAC (SEQ ID NO: 34) |
| | | NO: 42) | |
| OCA2 | AGAAGTGATCTTCACAAACATTGGA | GTTGCAAG | AGATCGGAAGAGCACACGTCPGAACTCCAG |
| | GGAGCTGC (SEQ ID NO: 43) | (SEQ ID | TCAC (SEQ ID NO: 34) |
| | | NO: 44) | |
| KLKB | GCACATTCCACCCAAGGTCTTTGCT | TGTGATAG | AGATCGGAAGAGCACACGTCTGAACTCCAG |
| | AT (SEQ ID NO: 45) | (SEQ ID | TCAC (SEQ ID NO: 34) |
| | | NO: 46) | |
| IL4 | ATCAAAACTTTGAACAGOCCACAGA | GAGGCCTG | AGATCGGAAGAGCACACGTCTGAACTCCAG |
| | GCAGAAG (SEQ ID NO: 47) | (SEQ ID | TCAC (SEQ ID NO: 34) |
| | | NO: 48) | |
| SETX | TTCCATCCAGACTCAAGAGAACTTT | AGAATTCA | AGATCGGAAGAGCACACGTCTGAACTCCAG |
| | CCGG (SEQ ID NO: 49) | (SEQ ID | TCAC (SEQ ID NO: 34) |
| | | NO: 50) | |
| PARD3 | CCCACTCTCTGGAGAGACAAATGAA | TGGCTGGA | AGATCGGAAGAGCACACGTCTGAACTCCAG |
| | TGGAAACC (SEQ ID NO: 51) | (SEQ ID | TCAC (SEQ ID NO: 34) |
| | | NO: 52) | |
| HIPK3 | CCCGTCTGTTACCATCCCCAACCAT | AGGAAGGC | AGATCGGAAGAGCACACGTCTGAACTCCAG |
| | TCATCAGA (SEQ ID NO: 53) | (SEQ ID | TCAC (SEQ ID NO: 34) |
| | | NO: 54) | |
| AHOT | GTTGGAAGGATGCTATGAGAAGGTG | ACATGCAG | AGATCGGAAGAGCACACGTCTGAACTCCAG |
| | GCA (SEQ ID NO: 55) | (SEQ ID | TCAC (SEQ ID NO: 34) |
| | | NO: 56) | |
| LAMA2 | ACTTGGCTGCAGGAGCTGCTATTGC | TCGAGACG | AGATCGGAAGAGCACACGTCTGAACTCCAG |
| | TTC (SEQ ID NO: 57) | (SEQ ID | TCAC (SEQ ID NO: 34) |
| | | NO: 58) | |
| SPAST | ATGGGTGCAACTAATAGGCCACAAG | AGTTGGTG | AGATCGGAAGAGCACACGTCTGAACTCCAG |
| | AGCTTGAT (SEQ ID NO: 59) | (SEQ ID | TCAC (SEQ ID NO: 34) |
| | | NO: 60) | |
| PPHLN1 | AGTGGGCTGCTGAAAAGCTAGAGAA | GAGGTTCA | AGATCGGAAGAGCACACGTCTGAACTCCAG |
| | ATC (SEQ ID NO: 61) | (SEQ ID | TCAC (SEQ ID NO: 34) |
| | | NO: 62) | |

2) Creation of an Assay Specific (i.e. SMA-Specific) Oligonucleotide Pool:

Custom synthesized oligo probes were ordered from custom oligonucleotide synthesis providers such as IDT from the sequences listed above. Lyophilized oligonucleotides were reconstituted to a final concentration of 100 uM (micromolar) using Tris-EDTA Buffer (10 mM Tris pH 8.0, 1 mM EDTA). 0.8 uL (microliters) of each oligonucleotide (TSP1 and TSP2 for each target and reference) were pooled and the volume is made up to 600 microliters such that the final concentration of each oligonucleotide is 133 nanoMolar (nM). This is treated as a 100× stock. The final concentration of each oligo in the 1× pool is 1.33 nanomolar.

3) Hybridization of Oligonucleotide Pool with Sample:

5 uL of genomic DNA (≥1 ng/uL) is denatured at 98 C for 5 min. 1.5 uL of the 1× oligo pool is mixed with 1.5 uL of hybridization buffer (1.5M KCl, 300 mM Tris-HCL pH 9.0, 1 mM EDTA, 12% PEG-6000, 10 mM DTT) and added to 5 uL of genomic DNA. After thorough mixing, the mix is denatured at 95° C. for 1 min, and subsequently incubated at 60° C. for 22 hours.

### 4) Ligation:

1.25 units AmpLigase (Epicentre/Lucigen) in 20 mM Tris-HCL pH 8.3, 25 mM KCl, 10 mM MgCl2, 0.5 mM NAD and 0.01% Triton X-100—is thoroughly mixed while ensuring that all reagents and samples are at 45° C. Ligation is carried out for 15 mins at 45° C. after which the reaction is terminated by heating to 98° C. for 10 minutes.

### 5) Incorporation of Sample Barcodes (SIDS) by PCR:

Sequences that enable tethering of constructs to the flow cell of the barcoding of individual samples are incorporated through PCR with unique custom synthesized oligonucleotides. A unique pair of oligonucleotides—SOA (first PCR primer) and SOB (second PCR primer)—are used per sample. The oligonucleotides have the following structures:

SOA (first PCR Primer): The SOA is of the format:
(SequencingInstrumentSpecificTetheringAdapter1-TA1)-(SID S1)-(CommonAdapter1-CA1), where the TA1 can be the Illumina P5 Binding Adapter and where CommonAdapter1 may be the 5′ portion of the Illumina Nextera P5 Sequencing

### Primer.

TABLE 3
Exemplar SOA (first PCR primer) sequence

| Name | TA1 (25 nt) | SIDS1 | CA1 (14 nt) |
|---|---|---|---|
| SOA-N | AATGATACGGCGACC | XXXXXXXXXXXX | TCGTCGGCAGC |
| | ACCGAGATCTACAC | (SEQ ID NO: | GTC (SEQ ID |
| | (SEQ ID NO: 63) | 64) | NO: 65) |

where XXXXXXXXXXXX (SEQ ID NO:64) is a molecular barcode with lengths between 8 and 12 (can be more or less depending on the multiplexing required). A list of example barcodes is in appendix A. Criteria for selection of these barcodes from the pool of possible barcodes are:
Inter-barcode edit distance: The barcode pool is chosen such that each barcode in the pool is separated by an edit distance (Levenshtein) of 2 or more from any other barcode in the pool.
Hairpin structure evaluation: each barcode is evaluated for possible hairpin structures and only those where hairpin structures do not exist or have a melting temperature less than 0° C. are selected.
Interaction with SOB: Each complete SOA construct is evaluated for possible hybridization with each SOB structure. Only construct pairs which display no significant hybridization structures at 60° C. are selected.
   SOB (second PCR primer): The SOB is of the format:
   (SequencingInstrumentSpecificTetheringAdapter2-TA2)-(SIDS2)-(CommonAdapter2-CA2), where TA2 can be the Illumina P7 Binding Adapter and where CA2 can be the 5′ portion of the Illumina P7 forked adapter sequencing primer

TABLE 4
Exemplar SOB (second PCR primer) sequence

| Name | TA2 (24 nt) | SIDS2 | CA2 (19 nt) |
|---|---|---|---|
| SOB-M | CAAGCAGAAGACGGC | YYYYYYYYYYYY | GTGACTGGAGTT |
| | ATACGAGAT (SEQ | (SEQ ID NO: | CAGACGT (SEQ |
| | ID NO: 66) | 67) | ID NO: 68) |

where YYYYYYYYYYYY (SEQ ID NO:67) is a molecular barcode with lengths between 8 and 12 (can be more or less depending on the multiplexing required). A list of example barcodes is in appendix B. Criteria for selection of these barcodes are similar to those set out for SOA—the same pool of barcodes can be used for both.

Unique pairs of oligonucleotides (one species of SOA and one species of SOB) at a final concentration of 400 nM are combined with the samples for the PCR. Commercial PCR master mixes such as Kapa HiFi Hotstart or Qiagen Quantitect Master Mix are used. The cycling conditions are initial denaturation at 95 C for 15 min, followed by 30 cycles of 95 C for 30 sec, 68 C for 45 sec and 72 C for 1 min 30 sec.

### 6) Pooling of Samples and Clean-Up:

The PCR products are quantified by a fluorometric assay (e.g. Thermo Qubit) and pooled at equimolar concentrations. The pool is purified using AMPure XP SPRI (solid phase reversible immobilization) technology. Alternative purification approaches which will be obvious to practitioners skilled in the art such as gel-based concentration, centrifugal spin column concentration, alcohol-salt precipitation, exonuclease and alkaline phosphatase treatment, may also be used in the concentration/clean-up steps.

7) Quantification of the Amplicon Pool by Fluorometry and qPCR:

The purified library is quantified using fluorometric quantification method and molarity is corrected using qPCR with quantification standards by an approach that routinely used by individuals skilled in the art.

### 8) Library Preparation and Loading of NGS:

The library is prepared and loaded onto the NGS according to standard published Illumina protocols which are known to practitioners skilled in the art. It is to be noted that the NGS platform being used is merely a method for readout, and alternative NGS platforms such as the Ion Torrent/Proton systems from ABI/Thermo and other systems from Roche, Qiagen, Pacific Biosystems, Oxford Nanopore, etc. may be used as well. In such cases, the adapters and tethering sequencing can be varied to ensure compatibility with the chosen sequencer platform which should be obvious to individuals who are familiar with those systems.

### 9) Probe Identification and Counting by NGS (Illumina SBS Chemistry)

A description for the steps using the Illumina Sequencer family. As indicated above, the invention can be readily adapted to other sequencing platforms.

### (a) Sequencer Configuration:

After dilution to a concentration appropriate to individual members of the Illumina sequencer family (e.g. iSeq or MiniSeq or MiSeq or NextSeq or HiSeq or NovaSeq) the pooled PCR products are captured within the sequencer instrument on the flow cell by the P5 and P7 tethering sequences (TA1 and/or TA2) at the ends of the construct. Each captured PCR product is clonally amplified to a cluster on the flow cell using the bridge PCR. Sequencing is initiated from the P5 end with the cluster tethered to the flow cell from the P7 end. Halfway through the cycle the molecule is flipped over and sequencing resumes from the P7 end with the cluster being anchored from the P5 end.
Read 1: The Illumina sequencer will read the amplicons generated from the ligated and amplified oligonucleotide constructs starting from the PIDS1 barcode. The sequencer is configured to read only the length of PIDS1 barcode (e.g. if the PIDS1 barcode is 12 bases long, there will be a 12 cycle Read 1)
Indexing Read 1: The Illumina sequencer will read the sample specific barcode SIDS1 in the SOA region (XXXXXXXXXXXX)(SEQ ID NO:64) and
Indexing Read 2: The barcode SIDS2 in the SOB region (YYYYYYYYYYYY) (SEQ ID NO:67) as part of its “Indexing cycles”. The sequencer reads only the number of bases specified in the barcode.
Read 2: The Illumina sequencer will read the amplicons generated from the ligated and amplified oligonucleotide constructs starting from the PIDS2 barcode. The sequencer is configured to read only the length of PIDS2 barcode (e.g. if the PIDS2 barcode is 12 bases long, there will be 12 cycle Read 2)

This sequencer configuration of 12 (Read 1)+12 (Indexing Read 1)+12 (Indexing Read 2)+12 (Read 2)=48 total cycles allows for rapid sequencing; e.g. an Illumina NextSeq sequencer will complete this protocol in less than 6 hours. Further, the configuration of SIDS1/SIDS2 allows for multiplexing of large number of samples. On an Illumina NextSeq sequencer with a cluster capacity of 400 Million, more than 6000 samples can be multiplexed with an average of 4000 reads for each of the 15 constructs (6000 samples*15 constructs/sample*4000 reads/construct=400 Million reads).

### (b) Demultiplexing Sample Specific Read Sequences:

The Illumina bcl2fastq software is configured with a SampleSheet.csv specifying the SIDS1/SIDS2 barcodes and upon execution, it demultiplexes reads corresponding to each unique pair of SIDS1/SIDS2.

### (c) Quality Filtering:

Each read is filtered such that the quality score for all bases in Read1/Read2 used to identify the read is above 30 on the phred scale (i.e. probability of base read being wrong is less than 1 in 1000).

### (d) Assigning and Counting Reads Corresponding to Oligonucleotide Constructs:

A custom software program is setup with a trie of all TSP barcodes (PIDS1 and PIDS2). In addition, all barcodes (derived by artificially inserting/deleting/substituting bases) within an edit distance of 2 from these barcodes are inserted into the trie as well. The leaf nodes of this trie structure stores information on the corresponding TSP. For each sample, the software walks the trie with the Read1/Read2 sequence. If both Read1 and Read2 are present in the trie and correspond to the same TSP sequence, the count for that TSP sequence for the sample is incremented. Once constructed, the trie is read-only and can be shared across multiple threads/processors to rapidly process millions of reads. On an Intel i5-2310M CPU@ 2.5 GHz processor with four cores, 5 million reads can be processed in 1 minute. The 400 million reads from a NextSeq run can be processed within 1.5 hrs. With a more capable processor (more cores, higher CPU frequency), this can be sped up further (to less than 30 minutes).

### (e) Calculating Copy Numbers:

Copy numbers are calculated by intra-sample normalization, Averaging per-TSP in Control Samples and Inter-sample normalization:
(e1) Intra-sample normalization by total number of reads: For each sample, the read counts for each construct are normalized by the total number of reads for the sample yielding a number from 0.0 to 1.0.
(e2) Averaging per-TSP in Control Samples: Across all control samples, the normalized values for each TSP from step (e1) are weighted by the number of known copies in the control sample and averaged.
(e3) Inter-sample normalization for each sample: For each TSP, the normalized value from step (e1) is divided by the per-TSP average normalized value from step (e2) to yield the ratio/copy number.

### 10) Interpretation and Reporting:

The ratios from the normalization algorithm are used to categorize the samples:
i) a value between 0.8-1.2 is interpreted as normal diploid, whereas
ii) a value >0.3 and <0.80 is interpreted as a heterozygous deletion and
iii) a value >1.3 and <1.75 is interpreted as a heterozygous duplication; a value >1.75 is interpreted as >3 copies.
iv) a value <0.1 is interpreted as a homozygous deletion.

### Example 2: Validation Study Performed on SMA Blinded Samples with the Ligation Embodiment of the Present Invention

Spinal muscular atrophy (SMA) is one of the most common autosomal recessive disorders associated with progressive degeneration of anterior horn cells of the spinal cord. Clinical signs and symptoms range from infantile onset severe hypotonia with severe morbidity and/or mortality to late onset mild to moderate proximal muscle weakness. The estimated incidence of SMA is 1:10000 live births and carrier frequency ranges from 1:40-1:70 in various populations. Treatment is mainly supportive and preventive (by prenatal diagnosis). Usually carriers are identified after one child with SMA is born in the family. In families with one child affected with SMA, both parents are obligate heterozygous carriers. The risk of recurrence in such families is 25%. Prenatal diagnosis for SMA is usually offered in each subsequent pregnancy of mother to prevent the recurrence. Owing to high carrier frequency in all populations, disease severity, availability of highly sensitive and specific molecular techniques capable detecting affected individuals and carriers, the American College of Medical Genetics and Genomics (ACMG) recommends population-based carrier screening. In case both partners are detected as carriers, subsequent prenatal diagnosis during pregnancy can prevent the birth of an affected child and drastically reduce the disease incidence.

The disorder is caused by homozygous deletions of exon 7 and 8 of the SMN1 gene in 95-98% of the cases. The remaining 2-5% cases are caused by small sequence variants in the SMN1 gene. The SMN1 gene is located on chromosome 5q13 region with closely situated highly homologous SMN2 gene.

All SMA carriers harbor only one functional copy of SMN1 gene, which is caused by a heterozygous deletion of exon 7 and 8 of SMN1 gene in 92-95% of the cases. In about 3.3-8.5% of individuals in various studied populations carry two copies of SMN1 gene on single chromosome in a cis state with “zero” or no copy on the other chromosome. This phenomenon is also known as “2+0” genotype and individuals with “2+0” genotype are referred to as silent SMA carriers. The gold standard for diagnosing heterozygous deletion in SMN1 gene is multiplex ligation dependent probe amplification (MLPA). Few other molecular techniques like quantitative PCR (qPCR) and digital droplet PCR (ddPCR) however, have been utilized in population-based screening for SMA carriers. The limitations of these techniques include scalability challenges for application to mass scale screening, need for manual interpretation, labor intensiveness, inability of some of the approaches to identify the “2+0” genotype and cost-effectiveness.

This example demonstrates application of the present invention in a single step platform for the identification of affected individuals harboring biallelic SMN1 gene exon 7 deletion and heterozygous carriers caused by SMN1 gene deletion as well as individuals harboring the “2+0” genotype who are at high risk of being silent SMA carriers in the clinical cohort. The validation study was done on 80 samples in a blinded manner. The results of the validation study were compared with the gold standard MLPA assay using SALSA MLPA Kit P060 (MRC-Holland, Amsterdam, Netherlands).

### Methodology

Reference DNA standards with known copies in the SMN1 and SMN2 genes were obtained from the NIGMS Human Genetic Cell Repository at the Coriell Institute for Medical Research. [Reference IDs were HG01773, HG02051, HG02882, NA00232, NA003815, GM19235, NA19984 and NA20294]. The concentration and quality of the DNA (260/280 nm ratio) was determined using the Nanodrop spectrophotometric system. All DNA samples with DNA concentration of 1 ng/uL (total 5 ng) were used for subsequent downstream processing. Briefly, the protocol involves hybridization of the sample DNA with assay specific pool of Target specific probes (for specific targets in the SMN1 and SMN2 genes) coupled with unique sequences (PIDS). For the SMN1 gene, four specific regions were targeted. Out of them, two oligonucleotides were targeted to single nucleotide variations (SNVs) in exon 7 and exon 8 differentiating it from the SMN2 gene. The most important site is c.840 C in exon 7 of the SMN1 gene. The presence of the alternate allele “T” in the SMN2 gene at this position results in skipping of the functionally relevant exon 7 in the SMN2 transcript. Another SNV in the SMN1 gene that differentiates it from the SMN2 gene is g.27734G>A in the 3′ UTR region (historically identified as exon 8) of the SMN1 gene. For detection of haplotypes associated with “2+0” genotypes, the two SNVs targeted were g.27134T>G (intron 7 of the SMN1 gene) and g.27706-27707delAT (inside the conventional exon 8 of the SMN1 gene). Additionally, 10 pairs of reference TSSs (RTSSs), targeting unlinked human genomic loci, were also used, which acted as controls for intra- and inter-sample normalizations. A second round of indexing was performed using PCR, leading to incorporation of sample specific unique barcodes (SIDS). Short-read paired-end sequencing using next generation sequencing (NGS) with Illumina's Sequencing by Synthesis Chemistry and analysis was performed using a pipeline according to the present invention, as described in the detailed description. Copy numbers of the SMN1 and SMN2 genes and the presence/absence of SNVs associated with the “2+0” genotypes were interpreted as described in the detailed description. A value between 0.8-1.2 is interpreted as normal diploid, whereas a value >0.3 and <0.80 is interpreted as a heterozygous deletion and a value <0.1 is interpreted as a homozygous deletion. Furthermore a value >1.3 and <1.75 is interpreted as a heterozygous duplication and a value >1.75 is interpreted as >3 copies.

Clinical interpretation

For SMA, the presence of more than 2 copies of exon 7 of the SMN1 gene is interpreted as a very low risk of being an SMA carrier and hence partner screening was not recommended. Identification of homozygous deletions of exon 7 of the SMN1 gene is consistent with a diagnosis of SMA, whereas detection of heterozygous deletions of exon 7 of the SMN1 gene are consistent with the individual being a heterozygous carrier for SMA; in such cases partner screening was recommended. In cases where two copies of exon 7 of the SMN1 gene were present, the number of raw NGS reads, for both polymorphisms (g.27134T>G and g.27706-27707delAT) associated with the “2+0” genotype, were counted. If raw NGS read counts for any of these polymorphisms were present beyond a minimum established threshold, then the sample was classified as one with an “increased risk of being a silent SMA carrier” and hence partner screening was recommended. In case none of these polymorphisms were detected in the sample, it was assigned to the category of “low risk of being an SMA carrier” and partner screening was not recommended. Using a semi-automated software pipeline, the results were binned as follows: (i) homozygous deletions of exon 7 of the SMN1 gene→affected with SMA, (ii) heterozygous deletions of exon 7 of the SMN1 gene→carriers for SMN1 gene deletion/SMA carriers and (iii) presence of the “2+0”-associated polymorphisms in a background of normal SMN1 copy numbers→likely to be silent SMA carriers and (iv) normal diploid copy numbers of SMN1→normal/low residual risk for being SMA carriers.

### Results

The blinded validation study included 80 clinically characterized samples and 8 reference standards. Eighteen samples (22.5%) showed the presence of homozygous deletions in the SMN1 gene. Thirty-six samples (45%) harbored two copies of the SMN1 gene and did not exhibit polymorphisms associated with the “2+0” genotype; hence they were categorized as “low residual risk of being SMA carriers”. Twenty-one (26.2%) samples harbored heterozygous deletions of the SMN1 gene and hence were labelled as SMA carriers. Heterozygous duplications of the SMN1 gene were present in five (6.25%) samples. For the SMN2 gene, 39 samples (48.75%) harbored the normal diploid complement, 22 samples (27.5%) harbored heterozygous deletions, 16 samples (20%) harbored heterozygous duplications, 2 samples (2.5%) harbored homozygous deletions and only one sample harbored a homozygous duplication. The representative results from different categories are represented in the table below.

TABLE 5
Detection of SMN1 and SMN2 gene copies in the
blinded validation study

| SMN1 genotype | Number | SMN2 genotype | Number |
|---|---|---|---|
| Diploid (normal) | 36 | Diploid (normal) | 39 |
| Heterozygous deletion | 21 | Heterozygous | 22 |
| (SMA carrier) | | deletion | |
| Homozygous deletion | 18 | Homozygous | 2 |
| (confirmed SMA case) | | deletion | |
| Heterozygous duplication of | 3 | Heterozygous | 16 |
| exon 7 and exon 8 | | duplication | |
| Heterozygous duplication of only | 2 | Homozygous | 1 |
| exon 7 (exon 8 was normal) | | duplication | |
| Total | 80 | | 80 |

The results correlated with those of MLPA for all 80 samples providing 100% positive and negative correlation. None of the samples harbored any of the high-risk polymorphisms associated with the “2+0” genotype.

### Discussion

The conventional molecular techniques used in the identification of affected SMA cases with homozygous deletions include polymerase chain reaction (PCR) and gel electrophoresis, restriction fragment length polymorphism (RFLP) analysis, quantitative real time PCR and MLPA. The in one instance, the present invention combines the power of techniques like qPCR and MLPA with Next Generation Sequencing (NGS) to simultaneously interrogate small nucleotide variations, copy number variations and methylation status at multiple sites across the genome. In some aspects, the present invention can be highly flexible with respect to the number of targets ranging from a single target in a single gene to multiple targets in a single gene or multiple targets in multiple genes. In addition, this technology is highly scalable; the architecture can enable multiplexing of thousands of samples in a single run and is only limited by the capacity of the sequencer and the multiplexing indices available. Many NGS-based bioinformatic pipelines have been developed to simultaneously detect copy number variations. However, to the best of our knowledge, none of these techniques are based on ultra-short read dual indexing system. Furthermore, it is possible to multiplex up to 10,000 samples in a single experiment for single or multiple targets. Moreover, the present technology is suitable to detect a dynamic range of copy number variations (small scale CNV i.e 1 vs 2 or 3 and large-scale variations in the mixed infection and neoplasms etc. Owing to the high degree of sequence homology between the SMN1 and SMN2 genes, most of the short-reads generated in capture-based NGS are difficult to assign definitively to one of the two genes. The advantages of the present invention over existing NGS based pipelines include ultra-short read sequencing, huge multiplexing capability and use of a semiautomated pipeline.

NGS is usually considered to be expensive owing to large initial set-up cost and the need of proprietary reagents. The proprietary laboratory and bioinformatics algorithms and unique barcoding system in the present invention obviates the need for batching samples, thereby making it cost effective for population-based screening. Furthermore, samples being analyzed for distinct conditions may be tested simultaneously. Additional sets of genomic targets relevant for specific populations can be added to an existing assay without a huge increase in cost.

### Example 3: Testing for CYP21A2-Associated CAH Using the Ligation-Based Embodiment of the Present Invention

### 1) Identifying Target Specific Sequences (TSSs) to Create Target Specific Probes (TSPs):

Targets of interest: The genes CYP21A2 and CYP21A1P.

### Reference Controls: OCA2, KLKB, IL4, SETX, PARD3, HIPK3, AMOT, LAMA2, SPAST, PPHLN1.

For each target and reference control, a pair of target specific probes (containing TSS and RTSS respectively), which are immediately adjacent to each other (with no gap in between), were selected. The 5′ member of the pair constitutes the first target specific sequence (TSS1) whereas the 3′ member of the pair constitutes the second target specific sequence (TSS2).

TSP1 has the following elements:
From 5′ to 3′ direction (CA1)-(PIDS1)-(5′TSS1), where the first Common Adapter (CA1) can be the 3′ portion of the Illumina P5 Nextera Adapter sequence that enables sequencing of the PIDS1 and SIDS1 regions flanking it on either side, and
TSP2 has the following elements (where RC stands for reverse complement):
From 5′ to 3′ direction 5′phos-(3′ TSS2)-(PIDS2-RC)-(CommonAdapter-CA2-RC), where the second CommonAdapter (CA2 or CA2-RC) can be the reverse complement of 3′ portion of the Illumina P7 Forked Adapter sequence that enables sequencing of the PIDS2 and SIDS2 regions flanking it on either side.
The TSP1 constructs are as follows:

TABLE 6
Exemplar TSP1 sequences

| | CommonAdapter CA1 (Illumina | | 5′TargetSpecificSequence- |
|---|---|---|---|
| Name (Target) | Nextera P5) (27 nt) | PIDS1 | TSS1 |
| CYP21A2_EX6 | TCGTCGGCAGCGTCAGATGTGTATAAG | CAACGTTC | GAAGCAGGCCATAGAGAAGAGGGAT |
| | AGACAG (SEQ ID NO: 1) | (SEQ ID | CACAT (SEQ ID NO: 70) |
| | | NO: 69) | |
| CYP21A2_EX3 | TCGTCGGCAGCGTCAGATGTGTATAAG | CAGCTGAG | TCTAGGAACTACCCGGACCTGTCCT |
| | AGACAG (SEQ ID NO: 1) | (SEQ ID | TGG (SEQ ID NO: 72) |
| | | NO: 71) | |
| CYP21A2_I2G | TCGTCGGCAGCGTCAGATGTGTATAAG | CTCTCTCG | CACCAGCTTGTCTGCAGGAGGAGG |
| | AGACAG (SEQ ID NO: 1) | (SEQ ID | (SEQ ID NO: 74 |
| | | NO: 73) | |
| CYP21A2_I2G_A | TCGTCGGCAGCGTCAGATGTGTATAAG | AGAGAGAT | CACCAGCTTGTCTGCAGGAGGAGA |
| | AGACAG (SEQ ID NO: 1) | (SEQ ID | (SEQ ID NO: 76) |
| | | NO: 75) | |
| CYP21A2_INT6 | TCGTCGGCAGCGTCAGATGTGTATAAG | GAGAAGAT | CCGAGGGGAGGCCGTCCACGT |
| | AGACAG (SEQ ID NO: 1) | (SEQ ID | (SEQ ID NO: 78) |
| | | NO: 77) | |
| CYP21A2_EX4 | TCGTCGGCAGCGTCAGATGTGTATAAG | CACAGCGA | GAATTCTCTCTCCTCACCTGCAGCA |
| | AGACAG (SEQ ID NO: 1) | (SEQ ID | TCAT (SEQ ID NO: 80) |
| | | NO: 79) | |
| CYP21A2 | TCGTCGGCAGCGTCAGATGTGTATAAG | TCAGGATA | CAGAGCTCCCTTCCTGACCCTCCGC |
| 3UTRT1 | AGACAG (SEQ ID NO: 1) | (SEQ ID | C (SEQ ID NO: 82) |
| | | NO: 81) | |
| CYP2lAP_EX6 | TCGTCGGCAGCGTCAGATGTGTATAAG | TCGGTTAC | GAAGCAGGCCATAGAGAAGAGGGAT |
| | AGACAG (SEQ ID NO: 1) | (SEQ ID | CACAA (SEQ ID NO: 84) |
| | | NO: 83) | |
| CYP21AP_EX3 | TCGTCGGCAGCGTCAGATGTGTATAAG | TGCGTATC | TCTAGGAACTACCCGGACCTGTCCT |
| | AGACAG (SEQ ID NO: 1) | (SEQ ID | TGA (SEQ ID NO: 86) |
| | | NO: 85) | |
| CYP21A2_I2G_C | TCGTCGGCAGCGTCAGATGTGTATAAG | ACCGGTTC | CACCAGCTTGTCTGCAGGAGGAGC |
| | AGACAG (SEQ ID NO: 1) | (SEQ ID | (SEQ ID NO: 88) |
| | | NO: 87) | |
| CYP21AP_INT6 | TCGTCGGCAGCGTCAGATGTGTATAAG | ACTGTGAG | CCGAGGGGAGGCCGTCCACGC |
| | AGACAG (SEQ ID NO: 1) | (SEQ ID | (SEQ ID NO: 90) |
| | | NO: 89) | |
| CYP21AP_EX4 | TCGTCGGCAGCGTCAGATGTGTATAAG | TCTCCTCG | GAATTCTCTCTCCTCACCTGCAGCA |
| | AGACAG (SEQ ID NO: 1) | (SEQ ID | TCAA (SEQ ID NO: 92) |
| | | NO: 91) | |
| CYP21AP | TCGTCGGCAGCGTCAGATGTGTATAAG | AGAGAGAT | CAGAGCTCCCTTCCTGACCCTCCGC |
| 3UTRT1 | AGACAG (SEQ ID NO: 1) | (SEQ ID | T (SEQ ID NO: 94) |
| | | NO: 75) | |
| OCA2 | TCGTCGGCAGCGTCAGATGTGTATAAG | GACCAACA | GCTCAACCTTGATCCAAGACAAGTC |
| | AGACAG (SEQ ID NO: 1) | (SEQ ID | CTGATTGC (SEQ ID NO: 13) |
| | | NO: 12) | |
| KLKB | TCGTCGGCAGCGTCAGATGTGTATAAG | TGTGGCGA | CCAAATGCCCAATACTGCCAGATGA |
| | AGACAG (SEQ ID NO: 1) | (SEQ ID | GGT (SEQ ID NO: 15) |
| | | NO: 14) | |
| IL4 | TCGTCGGCAGCGTCAGATGTGTATAAG | TCTCTCTA | GGACACAAGTGCGATATCACCTTAC |
| | AGACAG (SEQ ID NO: 1) | (SEQ ID | AGGAGATC (SEQ ID NO: 17) |
| | | NO: 16) | |
| SETX | TCGTCGGCAGCGTCAGATGTGTATAAG | AGAGGAGC | TGCGTAATGGGAAAACTGAGTGTTA |
| | AGACAG (SEQ ID NO: 1) | (SEQ ID | CCT (SEQ ID NO: 19) |
| | | NO: 18) | |
| PARD3 | TCGTCGGCAGCGTCAGATGTGTATAAG | ACTAACTC | GAGAGTCTGTATCCACAGCCAGTGA |
| | AGACAG (SEQ ID NO: 1) | (SEQ ID | TCAGCCTT (SEQ ID NO: 21) |
| | | NO: 20) | |
| HIPK3 | TCGTCGGCAGCGTCAGATGTGTATAAG | CACAATAG | GCATAGTTCACCAAGTCCCAGTGGG |
| | AGACAG (SEQ ID NO: 1) | (SEQ ID | CTTAAATC (SEQ ID NO: 23) |
| | | NO: 22) | |
| AMOT | TCGTCGGCAGCGTCAGATGTGTATAAG | GATTGGCA | CAGACGAGAACCGGAACTTGAGGCA |
| | AGACAG (SEQ ID NO: 1) | (SEQ ID | AGA (SEQ ID NO: 25) |
| | | NO: 24) | |
| LAMA2 | TCGTCGGCAGCGTCAGATGTGTATAAG | CAATTGGA | GCAAATTCGGACTCGATGCCAAGAA |
| | AGACAG (SEQ ID NO: 1) | (SEQ ID | TCC (SEQ ID NO: 27) |
| | | NO: 26) | |
| SPAST | TCGTCGGCAGCGTCAGATGTGTATAAG | TCGAAGTA | GTACAGTCTGCTGGAGATGACAGAG |
| | AGACAG (SEQ ID NO: 1) | (SEQ ID | TACTTGTA (SEQ ID NO: 29) |
| | | NO: 28) | |
| PPHLN1 | TCGTCGGCAGCGTCAGATGTGTATAAG | GACTTCGC | GAAAAGGAACTTGCTGAGGCTGCAA |
| | AGACAG (SEQ ID NO: 1) | (SEQ ID | GCA (SEQ ID NO: 31) |
| | | NO: 30) | |

The TSP2 constructs are as follows (where the 5′ end of the oligonucleotide is phosphorylated):

TABLE 7
Exemplar TSP2 sequences

| | 3′TargetSpecificSequence- | | CommonAdapter-CA2-RC (Illumina |
|---|---|---|---|
| Name (Target) | TSS2 | PIDS2-RC | P7 Forked Adapter-RC) (34 nt) |
| CYP21A2_EX6 | CGTGGAGATGCAGCTGAGGCAGCAC | GATAGCAT | AGATCGGAAGAGCACACGTCTGAACTCCAG |
| | AA (SEQ ID NO: 95) | (SEQ ID | TCAC (SEQ ID NO: 34) |
| | | NO: 96) | |
| CYP21A2_EX3 | GAGACTACTCCCTGCTCTGGAAAGC | AACCAGGT | AGATCGGAAGAGCACACGTCTGAACTCCAG |
| | CCACAA (SEQ ID NO: 97) | (SEQ ID | TCAC (SEQ ID NO: 34) |
| | | NO: 98) | |
| CYP21A2_I2G | TGGGGGCTGGAGGGTGGGAACT | CATGACTA | AGATCGGAAGAGCACACGTCTGAACTCCAG |
| | (SEQ ID NO: 99) | (SEQ ID | TCAC (SEQ ID NO: 34) |
| | | NO: 100) | |
| CYP21A2_I2G_C | TGGGGGCTGGAGGGTGGGAACT | | AGATCGGAAGAGCACACGTCTGAACTCCAG |
| | (SEQ ID NO: 99) | | TCAC (SEQ ID NO: 34) |
| CYP21A2_INT6 | ACAGTCCCCACCTTGTGCTGCCTCA | GTCTCGGA | AGATCGGAAGAGCACACGTCTGAACTCCAG |
| | (SEQ ID NO: 101) | (SEQ ID | TCAC (SEQ ID NO: 34) |
| | | NO: 102) | |
| CYP21A2_EX4 | CTGTTACCTCACCTTCGGAGACAAG | CTCTAAGT | AGATCGGAAGAGCACACGTCTGAACTCCAG |
| | ATCAAG (SEQ ID NO: 103) | (SEQ ID | TCAC (SEQ ID NO: 34) |
| | | NO: 104) | |
| CYP21A2 | GCAGAGGATTGAGGCTTAATTCTGA | CATCGTGT | AGATCGGAAGAGCACACGTCTGAACTCCAG |
| 3UTRT1 | GCTGG (SEQ ID NO: 105) | (SEQ ID | TCAC (SEQ ID NO: 34) |
| | | NO: 106) | |
| CYP21AP_EX6 | CGTGGAGATGCAGCTGAGGCAGCAC | GCAACCTT | AGATCGGAAGAGCACACGTCTGAACTCCAG |
| | AA (SEQ ID NO: 95) | (SEQ ID | TCAC (SEQ ID NO: 34) |
| | | NO: 107) | |
| CYP21AP_EX3 | GAGACTACTCCCTGCTCTGGAAAGC | GAGATTCT | AGATCGGAAGAGCACACGTCTGAACTCCAG |
| | CCACAA (SEQ ID NO: 97) | (SEQ ID | TCAC (SEQ ID NO: 34) |
| | | NO: 108) | |
| CYP21AP_I2G | TGGGGGCTGGAGGGTGGGAACT | CACTGCTT | AGATCGGAAGAGCACACGTCTGAACTCCAG |
| | (SEQ ID NO: 99) | (SEQ ID | TCAC (SEQ ID NO: 34) |
| | | NO: 109) | |
| CYP21AP_INT6 | ACAGTCCCCACCTTGTGCTGCCTCA | AGGTACGA | AGATCGGAAGAGCACACGTCTGAACTCCAG |
| | (SEQ ID NO: 101) | (SEQ ID | TCAC (SEQ ID NO: 34) |
| | | NO: 110) | |
| CYP21AP_EX4 | CTGTTACCTCACCTTCGGAGACAAG | ACCGAGTC | AGATCGGAAGAGCACACGTCTGAACTCCAG |
| | ATCAAG (SEQ ID NO: 103) | (SEQ ID | TCAC (SEQ ID NO: 34) |
| | | NO: l11) | |
| CYP21AP | GCAGAGGATTGAGGCTTAATTCTGA | CACAAGTA | AGATCGGAAGAGCACACGTCTGAACTCCAG |
| 3UTRT1 | GCTGG (SEQ ID NO: 105 | (SEQ ID | TCAC (SEQ ID NO: 34) |
| | | NO: 112) | |
| OCA2 | AGAAGTGATCTTCACAAACATTGGA | GTTGCAAG | AGATCGGAAGAGCACACGTCTGAACTCCAG |
| | GGAGCTGC (SEQ ID NO: 43) | (SEQ ID | TCAC (SEQ ID NO: 34) |
| | | NO: 44) | |
| KLKB | GCACATTCCACCCAAGGTGTTTGCT | TGTGATAG | AGATCGGAAGAGCACACGTCTGAACTCCAG |
| | ATT (SEQ ID NO: 45) | (SEQ ID | TCAC (SEQ ID NO: 34) |
| | | NO: 46) | |
| IL4 | ATCAAAACTTTGAACAGCCTCACAG | GAGGCCTG | AGATCGGAAGAGCACACGTCTGAACTCCAG |
| | AGCAGAAG (SEQ ID NO: 47) | (SEQ ID | TCAC (SEQ ID NO: 34) |
| | | NO: 48) | |
| SETX | TTCCATCCAGACTCAAGAGAACTTT | AGAATTCA | AGATCGGAAGAGCACACGTCTGAACTCCAG |
| | CCGG (SEQ ID NO: 49) | (SEQ ID | TCAC (SEQ ID NO: 34) |
| | | NO: 50) | |
| PARD3 | CCCACTCTCTGGAGAGACAAATGAA | TGGCTGGA | AGATCGGAAGAGCACACGTCTGAACTCCAG |
| | TGGAAACC (SEQ ID NO: 51) | (SEQ ID | TCAC (SEQ ID NO: 34) |
| | | NO: 52) | |
| HIPK3 | CCCGTCTGTTACCATCCCCAACCAT | AGGAAGGC | AGATCGGAAGAGCACACGTCTGAACTCCAG |
| | TCATCAGA (SEQ ID NO: 53) | (SEQ ID | TCAC (SEQ ID NO: 34) |
| | | NO: 54) | |
| AMOT | GTTGGAAGGATGCTATGAGAAGGTG | ACATGCAG | AGATCGGAAGAGCACACGTCTGAACTCCAG |
| | GCA (SEQ ID NO: 55) | (SEQ ID | TCAC (SEQ ID NO: 34) |
| | | NO: 56) | |
| LAMA2 | ACTTGGCTGCAGCAGCTGCTATTGC | TCGAGACG | AGATCGGAAGAGCACACGTCTGAACTCCAG |
| | TTC (SEQ ID NO: 57) | (SEQ ID | TCAC (SEQ ID NO: 34) |
| | | NO: 58) | |
| SPAST | ATGGGTGCAACTAATAGGCCACAAG | AGTTGGTG | AGATCGGAAGAGCACACGTCTGAACTCCAG |
| | AGCTTGAT (SEQ ID NO: 59) | (SEQ ID | TCAC (SEQ ID NO: 34) |
| | | NO: 60 | |
| PPHLN1 | AGTGGGCTGCTGAAAAGCTAGAGAA | GAGGTTCA | AGATCGGAAGAGCACACGTCTGAACTCCAG |
| | ATC (SEQ ID NO: 61) | (SEQ ID | TCAC (SEQ ID NO: 34) |
| | | NO: 62) | |

### 2) Creation of Assay Specific Oligonucleotide Pools:

Custom synthesized oligos are ordered from custom oligonucleotide synthesis providers such as IDT from the sequences listed above. Lyophilized oligonucleotides are reconstituted to a final concentration of 100 uM (micromolar) using Tris-EDTA Buffer (10 mM Tris pH 8.0, 1 mM EDTA). 0.8 uL (microliters) of each oligonucleotide (TSP1 and TSP2 for each target and reference) are pooled and the volume is made up to 600 microliters such that the final concentration of each oligonucleotide is 133 nanoMolar (nM). This is treated as a 100× stock. The final concentration of each oligo in the 1× pool is 1.33 nanomolar.

3) Hybridization of Oligonucleotide Pool with Sample:

5 uL of genomic DNA (at >1 ng/uL) is denatured at 98 C for 5 min. 1.5 uL of the 1× oligo pool is mixed with 1.5 uL of hybridization buffer (1.5M KCl, 300 mM Tris-HCL pH 9.0, 1 mM EDTA, 12% PEG-6000, 10 mM DTT) and added to 5 uL of genomic DNA. After thorough mixing, the mix is denatured at 95° C. for 1 min, and subsequently incubated at 60° C. for 22 hours.

### 4) Ligation

1.25 units AmpLigase (Epicentre/Illumina/Lucigen) in 20 mM Tris-HCL pH 8.3, 25 mM KCl, 10 mM MgCl2, 0.5 mM NAD and 0.01% Triton X-100—is thoroughly mixed while ensuring that all reagents and samples are at 45° C. Ligation is carried out for 15 mins at 45° C. after which the reaction is terminated by heating to 98° C. for 10 minutes.

### 5) Incorporation of Sample Barcodes (SIDS) by PCR:

Sequences that enable tethering of constructs to the flow cell of the and barcoding of individual samples are incorporated through PCR with unique custom synthesized oligonucleotides. A unique pair of oligonucleotides—SOA and SOB—are used per sample. The oligonucleotides have the following structures:

SOA (first PCR primer)
The SOA is of the format:
(SequencingInstrumentSpecificTetheringAdapter1-TA1)-(SIDS1)-(CommonAdapter1-CA1), where the TA1 can be the Illumina P5 Binding Adapter and where CommonAdapter1 may be the 5′ portion of the Illumina Nextera P5 Sequencing Primer

TABLE 8
Exemplar SOA (first PCR primer) sequence

| Name | TA1 (25 nt) | SIDS1 | CA1 (14 nt) |
|---|---|---|---|
| SOA-N | AATGATACGGCGACC | XXXXXXXXXXXX | TCGTCGGCAGC |
| | ACCGAGATCTACAC | (SEQ ID | GTC (SEQ ID |
| | (SEQ ID NO: 63) | NO: 64) | NO: 65) |

where XXXXXXXXXXXX (SEQ ID NO:64) is a molecular barcode with lengths between 8 and 12 (can be more or less depending on the multiplexing required). A list of example barcodes is in appendix A. Criteria for selection of these barcodes from the pool of possible barcodes are:
Inter-barcode edit distance: The barcode pool is chosen such that each barcode in the pool is separated by an edit distance (Levenshtein) of 2 or more from any other barcode in the pool.
Hairpin structure evaluation: each barcode is evaluated for possible hairpin structures and only those where hairpin structures do not exist or have a melting temperature less than 0° C. are selected.
Interaction with SOB: Each complete SOA construct is evaluated for possible hybridization with each SOB structure. Only construct pairs which display no significant hybridization structures at 60 C are selected.

### SOB

The SOB is of the format:
(SequencingInstrumentSpecificTetheringAdapter2-TA2)-(SIDS2)-(CommonAdapter2-CA2), where TA2 can be the Illumina P7 Binding Adapter and where CA2 can be the 5′ portion of the Illumina P7 forked adapter sequencing primer.

TABLE 9
Exemplar SOB (second PCR primer) sequence

| Name | TA2 (24 nt) | SIDS2 | CA2 (19 nt) |
|---|---|---|---|
| SOB-M | CAAGCAGAAGACGGC | YYYYYYYYYYYY | GTGACTGGAGTTCA |
| | ATACGAGAT (SEQ | (SEQ ID | GACGT (SEQ ID |
| | ID NO: 66) | NO: 67) | NO: 68) |

where YYYYYYYYYYYY (SEQ ID NO:67) is a molecular barcode with lengths between 8 and 12 (can be more or less depending on the multiplexing required). A list of example barcodes is in appendix B. Criteria for selection of these barcodes are similar to those set out for SOA—the same pool of barcodes can be used for both.

Unique pairs of oligonucleotides (one species of SOA and one species of SOB) at a final concentration of 400 nM are combined with the samples for the PCR. Commercial PCR master mixes such as Kapa HiFi Hotstart or Qiagen Quantitect Master Mix are used. The cycling conditions are initial denaturation at 95 C for 15 min, followed by 30 cycles of 95 C for 30 sec, 68 C for 45 sec and 72 C for 1 min 30 sec.

### 6) Pooling of Samples and Clean-Up:

The PCR products are quantified by a fluorometric assay (e.g. Thermo Qubit) and pooled at equimolar concentrations. The pool is purified using AMPure XP SPRI (solid phase reversible immobilization) technology. Alternative purification approaches which will be obvious to practitioners skilled in the art such as gel-based concentration, centrifugal spin column concentration, alcohol-salt precipitation, exonuclease and alkaline phosphatase treatment, etc. may also be used in the concentration/clean-up steps.

7) Quantification of Pool by Fluorometry and qPCR:

The purified library is quantified using fluorometric quantification method and molarity is corrected using qPCR with quantification standards.

8) Library Preparation and Loading of NGS: The Library is Prepared and Loaded onto the NGS According to Standard Illumina Protocol.

### 9) Probe Identification and Counting by NGS (Illumina SBS Chemistry)

The prepared library is sequenced in an Illumina sequencer by methods readily apparent to anyone skilled in the art.
a) Sequencer configuration: The PCR products are captured on the flow cell by the P5 and P7 tethering sequences at the ends of the construct. Each captured PCR product is clonally amplified to a cluster on the flow cell using the bridge PCR. Sequencing is initiated from the P5 end with the cluster tethered to the flow cell from the P7 end. Halfway through the cycle the molecule is flipped over and sequencing resumes from the P7 end with the cluster being anchored from the P5 end.
i) Read 1: The Illumina sequencer will read the amplicons generated from the ligated probes starting from the PIDS1 barcode. The sequencer is configured to read only the length of PIDS1 barcode (e.g. if the PIDS1 barcode is 12 bases long, there will be a 12 cycle Read 1)
ii) Indexing Read 1: Indexing cycles: The Illumina sequencer will read the sample specific barcode SIDS1 in the SOA region (XXXXXXXXXXXX)(SEQ ID NO:64) and
iii) Indexing Read 2: The barcode SIDS2 in the SOB region (YYYYYYYYYYYY) (SEQ ID NO:67) as part of its “Indexing cycles”. The sequencer reads only the number of bases specified in the barcode.
iv) Read 2: The Illumina sequencer will read the amplicons generated from the ligated probes starting from the PIDS2 barcode. The sequencer is configured to read only the length of PIDS2 barcode (e.g. if the PIDS2 barcode is 12 bases long, there will be 12 cycle Read 2)

This sequencer configuration of 12 (Read 1)+12 (Indexing Read 1)+12 (Indexing Read 2)+12 (Read 2)=48 total cycles allows for rapid sequencing; an Illumina NextSeq sequencer will complete this protocol in less than 6 hours. Further, the configuration of SIDS1/SIDS2 allows for multiplexing of large number of samples. On an Illumina NextSeq sequencer with a cluster capacity of 400 Million, more than 6000 samples can be multiplexed with an average of 4000 reads for each of the 15 constructs (6000 samples*15 constructs/sample*4000 reads/construct=400 Million reads)

### b) Demultiplexing Sample Specific Read Sequences:

The Illumina bcl2fastq software is configured with a SampleSheet.csv specifying the SIDS1/SIDS2 barcodes and upon execution, it demultiplexes reads corresponding to each unique pair of SIDS1/SIDS2.

### c) Quality Filter:

Each read is filtered such that the quality score for all bases in Read1/Read2 used to identify the read is above 30 on the phred scale (i.e. probability of base read being wrong is 1 in 1000).

### d) Assigning and Counting Reads Corresponding to Probes:

A custom software program is setup with a trie of all TSP barcodes (PIDS1 and PIDS2). In addition, all barcodes (derived by artificially inserting/deleting/substituting bases) within an edit distance of 2 from these barcodes are inserted into the trie as well. The leaf nodes of this trie structure stores information on the corresponding TSP.

For each sample:
i) For each read, the software walks the trie with the Read1/Read2 sequence. If both Read1 and Read2 are present in the trie and correspond to the same TSP sequence, the count for that TSP sequence for the sample is incremented.

Once constructed, the trie is read-only and can be shared across multiple threads/processors to rapidly process millions of reads. On an Intel i5-2310M CPU@ 2.5 GHz processor with four cores, 5 million reads can be processed in 1 minute. The 400 million reads from a NextSeq run can be processed within 1.5 hrs. With a more capable processor (more cores, higher CPU frequency), this can be sped up further (to less than 30 minutes).

### (e) Calculating Copy Numbers:

Copy numbers are calculated by intra-sample normalization, Averaging per-TSP in Control Samples and Inter-sample normalization:
(e1) Intra-sample normalization by total number of reads: For each sample, the read counts for each construct are normalized by the total number of reads for the sample yielding a number from 0.0 to 1.0.
(e2) Averaging per-TSP in Control Samples: Across all control samples, the normalized values for each TSP from step (e1) are weighted by the number of known copies in the control sample and averaged.
(e3) Inter-sample normalization for each sample: For each TSP, the normalized value from step (e1) is divided by the per-TSP average normalized value from step (e2) to yield the ratio/copy number.

### 10) Interpretation and Reporting:

The ratios from the normalization algorithm are used to categorize the samples:
i) a value between 0.8-1.2 is interpreted as normal diploid, whereas
ii) a value >0.3 and <0.80 is interpreted as a heterozygous deletion and
iii) a value >1.3 and <1.75 is interpreted as a heterozygous duplication; a value >1.75 is interpreted as >3 copies.
iv) a value <0.1 is interpreted as a homozygous deletion.

Homozygous deletions of ≥2 TSPs targeting the CYP21A2 gene are interpreted as homozygous deletions or large gene rearrangements or gene conversions. These findings are consistent with the diagnosis of CYP21A2-associated CAH. Homozygous deletions of one TSP targeting of CYP21A2 gene is suggestive of, but not confirmatory of CYP21A2-associated CAH.

### Example 4: Testing for Spinal Muscular Atrophy (SMA) Using the Extension-Ligation Based Embodiment of the Present Invention

### 1) Identifying Target Specific Sequences (TSSs) to Create Target Specific Oligonucleotides (TSPs):

Targets of interest: SMN1 Exon 7 and SMN2 Exon 8

### Reference Controls: RNaseP, TERT and CFTR

For each target and reference control, a pair of target specific oligonucleotides are selected. The 5′ member of the pair constitutes the first target specific sequence (TSS1) whereas the 3′ member of the pair constitutes the second target specific sequence (TSS2).
TSP1 has the following elements:
From 5′ to 3′ direction (CommonAdapter-CA1)-(PIDS1)-(5′TargetSpecificSequence-TSS1)
Where the CommonAdapter (CA1) can be the 3′ portion of the Illumina P5 Nextera Adapter sequence that enables sequencing of the PIDS1 and SIDS1 regions flanking it on either side, and
The TSP1 constructs are as follows:

TABLE 10
Exemplar TSP1 sequences

| Name | CommonAdapter CA1 (Illumina | | 5′TargetSpecificSequence- |
|---|---|---|---|
| (Target) | Nextera P5) (27 nt) | PIDS1 | TSS1 |
| SMN1 | TCGTCGGCAGCGTCAGATGTGTATAAG | TCAACCAC | CCTTCCTTCTTTTTGATTTTGTCAG |
| exon 7 | AGACAG (SEQ ID NO: 1) | (SEQ ID | (SEQ ID NO: 114) |
| | | NO: 113) | |
| SMN2 | TCGTCGGCAGCGTCAGATGTGTATAAG | AGCTCTGC | CCTTCCTTCTTTTTGATTTTGTCAA |
| exon 7 | AGACAG (SEQ ID NO: 1) | (SEQ ID | (SEQ ID NO: 116) |
| | | NO: 115) | |
| TERT | TCGTCGGCAGCGTCAGATGTGTATAAG | GTACCGTC | GGCACACGTGGCTTTTCG (SEQ |
| | AGACAG (SEQ ID NO: 1) | (SEQ ID | ID NO: 118) |
| | | NO: 117) | |
| CFTR | TCGTCGGCAGCGTCAGATGTGTATAAG | GTGCGCAG | AGCCGACACTTTGCTTGCTATG |
| | AGACAG (SEQ ID NO: 1) | (SEQ ID | (SEQ ID NO: 120) |
| | | NO: 119) | |
| RNaseP | TCGTCGGCAGCGTCAGATGTGTATAAG | TCCTTCCG | AGATTTGGACCTGCGAGCG (SEQ |
| | AGACAG (SEQ ID NO: 1) | (SEQ ID | ID NO: 122) |
| | | NO: 121) | |

TSP2 has the following elements (where RC stands for reverse complement): From 5′ to 3′ direction 5′phos-(3′TargetSpecificSequence-TSS2)-(PIDS2-RC)-(CommonAdapter-CA2-RC)
Where the CommonAdapter-CA2-RC can be the reverse complement of 3′ portion of the Illumina P7 Forked Adapter sequence that enables sequencing of the PIDS2 and SIDS2 regions flanking it on either side.
The TSP2 constructs are as follows (where the 5′ end of the oligonucleotide is phosphorylated):

TABLE 11
Exemplar TSP2 sequences

| Name | 3′TargetSpecificSequence- | | CommonAdapter-CA2-RC (Illumina |
|---|---|---|---|
| (Target) | TSS2 | PIDS2-RC | P7 Forked Adapter-RC) (34 nt) |
| SMN | AACCCTGTAAGGAAAATAAAGGAAG | ATTCTCCT | AGATCGGAAGAGCACACGTCTGAACTCCAG |
| common | (SEQ ID NO: 37) | (SEQ ID | TCAC (SEQ ID NO: 34) |
| | | NO: 123) | |
| TERT | TTGCATAAACTTACGAGGTTCACC | TCTGTGAC | AGATCGGAAGAGCACACGTCTGAACTCCAG |
| | (SEQ ID NO: 124) | (SEQ ID | TCAC (SEQ ID NO: 34) |
| | | NO: 125) | |
| CF17 | CATTCTGTTCTTCAAGCACCTATGT | TCATTGCA | AGATCGGAAGAGCACACGTCTGAACTCCAG |
| | C (SEQ ID NO: 126) | (SEQ ID | TCAC (SEQ ID NO: 34) |
| | | NO: 127) | |
| RNaseP | ACTTGTGGAGACAGCCGCTC (SEQ | GTGGCCAG | AGATCGGAAGAGCACACGTCTGAACTCCAG |
| | ID NO: 128) | (SEQ ID | TCAC (SEQ ID NO: 34) |
| | | NO: 129) | |

### 2) Creation of Assay Specific Oligonucleotide Pools:

Custom synthesized oligos are ordered from custom oligonucleotide synthesis providers such as IDT from the sequences listed above. Lyophilized oligonucleotides are reconstituted to a final concentration of 100 uM (micromolar) using Tris-EDTA Buffer (10 mM Tris pH 8.0, 1 mM EDTA). 0.8 uL (microliters) of each oligonucleotide (TSP1 and TSP2 for each target and reference) are pooled and the volume is made up to 600 microliters such that the final concentration of each oligonucleotide is 133 nanoMolar (nM). This is treated as a 100× stock. The final concentration of each oligo in the 1× pool is 1.33 nanomolar.
3) Hybridization of Oligonucleotide Pool with Sample:
5 uL of genomic DNA/cDNA is denatured at 98 C for 5 min. 1.5 uL of the 1× oligo pool is mixed with 1.5 uL of hybridization buffer (1.5M KCl, 300 mM Tris-HCL pH 9.0, 1 mM EDTA, 12% PEG-6000, 10 mM DTT) and added to 5 uL of genomic DNA. After thorough mixing, the mix is denatured at 95° C. for 1 min, and subsequently incubated at 60° C. for 22 hours.

### 4) Extension of TSP1:

The TSP1 is extended using a polymerase lacking or with minimal 5′ exonuclease activity and strand displacement activity such as the Q5 High-Fidelity DNA polymerase (NEB) or equivalent. Extension is carried out for 98° C. for 3 min, followed by incubation at 60° C. for 10 min

### 5) Ligation:

1.25 units AmpLigase (Epicentre/Illumina/Lucigen) in 20 mM Tris-HCL pH 8.3, 25 mM KCl, 10 mM MgCl2, 0.5 mM NAD and 0.01% Triton X-100 is thoroughly mixed while ensuring that all reagents and samples are at 45° C. Ligation is carried out for 15 mins at 45° C. after which the reaction is terminated by heating to 98° C. for 10 minutes.

### 6) Incorporation of Sample Barcodes (SIDS) by PCR:

Sequences that enable tethering of constructs to the flow cell of the and barcoding of individual samples are incorporated through PCR with unique custom synthesized oligonucleotides. A unique pair of oligonucleotides—SOA and SOB—are used per sample. The oligonucleotides have the following structures:
SOA (first PCR primer)
The SOA is of the format:
(SequencingInstrumentSpecificTetheringAdapter1-TA1)-(SIDS1)-(CommonAdapter1-CA1), where the TA1 can be the Illumina P5 Binding Adapter and where CommonAdapter1 may be the 5′ portion of the Illumina Nextera P5 Sequencing Primer

TABLE 12
Exemplar SOA (first PCR primer) sequence

| Name | TA1 (25 nt) | SIDS1 | CA1 (14 nt) |
|---|---|---|---|
| SOA-N | AATGATACGGCGACC | XXXXXXXXXXXX | TCGTCGGCAGC |
| | ACCGAGATCTACAC | (SEQ ID NO: | GTC (SEQ ID |
| | (SEQ ID NO: 63) | 64) | NO: 65) |

where XXXXXXXXXXXX (SEQ ID NO:64) is a molecular barcode with lengths between 8 and 12 (can be more or less depending on the multiplexing required). A list of example barcodes is in appendix A. Criteria for selection of these barcodes from the pool of possible barcodes are:
Inter-barcode edit distance: The barcode pool is chosen such that each barcode in the pool is separated by an edit distance (Levenshtein) of 2 or more from any other barcode in the pool.
Hairpin structure evaluation: each barcode is evaluated for possible hairpin structures and only those where hairpin structures do not exist or have a melting temperature less than 0° C. are selected.
Interaction with SOB: Each complete SOA construct is evaluated for possible hybridization with each SOB structure. Only construct pairs which display no significant hybridization structures at 60 C are selected.

### SOB

The SOB is of the format:
(SequencingInstrumentSpecificTetheringAdapter2-TA2)-(SIDS2)-(CommonAdapter2-CA2), where TA2 can be the Illumina P7 Binding Adapter and where CA2 can be the 5′ portion of the Illumina P7 forked adapter sequencing primer.

TABLE 13
Exemplar SOB (second PCR primer) sequence

| Name | TA2 (24 nt) | SIDS2 | CA2 (19 nt) |
|---|---|---|---|
| SOB-M | CAAGCAGAAGACGGC | YYYYYYYYYYYY | GTGACTGGAGTT |
| | ATACGAGAT (SEQ | (SEQ ID NO: | CAGACGT (SEQ |
| | ID NO: 66) | 67) | ID NO: 68) |

where YYYYYYYYYYYY (SEQ ID NO:67) is a molecular barcode with lengths between 8 and 12 (can be more or less depending on the multiplexing required). A list of example barcodes is in appendix B. Criteria for selection of these barcodes are similar to those set out for SOA—the same pool of barcodes can be used for both.

Unique pairs of oligonucleotides (one species of SOA and one species of SOB) at a final concentration of 400 nM are combined with the samples for the PCR. Commercial PCR master mixes such as Kapa HiFi Hotstart or Qiagen Quantitect Master Mix are used. The cycling conditions are initial denaturation at 95 C for 15 min, followed by 30 cycles of 95 C for 30 sec, 68 C for 45 sec and 72 C for 1 min 30 sec.

### 7) Pooling of Samples and Clean-Up:

The PCR products are quantified by a fluorometric assay (e.g. Thermo Qubit) and pooled at equimolar concentrations. The pool is purified using AMPure XP SPRI (solid phase reversible immobilization) technology. Alternative purification approaches which will be obvious to practitioners skilled in the art such as gel-based concentration, centrifugal spin column concentration, alcohol-salt precipitation, exonuclease and alkaline phosphatase treatment, etc. may also be used in the concentration/clean-up steps.

8) Quantification of Pool by Fluorometry and qPCR:

The purified library is quantified using fluorometric quantification method and molarity is corrected using qPCR with quantification standards.

9) Library Preparation and Loading of NGS: The Library is Prepared and Loaded onto the NGS According to Standard Illumina Protocol.

### 10) Probe Identification and Counting by NGS (Illumina SBS Chemistry)

The prepared library is sequenced in an Illumina sequencer by methods readily apparent to anyone skilled in the art.
a) Sequencer configuration: The PCR products are captured on the flow cell by the P5 and P7 tethering sequences at the ends of the construct. Each captured PCR product is clonally amplified to a cluster on the flow cell using the bridge PCR. Sequencing is initiated from the P5 end with the cluster tethered to the flow cell from the P7 end. Halfway through the cycle the molecule is flipped over and sequencing resumes from the P7 end with the cluster being anchored from the P5 end.
i) Read 1: The Illumina sequencer will read the amplicons generated from the ligated probes starting from the PIDS1 barcode. The sequencer is configured to read only the length of PIDS1 barcode (e.g. if the PIDS1 barcode is 12 bases long, there will be a 12 cycle Read 1)
ii) Indexing Read 1: Indexing cycles: The Illumina sequencer will read the sample specific barcode SIDS1 in the SOA region (XXXXXXXXXXXX)(SEQ ID NO:64) and
iii) Indexing Read 2: The barcode SIDS2 in the SOB region (YYYYYYYYYYYY) (SEQ ID NO:67) as part of its “Indexing cycles”. The sequencer reads only the number of bases specified in the barcode.
iv) Read 2: The Illumina sequencer will read the amplicons generated from the ligated probes starting from the PIDS2 barcode. The sequencer is configured to read only the length of PIDS2 barcode (e.g. if the PIDS2 barcode is 12 bases long, there will be 12 cycle Read 2)

This sequencer configuration of 12 (Read 1)+12 (Indexing Read 1)+12 (Indexing Read 2)+12 (Read 2)=48 total cycles allows for rapid sequencing; an Illumina NextSeq sequencer will complete this protocol in less than 6 hours. Further, the configuration of SIDS1/SIDS2 allows for multiplexing of large number of samples. On an Illumina NextSeq sequencer with a cluster capacity of 400 Million, more than 6000 samples can be multiplexed with an average of 4000 reads for each of the 15 constructs (6000 samples*15 constructs/sample*4000 reads/construct=400 Million reads).

### b) Demultiplexing Sample Specific Read Sequences:

The Illumina bcl2fastq software is configured with a SampleSheet.csv specifying the SIDS1/SIDS2 barcodes and upon execution, it demultiplexes reads corresponding to each unique pair of SIDS1/SIDS2.

### c) Quality Filter:

Each read is filtered such that the quality score for all bases in Read1/Read2 used to identify the read is above 30 on the phred scale (i.e. probability of base read being wrong is 1 in 1000).

### d) Assigning and Counting Reads Corresponding to Probes:

A custom software program is setup with a trie of all TSP barcodes (PIDS1 and PIDS2). In addition, all barcodes (derived by artificially inserting/deleting/substituting bases) within an edit distance of 2 from these barcodes are inserted into the trie as well. The leaf nodes of this trie structure stores information on the corresponding TSP.

For each sample:
i) For each read, the software walks the trie with the Read1/Read2 sequence. If both Read1 and Read2 are present in the trie and correspond to the same TSP sequence, the count for that TSP sequence for the sample is incremented.

Once constructed, the trie is read-only and can be shared across multiple threads/processors to rapidly process millions of reads. On an Intel i5-2310M CPU@ 2.5 GHz processor with four cores, 5 million reads can be processed in 1 minute. The 400 million reads from a NextSeq run can be processed within 1.5 hrs. With a more capable processor (more cores, higher CPU frequency), this can be sped up further (to less than 30 minutes).

### (e) Calculating Copy Numbers:

Copy numbers are calculated by intra-sample normalization, Averaging per-TSP in Control Samples and Inter-sample normalization:
(e1) Intra-sample normalization by total number of reads: For each sample, the read counts for each construct are normalized by the total number of reads for the sample yielding a number from 0.0 to 1.0.
(e2) Averaging per-TSP in Control Samples: Across all control samples, the normalized values for each TSP from step (e1) are weighted by the number of known copies in the control sample and averaged.
(e3) Inter-sample normalization for each sample: For each TSP, the normalized value from step (e1) is divided by the per-TSP average normalized value from step (e2) to yield the ratio/copy number.

### 11) Interpretation and Reporting:

The ratios from the normalization algorithm are used to categorize the samples:
i) a value between 0.8-1.2 is interpreted as normal diploid, whereas
ii) a value >0.3 and <0.80 is interpreted as a heterozygous deletion and
iii) a value >1.3 and <1.75 is interpreted as a heterozygous duplication; a value >1.75 is interpreted as >3 copies.
iv) a value <0.1 is interpreted as a homozygous deletion.

### Example 5: Testing for Chronic Myeloid Leukemia (CML) Using the Extension-Ligation Based Embodiment of the Present Invention

### 1) Identifying Target Specific Sequences (TSSs) to Create Target Specific Oligonucleotides (TSPs):

Targets of interest: BCR-ABL1 major (p210) fusion transcript
Reference Controls: GUS, B2M and ABL1 transcript
For each target and reference control, a pair of target specific oligonucleotides are selected. The 5′ member of the pair constitutes the first target specific sequence (TSS1) whereas the 3′ member of the pair constitutes the second target specific sequence (TSS2).

### TSP1 has the Following Elements:

From 5′ to 3′ direction (CommonAdapter-CA1)-(PIDS1)-(5′TargetSpecificSequence-TSS1)
Where the CommonAdapter (CA1) can be the 3′ portion of the Illumina P5 Nextera Adapter sequence that enables sequencing of the PIDS1 and SIDS1 regions flanking it on either side, and
The TSP1 constructs are as follows:

TABLE 14
Exemplar TSP1 sequences:

| Name | CommonAdapter CA1 (Illumina | | 5′TargetSpecificSequence- |
|---|---|---|---|
| (Target) | Nextera P5) (27 nt) | PIDS1 | TSS1 |
| BCR-ABL1 | TCGTCGGCAGCGTCAGATGTGTATAAG | ACTGTGAG | TCCGCTGACCATCAAYAAGGA |
| | AGACAG (SEQ ID NO: 1) | (SEQ ID | (SEQ ID NO: 130) |
| | | NO: 69) | |
| GUS | TCGTCGGCAGCGTCAGATGTGTATAAG | ACCGGTTC | GAAAATATGTGGTTGGAGAGCTCAT |
| | AGACAG (SEQ ID NO: 1) | (SEQ ID | T (SEQ ID NO: 131) |
| | | NO: 87) | |
| B2M | TCGTCGGCAGCGTCAGATGTGTATAAG | TTGATATA | GAGTATGCCTGCCGTGTG (SEQ |
| | AGACAG (SEQ ID NO: 1) | (SEQ ID | ID NO: 133) |
| | | NO: 132) | |
| ABL1 | TCGTCGGCAGCGTCAGATGTGTATAAG | AGCGATAT | TGGAGATAACACTCTAAGCATAACT |
| | AGACAG (SEQ ID NO: 1) | (SEQ ID | AAAGGT (SEQ ID NO: 135) |
| | | NO: 134) | |

TSP2 has the following elements (where RC stands for reverse complement): From 5′ to 3′ direction 5′phos-(3′TargetSpecificSequence-TSS2)-(PIDS2-RC)-(CommonAdapter-CA2-RC)
Where the CommonAdapter-CA2-RC can be the reverse complement of 3′ portion of the Illumina P7 Forked Adapter sequence that enables sequencing of the PIDS2 and SIDS2 regions flanking it on either side.
The TSP2 constructs are as follows (where the 5′ end of the oligonucleotide is phosphorylated):

TABLE 15
Exemplar TSP2 sequences

| Name | 3′TargetSpecificSequence- | | CommonAdapter-CA2-RC (Illumina |
|---|---|---|---|
| (Target) | TSS2 | PIDS2-RC | P7 Forked Adapter-RC) (34 nt) |
| BCR-ABL1 | PTGAGCCTCAGGGTCTGAGTG | ATCTCTCT | AGATCGGAAGAGCACACGTCTGAACTCCAG |
| | (SEQ ID NO: 136) | | TCAC (SEQ ID NO: 34) |
| GUS | PAAAAAGGGGATCTTCACTCGG | CGAGGAGA | AGATCGGAAGAGCACACGTCTGAACTCCAG |
| | (SEQ ID NO: 137) | | TCAC (SEQ ID NO: 34) |
| B2M | AGATGCCGCATTTGGATT | ACTGTAGG | AGATCGGAAGAGCACACGTCTGAACTCCAG |
| | (SEQ ID NO: 138) | | TCAC (SEQ ID NO: 34) |
| ABL1 | TGGGTCCCAAGCAACTACATC | ACTGTCAT | AGATCGGAAGAGCACACGTCTGAACTCCAG |
| | (SEQ ID NO: 139) | | TCAC (SEQ ID NO: 34) |

### 2) Creation of Assay Specific Oligonucleotide Pools:

Custom synthesized oligos are ordered from custom oligonucleotide synthesis providers such as IDT from the sequences listed above. Lyophilized oligonucleotides are reconstituted to a final concentration of 100 uM (micromolar) using Tris-EDTA Buffer (10 mM Tris pH 8.0, 1 mM EDTA). 0.8 uL (microliters) of each oligonucleotide (TSP1 and TSP2 for each target and reference) are pooled and the volume is made up to 600 microliters such that the final concentration of each oligonucleotide is 133 nanoMolar (nM). This is treated as a 100× stock. The final concentration of each oligo in the 1× pool is 1.33 nanomolar.
3) Hybridization of Oligonucleotide Pool with Sample:

5 uL of genomic DNA/cDNA is denatured at 98 C for 5 min. 1.5 uL of the 1× oligo pool is mixed with 1.5 uL of hybridization buffer (1.5M KCl, 300 mM Tris-HCL pH 9.0, 1 mM EDTA, 12% PEG-6000, 10 mM DTT) and added to 5 uL of genomic DNA. After thorough mixing, the mix is denatured at 95° C. for 1 min, and subsequently incubated at 60° C. for 22 hours. Alternatively the starting material may be RNA, which can be reverse transcribed to cDNA using methods that are known to individuals skilled in the art, such as random priming, priming with oligodT primers and priming with target specific primers.

### 4) Extension of TSP1:

The TSP1 is extended using a polymerase lacking or with minimal 5′ exonuclease activity and strand displacement activity such as the Q5 High-Fidelity DNA polymerase (NEB) or equivalent. Extension is carried out for 98° C. for 3 min, followed by incubation at 60° C. for 10 min

### 5) Ligation:

1.25 units AmpLigase (Epicentre/Illumina/Lucigen) in 20 mM Tris-HCL pH 8.3, 25 mM KCl, 10 mM MgCl2, 0.5 mM NAD and 0.01% Triton X-100 is thoroughly mixed while ensuring that all reagents and samples are at 45° C. Ligation is carried out for 15 mins at 45° C. after which the reaction is terminated by heating to 98° C. for 10 minutes.

### 6) Incorporation of Sample Barcodes (SIDS) by PCR:

Sequences that enable tethering of constructs to the flow cell of the and barcoding of individual samples are incorporated through PCR with unique custom synthesized oligonucleotides. A unique pair of oligonucleotides—SOA and SOB—are used per sample. The oligonucleotides have the following structures:
SOA (first PCR primer)
The SOA is of the format:
(SequencingInstrumentSpecificTetheringAdapter1-TA1)-(SIDS1)-(CommonAdapter1-CA1), where the TA1 can be the Illumina P5 Binding Adapter and where CommonAdapter1 may be the 5′ portion of the Illumina Nextera P5 Sequencing Primer

TABLE 16
Exemplar SOA (first PCR primer) sequence

| Name | TA1 (25 nt) | SIDS1 | CA1 (14 nt) |
|---|---|---|---|
| SOA-N | AATGATACGGCGACC | XXXXXXXXXX | TCGTCGGCAGCGTC |
| | ACCGAGATCTACAC | XX (SEQ ID | (SEQ ID NO: |
| | (SEQ ID NO: 63) | NO: 64) | 65) |

where XXXXXXXXXXXX (SEQ ID NO:64) is a molecular barcode with lengths between 8 and 12 (can be more or less depending on the multiplexing required). A list of example barcodes is in appendix A. Criteria for selection of these barcodes from the pool of possible barcodes are:
Inter-barcode edit distance: The barcode pool is chosen such that each barcode in the pool is separated by an edit distance (Levenshtein) of 2 or more from any other barcode in the pool.
Hairpin structure evaluation: each barcode is evaluated for possible hairpin structures and only those where hairpin structures do not exist or have a melting temperature less than 0° C. are selected.
Interaction with SOB: Each complete SOA construct is evaluated for possible hybridization with each SOB structure. Only construct pairs which display no significant hybridization structures at 60 C are selected.

### SOB

The SOB is of the format:
(SequencingInstrumentSpecificTetheringAdapter2-TA2)-(SIDS2)-(CommonAdapter2-CA2), where TA2 can be the Illumina P7 Binding Adapter and where CA2 can be the 5′ portion of the Illumina P7 forked adapter sequencing primer.

TABLE 17
Exemplar SOB (second PCR primer) sequence

| Name | TA2 (24 nt) | SIDS2 | CA2 (19 nt) |
|---|---|---|---|
| SOB-M | CAAGCAGAAGAC | YYYYYYYYYYYY | GTGACTGGAGTTC |
| | GGCATACGAGAT | (SEQ ID NO: | AGACGT |
| | (SEQ ID NO: 66) | 67) | (SEQ ID NO: 68) |

where YYYYYYYYYYYY (SEQ ID NO:67) is a molecular barcode with lengths between 8 and 12 (can be more or less depending on the multiplexing required). A list of example barcodes is in appendix B. Criteria for selection of these barcodes are similar to those set out for SOA—the same pool of barcodes can be used for both.

Unique pairs of oligonucleotides (one species of SOA and one species of SOB) at a final concentration of 400 nM are combined with the samples for the PCR. Commercial PCR master mixes such as Kapa HiFi Hotstart or Qiagen Quantitect Master Mix are used. The cycling conditions are initial denaturation at 95 C for 15 min, followed by 30 cycles of 95 C for 30 sec, 68 C for 45 sec and 72 C for 1 min 30 sec.

### 7) Pooling of Samples and Clean-Up:

The PCR products are quantified by a fluorometric assay (e.g. Thermo Qubit) and pooled at equimolar concentrations. The pool is purified using AMPure XP SPRI (solid phase reversible immobilization) technology. Alternative purification approaches which will be obvious to practitioners skilled in the art such as gel-based concentration, centrifugal spin column concentration, alcohol-salt precipitation, exonuclease and alkaline phosphatase treatment, etc. may also be used in the concentration/clean-up steps.

8) Quantification of Pool by Fluorometry and qPCR:

The purified library is quantified using fluorometric quantification method and molarity is corrected using qPCR with quantification standards.

9) Library Preparation and Loading of NGS: The Library is Prepared and Loaded onto the NGS According to Standard Illumina Protocol.

### 10) Probe Identification and Counting by NGS (Illumina SBS Chemistry)

The prepared library is sequenced in an Illumina sequencer by methods readily apparent to anyone skilled in the art.
a) Sequencer configuration: The PCR products are captured on the flow cell by the P5 and P7 tethering sequences at the ends of the construct. Each captured PCR product is clonally amplified to a cluster on the flow cell using the bridge PCR. Sequencing is initiated from the P5 end with the cluster tethered to the flow cell from the P7 end. Halfway through the cycle the molecule is flipped over and sequencing resumes from the P7 end with the cluster being anchored from the P5 end.
i) Read 1: The Illumina sequencer will read the amplicons generated from the ligated probes starting from the PIDS1 barcode. The sequencer is configured to read only the length of PIDS1 barcode (e.g. if the PIDS1 barcode is 12 bases long, there will be a 12 cycle Read 1)
ii) Indexing Read 1: Indexing cycles: The Illumina sequencer will read the sample specific barcode SIDS1 in the SOA region (XXXXXXXXXXXX)(SEQ ID NO:64) and
iii) Indexing Read 2: The barcode SIDS2 in the SOB region (YYYYYYYYYYYY) (SEQ ID NO:67) as part of its “Indexing cycles”. The sequencer reads only the number of bases specified in the barcode.
iv) Read 2: The Illumina sequencer will read the amplicons generated from the ligated probes starting from the PIDS2 barcode. The sequencer is configured to read only the length of PIDS2 barcode (e.g. if the PIDS2 barcode is 12 bases long, there will be 12 cycle Read 2)

This sequencer configuration of 12 (Read 1)+12 (Indexing Read 1)+12 (Indexing Read 2)+12 (Read 2)=48 total cycles allows for rapid sequencing; an Illumina NextSeq sequencer will complete this protocol in less than 6 hours. Further, the configuration of SIDS1/SIDS2 allows for multiplexing of large number of samples. On an Illumina NextSeq sequencer with a cluster capacity of 400 Million, more than 6000 samples can be multiplexed with an average of 4000 reads for each of the 15 constructs (6000 samples*15 constructs/sample*4000 reads/construct=400 Million reads).

### b) Demultiplexing Sample Specific Read Sequences:

The Illumina bcl2fastq software is configured with a SampleSheet.csv specifying the SIDS1/SIDS2 barcodes and upon execution, it demultiplexes reads corresponding to each unique pair of SIDS1/SIDS2.

### c) Quality Filter:

Each read is filtered such that the quality score for all bases in Read1/Read2 used to identify the read is above 30 on the phred scale (i.e. probability of base read being wrong is 1 in 1000).

### d) Assigning and Counting Reads Corresponding to Probes:

A custom software program is setup with a trie of all TSP barcodes (PIDS1 and PIDS2). In addition, all barcodes (derived by artificially inserting/deleting/substituting bases) within an edit distance of 2 from these barcodes are inserted into the trie as well. The leaf nodes of this trie structure stores information on the corresponding TSP.

For each sample:
i) For each read, the software walks the trie with the Read1/Read2 sequence. If both Read1 and Read2 are present in the trie and correspond to the same TSP sequence, the count for that TSP sequence for the sample is incremented.

Once constructed, the trie is read-only and can be shared across multiple threads/processors to rapidly process millions of reads. On an Intel i5-2310M CPU@ 2.5 GHz processor with four cores, 5 million reads can be processed in 1 minute. The 400 million reads from a NextSeq run can be processed within 1.5 hrs. With a more capable processor (more cores, higher CPU frequency), this can be sped up further (to less than 30 minutes).

### e) Calculating for the Presence of BCR-ABL Fusion Transcripts:

The raw NGS reads for GUS, B2M and ABL1 as well as BCR-ABL1 fusion transcripts are counted. If raw reads for BCR-ABL1 fusion transcripts are above a predetermined threshold value and the GUS, B2M and ABL1 counts are above empirically determined reference thresholds, the sample is interpreted as “positive” for chronic myeloid leukemia (CML). The relative quantitation is calculated as the ratio of raw NGS reads for BCR-ABL1 fusion transcripts and GUS, B2M and ABL1 transcripts.

### Example 6: Testing for Infectious Agents Such as Chikungunya, Dengue, Cytomegalo Virus (CMV), and Epstein-Barr Virus (EBV) Using the PCR-Based Embodiment of the Present Invention

### 1) Identifying Target Specific Sequences (TSSs) to Create Target Specific Probes (TSPs):

Targets of interest: Unique regions within the E1 envelope protein gene of Chikungunya virus, the 3′ UTR of Dengue virus, B2 glycoprotein of CMV, and a unique locus in the EBV genome between the BRRF2 and BKRF2 genes.

### Reference Controls: RNaseP.

For each target and reference control, a first primer and a second primer (a sense primer, called OligoA-In and an antisense primer, called OligoB-In, respectively) are designed targeting a unique region within the relevant genome.

OligoA-In (first primer) has the following elements:
From 5′ to 3′ direction (CommonAdapter-CA1)-(PIDS1)-(5′TargetSpecificSequence-TSS1), where the CommonAdapter (CA1) can be the 3′ portion of the Illumina P5 Nextera Adapter sequence that enables sequencing of the PIDS1 and SIDS1 regions flanking it on either side, and
The OligoA-In constructs are as follows:

TABLE 18
Exemplar OligoA-In (first primer) sequence

| Name | CommonAdapter CA1 | | 5′TargetSpecificSequence- |
|---|---|---|---|
| (Target) | (Illumina Nextera P5) (27 nt) | PIDS1 | TSS1 |
| ChikungunyaFw | TCGTCGGCAGCGTCAGATGTGTATAAGAGACAG | CGCAAGAG | ACAAGTCTGTTCTACACAAGTACA |
| | (SEQ ID NO: 1) | | (SEQ ID NO: 140) |
| DengueFw | TCGTCGGCAGCGTCAGATGTGTATAAGAGACAG | CTCGATAA | GGTTAGAGGAGACCCCTCC (SEQ |
| | (SEQ ID NO: 1) | | ID NO: 141) |
| CMVFw | TCGTCGGCAGCGTCAGATGTGTATAAGAGACAG | TTGTTCTC | CGAGTTCCCGGCGATGA (SEQ ID |
| | (SEQ ID NO: 1) | | NO: 142) |
| EBVFw | TCGTCGGCAGCGTCAGATGTGTATAAGAGACAG | TGACATCT | ACAATGTCGTCTTACACCATTGAG |
| | (SEQ ID NO: 1) | | (SEQ ID NO: 143) |
| RnasePFv | TCGTCGGCAGCGTCAGATGTGTATAAGAGACAG | TTCTGTTC | AGATTTGGACCTGCGAGCG (SEQ |
| | (SEQ ID NO: 1) | | ID NO: 122) |

OligoB-In (second primer) has the following elements:
From 5′ to 3′ direction 5′(CommonAdapter-CA2)-(PIDS2)-(3′TargetSpecificSequence-TSS2), where the CommonAdapter-CA2 can be the 3′ portion of the Illumina P7 Forked Adapter sequence that enables sequencing of the PIDS2 and SIDS2 regions flanking it on either side.
The OligoB-In constructs are as follows:

TABLE 19
Exemplar OligoB-In (second primer) sequence

| | CommonAdapter-CA2 | | |
|---|---|---|---|
| Name (Target) | (Illumina P7 Forked Adapter) (34 nt) | PIDS2 | 3′TargetSpecificSequence-TSS1 |
| ChikungunyaRv | GTGACTGGAGTTCAGACGTGTGCTCTTCCGATCT (SEQ | TCAGCACC | CTCCCGTGATCTTCTGCAC (SEQ ID |
| | ID NO: 144) | | NO: 145) |
| DengueRv | GTGACTGGAGTTCAGACGTGTGCTCTTCCGATCT (SEQ | GTTATCAC | TCCCAGCGTCAATATGCTG (SEQ ID |
| | ID NO: 144) | | NO: 146) |
| CMVRv | GTGACTGGAGTTCAGACGTGTGCTCTTCCGATCT (SEQ | ATGCGAAG | CCACCGCACTGAGGAATGTC (SEQ ID |
| | ID NO: 144) | | NO: 147) |
| EBVRv | GTGACTGGAGTTCAGACGTGTGCTCTTCCGATCT (SEQ | ACGTGTCC | ACAGACAATGGACTCCCTTAGTGG (SEQ |
| | ID NO: 144) | | ID NO: 148) |
| RNasePRv | GTGACTGGAGTTCAGACGTGTGCTCTTCCGATCT (SEQ | TGGCTCCT | GAGCGGCTGTCTCCACAAGT (SEQ ID |
| | ID NO: 144) | | NO: 149) |

### 2) Creation of Oligonucleotide Mix:

Custom synthesized oligos are ordered from custom oligosynthesizers such as IDT from the sequences listed above. Lyophilized oligonucleotides are reconstituted to a final concentration of 100 uM (micromolar) using Tris-EDTA Buffer (10 mM Tris pH 8.0, 1 mM EDTA). The total concentration of the OligoA-In pool in the reaction mix is 200 nM and the total concentration of the OligoB-In pool is 200 nM.

### 3) Amplification by Polymerase Chain Reaction:

5 uL of extracted viral nucleic acid was used as the starting template for a PCR using homebrew or standard commercially available reagents capable of reverse transcription and PCR in a single tube.

### 4) Incorporation of Sample Barcodes (SIDS) by PCR:

Sequences that enable tethering of constructs to the flow cell of the and barcoding of individual samples are incorporated through PCR with unique custom synthesized oligonucleotides. A unique pair of oligonucleotides—SOA and SOB—are used per sample. The oligonucleotides have the following structures:

SOA (third PCR primer)
The SOA is of the format:
(SequencingInstrumentSpecificTetheringAdapter1-TA1)-(SIDS1)-(CommonAdapter1-CA1), where the TA1 can be the Illumina P5 Binding Adapter and where CommonAdapter1 may be the 5′ portion of the Illumina Nextera P5 Sequencing Primer

TABLE 20
Exemplar SOA (third PCR primer) sequence

| Name | TA1 (25 nt) | SIDS1 | CA1 (14 nt) |
|---|---|---|---|
| SOA-N | AATGATACGGCGACCA | XXXXXXXXXXXX | TCGTCGGCAGCGTC |
| | CCGAGATCTACAC | (SEQ ID NO: | (SEQ ID NO: |
| | (SEQ ID NO: 63) | 64) | 65) |

where XXXXXXXXXXXX (SEQ ID NO:64) is a molecular barcode with lengths between 8 and 12 (can be more or less depending on the multiplexing required). A list of example barcodes is in appendix A. Criteria for selection of these barcodes from the pool of possible barcodes are:
Inter-barcode edit distance: The barcode pool is chosen such that each barcode in the pool is separated by an edit distance (Levenshtein) of 2 or more from any other barcode in the pool.
Hairpin structure evaluation: each barcode is evaluated for possible hairpin structures and only those where hairpin structures do not exist or have a melting temperature less than 0° C. are selected.
Interaction with SOB: Each complete SOA construct is evaluated for possible hybridization with each SOB structure. Only construct pairs which display no significant hybridization structures at 60 C are selected.
   SOB (fourth PCR primer)
   The SOB is of the format:
   (SequencingInstrumentSpecificTetheringAdapter2-TA2)-(SIDS2)-(CommonAdapter2-CA2), where TA2 can be the Illumina P7 Binding Adapter and where CA2 can be the 5′ portion of the Illumina P7 forked adapter sequencing primer.

TABLE 21
Exemplar SOB (fourth PCR primer) sequence

| Name | TA2 (24 nt) | SIDS2 | CA2 (19 nt) |
|---|---|---|---|
| SOB-M | CAAGCAGAAGACGG | YYYYYYYYYYYY | GTGACTGGAGTTC |
| | CATACGAGAT | (SEQ ID NO: | AGACGT (SEQ |
| | (SEQ ID NO: 66) | 67) | ID NO: 68) |

where YYYYYYYYYYYY (SEQ ID NO:67) is a molecular barcode with lengths between 8 and 12 (can be more or less depending on the multiplexing required). A list of example barcodes is in appendix B. Criteria for selection of these barcodes are similar to those set out for SOA—the same pool of barcodes can be used for both.

Unique pairs of oligonucleotides (one species of SOA and one species of SOB) at a final concentration of 400 nM are combined with the samples for the PCR. Commercial PCR master mixes such as Kapa HiFi Hotstart or Qiagen Quantitect Master Mix are used. The cycling conditions are initial denaturation at 95 C for 15 min, followed by 30 cycles of 95 C for 30 sec, 68 C for 45 sec and 72 C for 1 min 30 sec.

### 5) Pooling of Samples and Clean-Up:

The PCR products are quantified by a fluorometric assay (e.g. Thermo Qubit) and pooled at equimolar concentrations. The pool is purified using AMPure XP SPRI (solid phase reversible immobilization) technology. Alternative purification approaches which will be obvious to practitioners skilled in the art such as gel-based concentration, centrifugal spin column concentration, alcohol-salt precipitation, exonuclease and alkaline phosphatase treatment, etc. may also be used in the concentration/clean-up steps.

6) Quantification of Pool by Fluorometry and qPCR:

The purified library is quantified using fluorometric quantification method and molarity is corrected using qPCR with quantification standards.

7) Library Preparation and Loading of NGS: The Library is Prepared and Loaded onto the NGS According to Standard Illumina Protocol.

### 8) Probe Identification and Counting by NGS (Illumina SBS Chemistry)

The prepared library is sequenced in an Illumina sequencer by methods readily apparent to anyone skilled in the art.
a) Sequencer configuration: The PCR products are captured on the flow cell by the P5 and P7 tethering sequences at the ends of the construct. Each captured PCR product is clonally amplified to a cluster on the flow cell using the bridge PCR. Sequencing is initiated from the P5 end with the cluster tethered to the flow cell from the P7 end. Halfway through the cycle the molecule is flipped over and sequencing resumes from the P7 end with the cluster being anchored from the P5 end.
i) Read 1: The Illumina sequencer will read the amplicons generated from the ligated probes starting from the PIDS1 barcode. The sequencer is configured to read only the length of PIDS1 barcode (e.g. if the PIDS1 barcode is 12 bases long, there will be a 12 cycle Read 1)
ii) Indexing Read 1: Indexing cycles: The Illumina sequencer will read the sample specific barcode SIDS1 in the SOA region (XXXXXXXXXXXX)(SEQ ID NO:64) and
iii) Indexing Read 2: The barcode SIDS2 in the SOB region (YYYYYYYYYYYY) (SEQ ID NO:67) as part of its “Indexing cycles”. The sequencer reads only the number of bases specified in the barcode.
iv) Read 2: The Illumina sequencer will read the amplicons generated from the ligated probes starting from the PIDS2 barcode. The sequencer is configured to read only the length of PIDS2 barcode (e.g. if the PIDS2 barcode is 12 bases long, there will be 12 cycle Read 2)

This sequencer configuration of 12 (Read 1)+12 (Indexing Read 1)+12 (Indexing Read 2)+12 (Read 2)=48 total cycles allows for rapid sequencing; an Illumina NextSeq sequencer will complete this protocol in less than 6 hours. Further, the configuration of SIDS1/SIDS2 allows for multiplexing of large number of samples. On an Illumina NextSeq sequencer with a cluster capacity of 400 Million, more than 6000 samples can be multiplexed with an average of 4000 reads for each of the 15 constructs (6000 samples*15 constructs/sample*4000 reads/construct=400 Million reads)

### b) Demultiplexing Sample Specific Read Sequences:

The Illumina bcl2fastq software is configured with a SampleSheet.csv specifying the SIDS1/SIDS2 barcodes and upon execution, it demultiplexes reads corresponding to each unique pair of SIDS1/SIDS2.

### c) Quality Filter:

Each read is filtered such that the quality score for all bases in Read1/Read2 used to identify the read is above 30 on the phred scale (i.e. probability of base read being wrong is 1 in 1000).

### d) Assigning and Counting Reads Corresponding to Probes:

A custom software program is setup with a trie of all TSP barcodes (PIDS1 and PIDS2). In addition, all barcodes (derived by artificially inserting/deleting/substituting bases) within an edit distance of 2 from these barcodes are inserted into the trie as well. The leaf nodes of this trie structure stores information on the corresponding TSP.

For each sample:
i) For each read, the software walks the trie with the Read1/Read2 sequence. If both Read1 and Read2 are present in the trie and correspond to the same TSP sequence, the count for that TSP sequence for the sample is incremented.

Once constructed, the trie is read-only and can be shared across multiple threads/processors to rapidly process millions of reads. On an Intel i5-2310M CPU@ 2.5 GHz processor with four cores, 5 million reads can be processed in 1 minute. The 400 million reads from a NextSeq run can be processed within 1.5 hrs. With a more capable processor (more cores, higher CPU frequency), this can be sped up further (to less than 30 minutes).

### e) Calculating for the Presence of Pathogen Nucleic Acid Targets:

The raw NGS reads for the pathogens and reference target are counted. If raw reads for a particular pathogen or multiple pathogens and the reference target are above an empirically determined threshold value, the sample is interpreted as “positive” for that pathogen(s).

### Example 7: Testing for Chronic Myeloid Leukemia (CML), Acute Myeloid Leukemia (AML) and Acute Lymphoid Leukemia (ALL) Using the PCR Based Embodiment of the Present Invention

### 1) Identifying Target Specific Sequences (TSSs) to Create Target Specific Oligonucleotides (TSPs):

Targets of interest (fusion transcripts): BCR-ABL1 t(9,22) major (p210), BCR-ABL1 t(9,22) minor (p190), BCR-ABL1 t(9,22) micro (p230), PML-RARA t(15,17), CBFB-MYH11 inv(16), AML1-ETO t(8, 21), E2A-PBX2 t(1,19), TEL-AML1 t(12,21), MLL-AF4 t(4,11).
Reference Controls: GUS, B2M and ABL1 transcripts.
For each target and reference control, a first primer and a second primer (a sense primer, called OligoA-In and an antisense primer, called OligoB-In, respectively) are designed targeting a unique region within the relevant genome.
OligoA-In (first primer) has the following elements:
From 5′ to 3′ direction (CommonAdapter-CA1)-(PIDS1)-(5′TargetSpecificSequence-TSS1), where the CommonAdapter (CA1) can be the 3′ portion of the Illumina P5 Nextera Adapter sequence that enables sequencing of the PIDS1 and SIDS1 regions flanking it on either side, and
The OligoA-In constructs are as follows:

TABLE 22
Exemplar OligoA-In (first primer) sequence

| Name | CommonAdapter CA1 (Illumina | | |
|---|---|---|---|
| (Target) | Nextera P5) (27 nt) | PIDS1 | 5′TargetSpecificSequence-TSS1 |
| BCR-ABL1 | TCGTCGGCAGCGTCAGATGTGTATAAGAGACAG | TGGTGTTC | TCCGCTGACCATCAAYAAGGA (SEQ ID NO: 130) |
| major | (SEQ ID NO: 1) | | |
| BCR-ABL1 | TCGTCGGCAGCGTCAGATGTGTATAAGAGACAG | TGATTGAG | CTGGCCCAACGATGGCGA (SEQ ID NO: 150) |
| minor | (SEQ ID NO: 1) | | |
| BCR-ABL1 | TCGTCGGCAGCGTCAGATGTGTATAAGAGACAG | CTGAAGCG | GGAGGAGGTGGGCATCTACCG (SEQ ID NO: 151) |
| micro | (SEQ ID NO: 1) | | |
| PML-RARA 1 | TCGTCGGCAGCGTCAGATGTGTATAAGAGACAG | AGCTTCAT | TCTTCCTGCCCAACAGCAA (SEQ ID NO: 152) |
| | (SEQ ID NO: 1) | | |
| PML-RARA 2 | TCGTCGGCAGCGTCAGATGTGTATAAGAGACAG | ACTGCAGA | ACCTGGATGGACCGCCTAG (SEQ ID NO: 153) |
| | (SEQ ID NO: 1) | | |
| PML-RARA 3 | TCGTCGGCAGCGTCAGATGTGTATAAGAGACAG | CATGATGA | CCGATGGCTTCGACGAGTT (SEQ ID NO: 154) |
| | (SEQ ID NO: 1) | | |
| CBFB-MYH11 1 | TCGTCGGCAGCGTCAGATGTGTATAAGAGACAG | CTCCGGAC | CATTAGCACAACAGGCCTTTGA (SEQ ID NO: 155) |
| | (SEQ ID NO: 1) | | |
| AML1-ETO | TCGTCGGCAGCGTCAGATGTGTATAAGAGACAG | CATATATC | CACCTACCACAGAGCCATCAAA (SEQ ID NO: 156) |
| | (SEQ ID NO: 1) | | |
| E2A-PBX2 | TCGTCGGCAGCGTCAGATGTGTATAAGAGACAG | GTTAGTTC | CCAGCCTCATGCACAACCA (SEQ ID NO: 157) |
| | (SEQ ID NO: 1) | | |
| TEL-AML1 | TCGTCGGCAGCGTCAGATGTGTATAAGAGACAG | GTCATGAG | CTCTGTCTCCCCGCCTGAA (SEQ ID NO: 158) |
| | (SEQ ID NO: 1) | | |
| MLL-AF4 1 | TCGTCGGCAGCGTCAGATGTGTATAAGAGACAG | TCAGAGCG | CCCAAGTATCCCTGTAAAACAAAAA (SEQ ID |
| | (SEQ ID NO: 1) | | NO: 159) |
| MLL-AF4 2 | TCGTCGGCAGCGTCAGATGTGTATAAGAGACAG | GATCTCAT | GATGGAGTCCACAGGATCAGAGT (SEQ ID NO: 160) |
| | (SEQ ID NO: 1) | | |
| GUS | TCGTCGGCAGCGTCAGATGTGTATAAGAGACAG | GTATGCGA | GAAAATATGTGGTTGGAGAGCTCATT (SEQ ID |
| | (SEQ ID NO: 1) | | NO: 131) |
| B2M | TCGTCGGCAGCGTCAGATGTGTATAAGAGACAG | CTAATTAC | GAGTATGCCTGCCGTGTG (SEQ ID NO: 133) |
| | (SEQ ID NO: 1) | | |
| ABL1 | TCGTCGGCAGCGTCAGATGTGTATAAGAGACAG | CTGAGATA | TGGAGATAACACTCTAAGCATAACTAAAGGT (SEQ ID |
| | (SEQ ID NO: 1) | | NO: 135) |

OligoB-In (second primer) has the following elements:
From 5′ to 3′ direction 5′(CommonAdapter-CA2)-(PIDS2)-(3′TargetSpecificSequence-TSS2), where the CommonAdapter-CA2 can be the 3′ portion of the Illumina P7 Forked Adapter sequence that enables sequencing of the PIDS2 and SIDS2 regions flanking it on either side.

TABLE 23
Exemplar OligoB-In (second primer) sequence

| Name | CommonAdapter-CA2 (Illumina P7 Forked | | |
|---|---|---|---|
| (Target) | Adapter) (34 nt) | PIDS2 | 3′TargetSpecificSequence-TSS2 |
| BCR-ABL1 | GTGACTGGAGTTCAGACGTGTGCTCTTCCGATCT (SEQ | CCATGTTG | CACTCAGACCCTGAGGCTCAA (SEQ ID |
| | ID NO: 144) | | NO: 161) |
| PML-RARA | GTGACTGGAGTTCAGACGTGTGCTCTTCCGATCT (SEQ | ACACCGGC | GCTTGTAGATGCGGGGTAGAG (SEQ ID |
| | ID NO: 144) | | NO: 162) |
| CBFB-MYI-111 | GTGACTGGAGTTCAGACGTGTGCTCTTCCGATCT (SEQ | GTATCCTA | AGGGCCCGCTTGGACTT (SEQ ID NO: 163) |
| 1 | ID NO: 144) | | |
| CBFB-MYI-111 | GTGACTGGAGTTCAGACGTGTGCTCTTCCGATCT (SEQ | GGATGAGC | CCTCGTTAAGCATCCCTGTGA (SEQ ID |
| 2 | ID NO: 144) | | NO: 164) |
| CBFB-MYI-111 | GTGACTGGAGTTCAGACGTGTGCTCTTCCGATCT (SEQ | GAGACTAG | CTCTTTCTCCAGCGTCTGCTTAT (SEQ ID |
| 3 | ID NO: 144) | | NO: 165) |
| AML1-ETO | GTGACTGGAGTTCAGACGTGTGCTCTTCCGATCT (SEQ | GGCCTCTA | ATCCACAGGTGAGTCTGGCATT (SEQ ID |
| | ID NO: 144) | | NO: 166) |
| E2A-PBX2 | GTGACTGGAGTTCAGACGTGTGCTCTTCCGATCT (SEQ | CAATGATA | GGGCTCCTCGGATACTCAAAA (SEQ ID |
| | ID NO: 144) | | NO: 167) |
| TEL-AML1 | GTGACTGGAGTTCAGACGTGTGCTCTTCCGATCT (SEQ | CCTATCCA | CGGCTCGTGCTGGCAT |
| | ID NO: 144) | | (SEQ ID NO: 168) |
| MLL-AF4 | GTGACTGGAGTTCAGACGTGTGCTCTTCCGATCT (SEQ | TACAGACA | GAAAGGAAACTTGGATGGCTCA (SEQ ID |
| | ID NO: 144) | | NO: 169) |
| GUS | GTGACTGGAGTTCAGACGTGTGCTCTTCCGATCT (SEQ | CAGTACCA | CCGAGTGAAGATCCCCTTTTTA (SEQ ID |
| | ID NO: 144) | | NO: 170) |
| B2M | GTGACTGGAGTTCAGACGTGTGCTCTTCCGATCT (SEQ | AATTGAAT | AATCCAAATGCGGCATCT (SEQ ID NO: 171) |
| | ID NO: 144) | | |
| ABL1 | GTGACTGGAGTTCAGACGTGTGCTCTTCCGATCT (SEQ | AAGGTATC | GATGTAGTTGCTTGGGACCCA (SEQ ID |
| | ID NO: 144) | | NO: 172) |

### 2) Creation of Assay Specific Oligonucleotide Pools:

Custom synthesized oligos are ordered from custom oligonucleotide synthesis providers such as IDT from the sequences listed above. Lyophilized oligonucleotides are reconstituted to a final concentration of 100 uM (micromolar) using Tris-EDTA Buffer (10 mM Tris pH 8.0, 1 mM EDTA) and further diluted to 10 uM (micromolar) using Tris-EDTA Buffer.
The final concentration of each oligo in the 1× pool is 26 nanomolar.

### 3) Reverse Transcription Polymerase Chain Reaction for Amplification of Fusion Transcript:

RNA is reverse transcribed to cDNA using methods that are known to individuals skilled in the art, such as random priming, priming with oligodT primers and priming with target specific primers. 2 uL of cDNA is used as template for the amplification of fusion transcript in a master-mix containing Tris-HCl, KCl, (NH4)2SO4, 4 mM MgCl2, dNTPs, dUTP, HotStarTaq, Platinum taq polymerase and Uracil N-glycocylase (UNG). The cycling conditions are initial incubation at 37° C. for 10 min, initial denaturation at 95° C. for 15 min, followed by 45 cycles of denaturation 95° C. for 15 sec and annealing-extension at 64° C. for 45 sec.

### 4) Incorporation of Sample Barcodes (SIDS) by PCR:

Sequences that enable tethering of constructs to the flow cell of the and barcoding of individual samples are incorporated through PCR with unique custom synthesized oligonucleotides. A unique pair of oligonucleotides—SOA and SOB—are used per sample. The oligonucleotides have the following structures:
SOA (first PCR primer)
The SOA is of the format:
(SequencingInstrumentSpecificTetheringAdapter1-TA1)-(SIDS1)-(CommonAdapter1-CA1), where the TA1 can be the Illumina P5 Binding Adapter and where CommonAdapter1 may be the 5′ portion of the Illumina Nextera P5 Sequencing Primer

TABLE 24
Exemplar SOA (first PCR primer) sequence

| Name | TA1 (25 nt) | SIDS1 | CA1 (14 nt) |
|---|---|---|---|
| SOA-N | AATGATACGGCGACC | XXXXXXXXXXXX | TCGTCGGCAGCGTC |
| | ACCGAGATCTACAC | (SEQ ID NO: | (SEQ ID NO: 65) |
| | (SEQ ID NO: 63) | 64) | |

where XXXXXXXXXXXX (SEQ ID NO:64) is a molecular barcode with lengths between 8 and 12 (can be more or less depending on the multiplexing required). A list of example barcodes is in appendix A. Criteria for selection of these barcodes from the pool of possible barcodes are:
Inter-barcode edit distance: The barcode pool is chosen such that each barcode in the pool is separated by an edit distance (Levenshtein) of 2 or more from any other barcode in the pool.
Hairpin structure evaluation: each barcode is evaluated for possible hairpin structures and only those where hairpin structures do not exist or have a melting temperature less than 0° C. are selected.
Interaction with SOB: Each complete SOA construct is evaluated for possible hybridization with each SOB structure. Only construct pairs which display no significant hybridization structures at 60 C are selected.

### SOB

The SOB is of the format:
(SequencingInstrumentSpecificTetheringAdapter2-TA2)-(SIDS2)-(CommonAdapter2-CA2), where TA2 can be the Illumina P7 Binding Adapter and where CA2 can be the 5′ portion of the Illumina P7 forked adapter sequencing primer.

TABLE 25
Exemplar SOB (second PCR primer) sequence

| Name | TA2 (24 nt) | SIDS2 | CA2 (19 nt) |
|---|---|---|---|
| SOB-M | CAAGCAGAAGAC | YYYYYYYYYYYY | GTGACTGGAG |
| | GGCATACGAGAT | (SEQ ID NO: | TTCAGACGT |
| | (SEQ ID NO: | 67) | (SEQ ID NO: |
| | 66) | | 68) |

where YYYYYYYYYYYY (SEQ ID NO:67) is a molecular barcode with lengths between 8 and 12 (can be more or less depending on the multiplexing required). A list of example barcodes is in appendix B. Criteria for selection of these barcodes are similar to those set out for SOA—the same pool of barcodes can be used for both.

Unique pairs of oligonucleotides (one species of SOA and one species of SOB) at a final concentration of 400 nM are combined with the samples for the PCR. Commercial PCR master mixes such as Kapa HiFi Hotstart or Qiagen Quantitect Master Mix are used. The cycling conditions are initial denaturation at 95 C for 15 min, followed by 30 cycles of 95 C for 30 sec, 68 C for 45 sec and 72 C for 1 min 30 sec.

### 5) Pooling of Samples and Clean-Up:

The PCR products are quantified by a fluorometric assay (e.g. Thermo Qubit) and pooled at equimolar concentrations. The pool is purified using AMPure XP SPRI (solid phase reversible immobilization) technology. Alternative purification approaches which will be obvious to practitioners skilled in the art such as gel-based concentration, centrifugal spin column concentration, alcohol-salt precipitation, exonuclease and alkaline phosphatase treatment, etc. may also be used in the concentration/clean-up steps.

6) Quantification of Pool by Fluorometry and qPCR:

The purified library is quantified using fluorometric quantification method and molarity is corrected using qPCR with quantification standards.

7) Library Preparation and Loading of NGS: The Library is Prepared and Loaded onto the NGS According to Standard Illumina Protocol.

### 8) Probe Identification and Counting by NGS (Illumina SBS Chemistry)

The prepared library is sequenced in an Illumina sequencer by methods readily apparent to anyone skilled in the art.
a) Sequencer configuration: The PCR products are captured on the flow cell by the P5 and P7 tethering sequences at the ends of the construct. Each captured PCR product is clonally amplified to a cluster on the flow cell using the bridge PCR. Sequencing is initiated from the P5 end with the cluster tethered to the flow cell from the P7 end. Halfway through the cycle the molecule is flipped over and sequencing resumes from the P7 end with the cluster being anchored from the P5 end.
i) Read 1: The Illumina sequencer will read the amplicons generated from the ligated probes starting from the PIDS1 barcode. The sequencer is configured to read only the length of PIDS1 barcode (e.g. if the PIDS1 barcode is 12 bases long, there will be a 12 cycle Read 1)
ii) Indexing Read 1: Indexing cycles: The Illumina sequencer will read the sample specific barcode SIDS1 in the SOA region (XXXXXXXXXXXX)(SEQ ID NO:64) and
iii) Indexing Read 2: The barcode SIDS2 in the SOB region (YYYYYYYYYYYY) (SEQ ID NO:67) as part of its “Indexing cycles”. The sequencer reads only the number of bases specified in the barcode.
iv) Read 2: The Illumina sequencer will read the amplicons generated from the ligated probes starting from the PIDS2 barcode. The sequencer is configured to read only the length of PIDS2 barcode (e.g. if the PIDS2 barcode is 12 bases long, there will be 12 cycle Read 2)

This sequencer configuration of 12 (Read 1)+12 (Indexing Read 1)+12 (Indexing Read 2)+12 (Read 2)=48 total cycles allows for rapid sequencing; an Illumina NextSeq sequencer will complete this protocol in less than 6 hours. Further, the configuration of SIDS1/SIDS2 allows for multiplexing of large number of samples. On an Illumina NextSeq sequencer with a cluster capacity of 400 Million, more than 6000 samples can be multiplexed with an average of 4000 reads for each of the 15 constructs (6000 samples*15 constructs/sample*4000 reads/construct=400 Million reads).

### b) Demultiplexing Sample Specific Read Sequences:

The Illumina bcl2fastq software is configured with a SampleSheet.csv specifying the SIDS1/SIDS2 barcodes and upon execution, it demultiplexes reads corresponding to each unique pair of SIDS1/SIDS2.

### c) Quality Filter:

Each read is filtered such that the quality score for all bases in Read1/Read2 used to identify the read is above 30 on the phred scale (i.e. probability of base read being wrong is 1 in 1000).

### d) Assigning and Counting Reads Corresponding to Probes:

A custom software program is setup with a trie of all TSP barcodes (PIDS1 and PIDS2). In addition, all barcodes (derived by artificially inserting/deleting/substituting bases) within an edit distance of 2 from these barcodes are inserted into the trie as well. The leaf nodes of this trie structure stores information on the corresponding TSP.

For each sample:
i) For each read, the software walks the trie with the Read1/Read2 sequence. If both Read1 and Read2 are present in the trie and correspond to the same TSP sequence, the count for that TSP sequence for the sample is incremented.

Once constructed, the trie is read-only and can be shared across multiple threads/processors to rapidly process millions of reads. On an Intel i5-2310M CPU@ 2.5 GHz processor with four cores, 5 million reads can be processed in 1 minute. The 400 million reads from a NextSeq run can be processed within 1.5 hrs. With a more capable processor (more cores, higher CPU frequency), this can be sped up further (to less than 30 minutes).

### e) Calculating for the Presence of Fusion Transcripts:

The raw NGS reads for GUS, B2M and ABL1 reference transcripts as well as the fusion transcripts are counted. If raw reads for a particular fusion transcript is above a predetermined threshold value and the GUS, B2M and ABL1 counts are above empirically determined reference thresholds, the sample is interpreted as “positive” for that particular fusion transcript. The relative quantitation is calculated as the ratio of raw NGS reads for the particular fusion transcript and GUS, B2M and ABL1 transcripts.

### Example 8: Testing for Spinal Muscular Atrophy (SMA) Using the PCR Based Embodiment of the Present Invention

### 1) Identifying Target Specific Sequences (TSSs) to Create Target Specific Oligonucleotides (TSPs):

Targets of interest: SMN1 Exon 7 and SMN2 Exon 8

### Reference Controls: RNaseP, TERT and CFTR

For each target and reference control, a first primer and a second primer (a sense primer, called OligoA-In and an antisense primer, called OligoB-In, respectively) are designed targeting a unique region within the relevant genome.
OligoA-In (first primer) has the following elements:
From 5′ to 3′ direction (CommonAdapter-CA1)-(PIDS1)-(5′TargetSpecificSequence-TSS1), where the CommonAdapter (CA1) can be the 3′ portion of the Illumina P5 Nextera Adapter sequence that enables sequencing of the PIDS1 and SIDS1 regions flanking it on either side, and
The OligoA-In constructs are as follows:

TABLE 26
Exemplar OligoA-In (first primer) sequence

| Name | CommonAdapter CA1 (Illumina | | |
|---|---|---|---|
| (Target) | Nextera P5) (27 nt) | PIDS1 | 5′TargetSpecificSequence-TSS1 |
| SMN common | TCGTCGGCAGCGTCAGATGTGTATAAGAGACAG | GATCCTGC | CTTCCTTTATTTTCCTTACAGGGTT (SEQ ID NO: 3) |
| | (SEQ ID NO: 1) | | |
| TERT | TCGTCGGCAGCGTCAGATGTGTATAAGAGACAG | CACGCGTC | GGCACACGTGGCTTTTCG (SEQ ID NO: 118) |
| | (SEQ ID NO: 1) | | |
| CFTR | TCGTCGGCAGCGTCAGATGTGTATAAGAGACAG | CATGGCAG | AGCCGACACTTTGCTTGCTATG (SEQ ID NO: 120) |
| | (SEQ ID NO: 1) | | |
| RNaseP | TCGTCGGCAGCGTCAGATGTGTATAAGAGACAG | CTTCTCCG | AGATTTGGACCTGCGAGCG (SEQ ID NO: 122) |
| | (SEQ ID NO: 1) | | |

OligoB-In (second primer) has the following elements:
From 5′ to 3′ direction 5′(CommonAdapter-CA2)-(PIDS2)-(3′TargetSpecificSequence-TSS2), where the CommonAdapter-CA2 can be the 3′ portion of the Illumina P7 Forked Adapter sequence that enables sequencing of the PIDS2 and SIDS2 regions flanking it on either side.

TABLE 27
Exemplar OligoB-In (second primer) sequence

| Name | CommonAdapter-CA2 (Illumina P7 Forked | | |
|---|---|---|---|
| (Target) | Adapter) (34 nt) | PIDS2 | 3′TargetSpecificSequence-TSS2 |
| SMN1 Exon 7 | GTGACTGGAGTTCAGACGTGTGCTCTTCCGATCT (SEQ | GAAGGAAT | CCTTCCTTCTTTTTGATTTTGTCAG (SEQ ID |
| | ID NO: 144) | | NO: 114) |
| SMN2 Exon 7 | GTGACTGGAGTTCAGACGTGTGCTCTTCCGATCT (SEQ | CAGCCAGA | CCTTCCTTCTTTTTGATTTTGTCAA (SEQ ID |
| | ID NO: 144) | | NO: 116) |
| TERT | GTGACTGGAGTTCAGACGTGTGCTCTTCCGATCT (SEQ | GTGCATGA | GGTGAACCTCGTAAGTTTATGCAA (SEQ ID |
| | ID NO: 144) | | NO: 173) |
| CFTR | GTGACTGGAGTTCAGACGTGTGCTCTTCCGATCT (SEQ | TCTTGGAC | GACATAGGTGCTTGAAGAACAGAATG (SEQ ID |
| | ID NO: 144) | | NO: 174) |
| RNaseP | GTGACTGGAGTTCAGACGTGTGCTCTTCCGATCT (SEQ | GTGTTATC | GAGCGGCTGTCTCCACAAGT (SEQ ID NO: 149) |
| | ID NO: 144) | | |

### 2) Creation of Assay Specific Oligonucleotide Pools:

Custom synthesized oligos are ordered from custom oligonucleotide synthesis providers such as IDT from the sequences listed above. Lyophilized oligonucleotides are reconstituted to a final concentration of 100 uM (micromolar) using Tris-EDTA Buffer (10 mM Tris pH 8.0, 1 mM EDTA) and further diluted to 10 uM (micromolar) using Tris-EDTA Buffer. The final concentration of each oligo in the 1× pool is 300 nanomolar.

### 3) Polymerase Chain Reaction for Amplification of the Targets:

2 uL of DNA is used as template for the amplification of the targets in a mastermix containing Tris-HCl, KCl, (NH4)2SO4, 4 mM MgCl2, dNTPs, dUTP, HotStarTaq, Platinum taq polymerase. The cycling conditions are initial denaturation at 95° C. for 15 min, followed by 35 cycles of denaturation 95° C. for 20 sec, annealing at 63° C. for 30 sec and extension at 72° C. for 15 sec.

### 4) Incorporation of Sample Barcodes (SIDS) by PCR:

Sequences that enable tethering of constructs to the flow cell of the and barcoding of individual samples are incorporated through PCR with unique custom synthesized oligonucleotides. A unique pair of oligonucleotides—SOA and SOB—are used per sample. The oligonucleotides have the following structures:
SOA (first PCR primer)
The SOA is of the format:
(SequencingInstrumentSpecificTetheringAdapter1-TA1)-(SIDS1)-(CommonAdapter1-CA1), where the TA1 can be the Illumina P5 Binding Adapter and where CommonAdapter1 may be the 5′ portion of the Illumina Nextera P5 Sequencing Primer

TABLE 27
Exemplar SOA (first PCR primer) sequence

| Name | TA1 (25 nt) | SIDS1 | CA1 (14 nt) |
|---|---|---|---|
| SOA-N | AATGATACGGCGACC | XXXXXXXXXXXX | TCGTCGGCAGCGTC |
| | ACCGAGATCTACAC | (SEQ ID NO: | (SEQ ID NO: |
| | (SEQ ID NO: 63) | 64) | 65) |

where XXXXXXXXXXXX (SEQ ID NO:64) is a molecular barcode with lengths between 8 and 12 (can be more or less depending on the multiplexing required). A list of example barcodes is in appendix A. Criteria for selection of these barcodes from the pool of possible barcodes are:
Inter-barcode edit distance: The barcode pool is chosen such that each barcode in the pool is separated by an edit distance (Levenshtein) of 2 or more from any other barcode in the pool.
Hairpin structure evaluation: each barcode is evaluated for possible hairpin structures and only those where hairpin structures do not exist or have a melting temperature less than 0° C. are selected.
Interaction with SOB: Each complete SOA construct is evaluated for possible hybridization with each SOB structure. Only construct pairs which display no significant hybridization structures at 60 C are selected.

### SOB

The SOB is of the format:
(SequencingInstrumentSpecificTetheringAdapter2-TA2)-(SIDS2)-(CommonAdapter2-CA2), where TA2 can be the Illumina P7 Binding Adapter and where CA2 can be the 5′ portion of the Illumina P7 forked adapter sequencing primer.

TABLE 28
Exemplar SOB (second PCR primer) sequence

| Name | TA2 (24 nt) | SIDS2 | CA2 (19 nt) |
|---|---|---|---|
| SOB-M | CAAGCAGAAGACG | YYYYYYYYYYYY | GTGACTGGAGT |
| | GCATACGAGAT | (SEQ ID NO: | TCAGACGT |
| | (SEQ ID NO: 66) | 67) | (SEQ ID NO: |
| | | | 68) |

where YYYYYYYYYYYY (SEQ ID NO:67) is a molecular barcode with lengths between 8 and 12 (can be more or less depending on the multiplexing required). A list of example barcodes is in appendix B. Criteria for selection of these barcodes are similar to those set out for SOA—the same pool of barcodes can be used for both.

Unique pairs of oligonucleotides (one species of SOA and one species of SOB) at a final concentration of 400 nM are combined with the samples for the PCR. Commercial PCR master mixes such as Kapa HiFi Hotstart or Qiagen Quantitect Master Mix are used. The cycling conditions are initial denaturation at 95 C for 15 min, followed by 30 cycles of 95 C for 30 sec, 68 C for 45 sec and 72 C for 1 min 30 sec.

### 5) Pooling of Samples and Clean-Up:

The PCR products are quantified by a fluorometric assay (e.g. Thermo Qubit) and pooled at equimolar concentrations. The pool is purified using AMPure XP SPRI (solid phase reversible immobilization) technology. Alternative purification approaches which will be obvious to practitioners skilled in the art such as gel-based concentration, centrifugal spin column concentration, alcohol-salt precipitation, exonuclease and alkaline phosphatase treatment, etc. may also be used in the concentration/clean-up steps.

6) Quantification of Pool by Fluorometry and qPCR:

The purified library is quantified using fluorometric quantification method and molarity is corrected using qPCR with quantification standards.

7) Library Preparation and Loading of NGS: The Library is Prepared and Loaded onto the NGS According to Standard Illumina Protocol.

### 8) Probe Identification and Counting by NGS (Illumina SBS Chemistry)

The prepared library is sequenced in an Illumina sequencer by methods readily apparent to anyone skilled in the art.
a) Sequencer configuration: The PCR products are captured on the flow cell by the P5 and P7 tethering sequences at the ends of the construct. Each captured PCR product is clonally amplified to a cluster on the flow cell using the bridge PCR. Sequencing is initiated from the P5 end with the cluster tethered to the flow cell from the P7 end. Halfway through the cycle the molecule is flipped over and sequencing resumes from the P7 end with the cluster being anchored from the P5 end.
i) Read 1: The Illumina sequencer will read the amplicons generated from the ligated probes starting from the PIDS1 barcode. The sequencer is configured to read only the length of PIDS1 barcode (e.g. if the PIDS1 barcode is 12 bases long, there will be a 12 cycle Read 1)
ii) Indexing Read 1: Indexing cycles: The Illumina sequencer will read the sample specific barcode SIDS1 in the SOA region (XXXXXXXXXXXX)(SEQ ID NO:64) and
iii) Indexing Read 2: The barcode SIDS2 in the SOB region (YYYYYYYYYYYY) (SEQ ID NO:67) as part of its “Indexing cycles”. The sequencer reads only the number of bases specified in the barcode.
iv) Read 2: The Illumina sequencer will read the amplicons generated from the ligated probes starting from the PIDS2 barcode. The sequencer is configured to read only the length of PIDS2 barcode (e.g. if the PIDS2 barcode is 12 bases long, there will be 12 cycle Read 2)

This sequencer configuration of 12 (Read 1)+12 (Indexing Read 1)+12 (Indexing Read 2)+12 (Read 2)=48 total cycles allows for rapid sequencing; an Illumina NextSeq sequencer will complete this protocol in less than 6 hours. Further, the configuration of SIDS1/SIDS2 allows for multiplexing of large number of samples. On an Illumina NextSeq sequencer with a cluster capacity of 400 Million, more than 6000 samples can be multiplexed with an average of 4000 reads for each of the 15 constructs (6000 samples*15 constructs/sample*4000 reads/construct=400 Million reads).

### b) Demultiplexing Sample Specific Read Sequences:

The Illumina bcl2fastq software is configured with a SampleSheet.csv specifying the SIDS1/SIDS2 barcodes and upon execution, it demultiplexes reads corresponding to each unique pair of SIDS1/SIDS2.

### c) Quality Filter:

Each read is filtered such that the quality score for all bases in Read1/Read2 used to identify the read is above 30 on the phred scale (i.e. probability of base read being wrong is 1 in 1000).

### d) Assigning and Counting Reads Corresponding to Probes:

A custom software program is setup with a trie of all TSP barcodes (PIDS1 and PIDS2). In addition, all barcodes (derived by artificially inserting/deleting/substituting bases) within an edit distance of 2 from these barcodes are inserted into the trie as well. The leaf nodes of this trie structure stores information on the corresponding TSP.

For each sample:
i) For each read, the software walks the trie with the Read1/Read2 sequence. If both Read1 and Read2 are present in the trie and correspond to the same TSP sequence, the count for that TSP sequence for the sample is incremented.

Once constructed, the trie is read-only and can be shared across multiple threads/processors to rapidly process millions of reads. On an Intel i5-2310M CPU@ 2.5 GHz processor with four cores, 5 million reads can be processed in 1 minute. The 400 million reads from a NextSeq run can be processed within 1.5 hrs. With a more capable processor (more cores, higher CPU frequency), this can be sped up further (to less than 30 minutes).

### (e) Calculating Copy Numbers:

Copy numbers are calculated by intra-sample normalization, Averaging per-TSP in Control Samples and Inter-sample normalization:
(e1) Intra-sample normalization by total number of reads: For each sample, the read counts for each construct are normalized by the total number of reads for the sample yielding a number from 0.0 to 1.0.
(e2) Averaging per-TSP in Control Samples: Across all control samples, the normalized values for each TSP from step (e1) are weighted by the number of known copies in the control sample and averaged.
(e3) Inter-sample normalization for each sample: For each TSP, the normalized value from step (e1) is divided by the per-TSP average normalized value from step (e2) to yield the ratio/copy number.

### 9) Interpretation and Reporting:

The ratios from the normalization algorithm are used to categorize the samples:
i) a value between 0.8-1.2 is interpreted as normal diploid, whereas
ii) a value >0.3 and <0.80 is interpreted as a heterozygous deletion and
iii) a value >1.3 and <1.75 is interpreted as a heterozygous duplication; a value >1.75 is interpreted as >3 copies.
iv) a value <0.1 is interpreted as a homozygous deletion.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

### APPENDICES

APPENDIX A
Exemplar SOA (first PCR primer) constructs:

| Sequence | SEQ ID NO |
|---|---|
| AATGATACGGCGACCACCGAGATCTACAC-AACAACAACACC-TCGTCGGCAGCGTC | SEQ ID NO: 175 |
| AATGATACGGCGACCACCGAGATCTACAC-AACAAGTCCTCG-TCGTCGGCAGCGTC | SEQ ID NO: 176 |
| AATGATACGGCGACCACCGAGATCTACAC-AACACCGATTAG-TCGTCGGCAGCGTC | SEQ ID NO: 177 |
| AATGATACGGCGACCACCGAGATCTACAC-AACAGGAGCGCA-TCGTCGGCAGCGTC | SEQ ID NO: 178 |
| AATGATACGGCGACCACCGAGATCTACAC-AACCGAGAACTT-TCGTCGGCAGCGTC | SEQ ID NO: 179 |
| AATGATACGGCGACCACCGAGATCTACAC-AACCTGCCACAT-TCGTCGGCAGCGTC | SEQ ID NO: 180 |
| AATGATACGGCGACCACCGAGATCTACAC-AACTAATTGCGG-TCGTCGGCAGCGTC | SEQ ID NO: 181 |
| AATGATACGGCGACCACCGAGATCTACAC-AACTTCTCTTCC-TCGTCGGCAGCGTC | SEQ ID NO: 182 |
| AATGATACGGCGACCACCGAGATCTACAC-AAGAACCGAGCC-TCGTCGGCAGCGTC | SEQ ID NO: 183 |
| AATGATACGGCGACCACCGAGATCTACAC-AAGAAGGTTACG-TCGTCGGCAGCGTC | SEQ ID NO: 184 |
| AATGATACGGCGACCACCGAGATCTACAC-AAGACTATGACC-TCGTCGGCAGCGTC | SEQ ID NO: 185 |
| AATGATACGGCGACCACCGAGATCTACAC-AAGAGTGGAAGT-TCGTCGGCAGCGTC | SEQ ID NO: 186 |
| AATGATACGGCGACCACCGAGATCTACAC-AAGCGGTCATTA-TCGTCGGCAGCGTC | SEQ ID NO: 187 |
| AATGATACGGCGACCACCGAGATCTACAC-AAGGTCCGGTTG-TCGTCGGCAGCGTC | SEQ ID NO: 188 |
| AATGATACGGCGACCACCGAGATCTACAC-AATAGCAATCGG-TCGTCGGCAGCGTC | SEQ ID NO: 189 |
| AATGATACGGCGACCACCGAGATCTACAC-AATGCTTACTGG-TCGTCGGCAGCGTC | SEQ ID NO: 190 |
| AATGATACGGCGACCACCGAGATCTACAC-AATTACGAGAGG-TCGTCGGCAGCGTC | SEQ ID NO: 191 |
| AATGATACGGCGACCACCGAGATCTACAC-ACAACCTTCAGC-TCGTCGGCAGCGTC | SEQ ID NO: 192 |
| AATGATACGGCGACCACCGAGATCTACAC-ACAATGACAAGG-TCGTCGGCAGCGTC | SEQ ID NO: 193 |
| AATGATACGGCGACCACCGAGATCTACAC-ACAGGTAATAGG-TCGTCGGCAGCGTC | SEQ ID NO: 194 |
| AATGATACGGCGACCACCGAGATCTACAC-ACATTAACCTCG-TCGTCGGCAGCGTC | SEQ ID NO: 195 |
| AATGATACGGCGACCACCGAGATCTACAC-ACCGAACGCCAT-TCGTCGGCAGCGTC | SEQ ID NO: 196 |
| AATGATACGGCGACCACCGAGATCTACAC-ACCGTCAGAGTA-TCGTCGGCAGCGTC | SEQ ID NO: 197 |
| AATGATACGGCGACCACCGAGATCTACAC-ACGCCATACATA-TCGTCGGCAGCGTC | SEQ ID NO: 198 |
| AATGATACGGCGACCACCGAGATCTACAC-ACGCTGAAGAAT-TCGTCGGCAGCGTC | SEQ ID NO: 199 |
| AATGATACGGCGACCACCGAGATCTACAC-ACGGTTCTAATC-TCGTCGGCAGCGTC | SEQ ID NO: 200 |
| AATGATACGGCGACCACCGAGATCTACAC-ACTATCGCACTT-TCGTCGGCAGCGTC | SEQ ID NO: 201 |
| AATGATACGGCGACCACCGAGATCTACAC-AGAGCATAAGGA-TCGTCGGCAGCGTC | SEQ ID NO: 202 |
| AATGATACGGCGACCACCGAGATCTACAC-AGATTCCGCCGT-TCGTCGGCAGCGTC | SEQ ID NO: 203 |
| AATGATACGGCGACCACCGAGATCTACAC-AGGAAGAGAGAG-TCGTCGGCAGCGTC | SEQ ID NO: 204 |
| AATGATACGGCGACCACCGAGATCTACAC-AGTGTGGTTCTC-TCGTCGGCAGCGTC | SEQ ID NO: 205 |
| AATGATACGGCGACCACCGAGATCTACAC-ATAAGACTCACC-TCGTCGGCAGCGTC | SEQ ID NO: 206 |
| AATGATACGGCGACCACCGAGATCTACAC-ATCGTCGTGCCT-TCGTCGGCAGCGTC | SEQ ID NO: 207 |
| AATGATACGGCGACCACCGAGATCTACAC-ATGGAGATTGGT-TCGTCGGCAGCGTC | SEQ ID NO: 208 |
| AATGATACGGCGACCACCGAGATCTACAC-ATTCATACCAGC-TCGTCGGCAGCGTC | SEQ ID NO: 209 |
| AATGATACGGCGACCACCGAGATCTACAC-CAATACGCTGCA-TCGTCGGCAGCGTC | SEQ ID NO: 210 |
| AATGATACGGCGACCACCGAGATCTACAC-CACCTAACTATC-TCGTCGGCAGCGTC | SEQ ID NO: 211 |
| AATGATACGGCGACCACCGAGATCTACAC-CAGAGCAACCAT-TCGTCGGCAGCGTC | SEQ ID NO: 212 |
| AATGATACGGCGACCACCGAGATCTACAC-CAGCTCGCCTTA-TCGTCGGCAGCGTC | SEQ ID NO: 213 |
| AATGATACGGCGACCACCGAGATCTACAC-CATAATCAGTCC-TCGTCGGCAGCGTC | SEQ ID NO: 214 |
| AATGATACGGCGACCACCGAGATCTACAC-CATCAAGAACGG-TCGTCGGCAGCGTC | SEQ ID NO: 215 |
| AATGATACGGCGACCACCGAGATCTACAC-CATCTAGGTTGT-TCGTCGGCAGCGTC | SEQ ID NO: 216 |
| AATGATACGGCGACCACCGAGATCTACAC-CATGTGCTATTC-TCGTCGGCAGCGTC | SEQ ID NO: 217 |
| AATGATACGGCGACCACCGAGATCTACAC-CATGTGGAGGAA-TCGTCGGCAGCGTC | SEQ ID NO: 218 |
| AATGATACGGCGACCACCGAGATCTACAC-CCAATTCTACCG-TCGTCGGCAGCGTC | SEQ ID NO: 219 |
| AATGATACGGCGACCACCGAGATCTACAC-CCACACCACATA-TCGTCGGCAGCGTC | SEQ ID NO: 220 |
| AATGATACGGCGACCACCGAGATCTACAC-CCACCGCTTCTT-TCGTCGGCAGCGTC | SEQ ID NO: 221 |
| AATGATACGGCGACCACCGAGATCTACAC-CCATCTTAATCG-TCGTCGGCAGCGTC | SEQ ID NO: 222 |
| AATGATACGGCGACCACCGAGATCTACAC-CCGAATAGAACT-TCGTCGGCAGCGTC | SEQ ID NO: 223 |
| AATGATACGGCGACCACCGAGATCTACAC-CCGCAGTCCTAT-TCGTCGGCAGCGTC | SEQ ID NO: 224 |
| AATGATACGGCGACCACCGAGATCTACAC-CCGGTGAGTTAA-TCGTCGGCAGCGTC | SEQ ID NO: 225 |
| AATGATACGGCGACCACCGAGATCTACAC-CGTAAGTGATGG-TCGTCGGCAGCGTC | SEQ ID NO: 226 |
| AATGATACGGCGACCACCGAGATCTACAC-CTAACCATGAAG-TCGTCGGCAGCGTC | SEQ ID NO: 227 |
| AATGATACGGCGACCACCGAGATCTACAC-CTAGTGTTCAAG-TCGTCGGCAGCGTC | SEQ ID NO: 228 |
| AATGATACGGCGACCACCGAGATCTACAC-CTCCGATCCAAT-TCGTCGGCAGCGTC | SEQ ID NO: 229 |
| AATGATACGGCGACCACCGAGATCTACAC-CTGAACTCCGCA-TCGTCGGCAGCGTC | SEQ ID NO: 230 |
| AATGATACGGCGACCACCGAGATCTACAC-CTTACATGCCTC-TCGTCGGCAGCGTC | SEQ ID NO: 231 |
| AATGATACGGCGACCACCGAGATCTACAC-GAAGTCTCCATT-TCGTCGGCAGCGTC | SEQ ID NO: 232 |
| AATGATACGGCGACCACCGAGATCTACAC-GAGCCTTAGTCT-TCGTCGGCAGCGTC | SEQ ID NO: 233 |
| AATGATACGGCGACCACCGAGATCTACAC-GAGGAGGTGTTG-TCGTCGGCAGCGTC | SEQ ID NO: 234 |
| AATGATACGGCGACCACCGAGATCTACAC-GATAACCGCATA-TCGTCGGCAGCGTC | SEQ ID NO: 235 |
| AATGATACGGCGACCACCGAGATCTACAC-GATGGACTGAGG-TCGTCGGCAGCGTC | SEQ ID NO: 236 |
| AATGATACGGCGACCACCGAGATCTACAC-GCAGCACCGTAA-TCGTCGGCAGCGTC | SEQ ID NO: 237 |
| AATGATACGGCGACCACCGAGATCTACAC-GCCTATAATTCC-TCGTCGGCAGCGTC | SEQ ID NO: 238 |
| AATGATACGGCGACCACCGAGATCTACAC-GCTACTCACCAA-TCGTCGGCAGCGTC | SEQ ID NO: 239 |
| AATGATACGGCGACCACCGAGATCTACAC-GCTGCAATATAC-TCGTCGGCAGCGTC | SEQ ID NO: 240 |
| AATGATACGGCGACCACCGAGATCTACAC-GGTAGATCATTG-TCGTCGGCAGCGTC | SEQ ID NO: 241 |
| AATGATACGGCGACCACCGAGATCTACAC-GTACTGTTCCTT-TCGTCGGCAGCGTC | SEQ ID NO: 242 |
| AATGATACGGCGACCACCGAGATCTACAC-GTCTCCGTCTCT-TCGTCGGCAGCGTC | SEQ ID NO: 243 |
| AATGATACGGCGACCACCGAGATCTACAC-GTGTTATGTTGG-TCGTCGGCAGCGTC | SEQ ID NO: 244 |
| AATGATACGGCGACCACCGAGATCTACAC-GTTCTCATAGCT-TCGTCGGCAGCGTC | SEQ ID NO: 245 |
| AATGATACGGCGACCACCGAGATCTACAC-TAGCCACGTTCC-TCGTCGGCAGCGTC | SEQ ID NO: 246 |
| AATGATACGGCGACCACCGAGATCTACAC-TAGCTTAACACC-TCGTCGGCAGCGTC | SEQ ID NO: 247 |
| AATGATACGGCGACCACCGAGATCTACAC-TAGTGACGATGG-TCGTCGGCAGCGTC | SEQ ID NO: 248 |
| AATGATACGGCGACCACCGAGATCTACAC-TATAGAGCAAGG-TCGTCGGCAGCGTC | SEQ ID NO: 249 |
| AATGATACGGCGACCACCGAGATCTACAC-TATCATTCGCTC-TCGTCGGCAGCGTC | SEQ ID NO: 250 |
| AATGATACGGCGACCACCGAGATCTACAC-TCCGTATTAGCC-TCGTCGGCAGCGTC | SEQ ID NO: 251 |
| AATGATACGGCGACCACCGAGATCTACAC-TGAGAGCCTATT-TCGTCGGCAGCGTC | SEQ ID NO: 252 |
| AATGATACGGCGACCACCGAGATCTACAC-TGGTTGGAGTAA-TCGTCGGCAGCGTC | SEQ ID NO: 253 |
| AATGATACGGCGACCACCGAGATCTACAC-TGTTGCTTGATC-TCGTCGGCAGCGTC | SEQ ID NO: 254 |
| AATGATACGGCGACCACCGAGATCTACAC-TTAACGGTCGAG-TCGTCGGCAGCGTC | SEQ ID NO: 255 |
| AATGATACGGCGACCACCGAGATCTACAC-TTACCAACCGAA-TCGTCGGCAGCGTC | SEQ ID NO: 256 |
| AATGATACGGCGACCACCGAGATCTACAC-TTATGTGCTGCG-TCGTCGGCAGCGTC | SEQ ID NO: 257 |
| AATGATACGGCGACCACCGAGATCTACAC-TTCCTCACCTCC-TCGTCGGCAGCGTC | SEQ ID NO: 258 |
| AATGATACGGCGACCACCGAGATCTACAC-TTGGAAGTACGG-TCGTCGGCAGCGTC | SEQ ID NO: 259 |

APPENDIX B
Exemplar SOB(second PCR primer) constructs:

| Sequence | SEQ ID NO |
|---|---|
| CAAGCAGAAGACGGCATACGAGAT-AACAACAACACC-GTGACTGGAGTTCAGACGT | SEQ ID NO: 260 |
| CAAGCAGAAGACGGCATACGAGAT-AACAAGTCCTCG-GTGACTGGAGTTCAGACGT | SEQ ID NO: 261 |
| CAAGCAGAAGACGGCATACGAGAT-AACACCGATTAG-GTGACTGGAGTTCAGACGT | SEQ ID NO: 262 |
| CAAGCAGAAGACGGCATACGAGAT-AACAGGAGCGCA-GTGACTGGAGTTCAGACGT | SEQ ID NO: 263 |
| CAAGCAGAAGACGGCATACGAGAT-AACCGAGAACTT-GTGACTGGAGTTCAGACGT | SEQ ID NO: 264 |
| CAAGCAGAAGACGGCATACGAGAT-AACCTGCCACAT-GTGACTGGAGTTCAGACGT | SEQ ID NO: 265 |
| CAAGCAGAAGACGGCATACGAGAT-AACTAATTGCGG-GTGACTGGAGTTCAGACGT | SEQ ID NO: 266 |
| CAAGCAGAAGACGGCATACGAGAT-AACTTCTCTTCC-GTGACTGGAGTTCAGACGT | SEQ ID NO: 267 |
| CAAGCAGAAGACGGCATACGAGAT-AAGAACCGAGCC-GTGACTGGAGTTCAGACGT | SEQ ID NO: 268 |
| CAAGCAGAAGACGGCATACGAGAT-AAGAAGGTTACG-GTGACTGGAGTTCAGACGT | SEQ ID NO: 269 |
| CAAGCAGAAGACGGCATACGAGAT-AAGACTATGACC-GTGACTGGAGTTCAGACGT | SEQ ID NO: 270 |
| CAAGCAGAAGACGGCATACGAGAT-AAGAGTGGAAGT-GTGACTGGAGTTCAGACGT | SEQ ID NO: 271 |
| CAAGCAGAAGACGGCATACGAGAT-AAGCGGTCATTA-GTGACTGGAGTTCAGACGT | SEQ ID NO: 272 |
| CAAGCAGAAGACGGCATACGAGAT-AAGGTCCGGTTG-GTGACTGGAGTTCAGACGT | SEQ ID NO: 273 |
| CAAGCAGAAGACGGCATACGAGAT-AATAGCAATCGG-GTGACTGGAGTTCAGACGT | SEQ ID NO: 274 |
| CAAGCAGAAGACGGCATACGAGAT-AATGCTTACTGG-GTGACTGGAGTTCAGACGT | SEQ ID NO: 275 |
| CAAGCAGAAGACGGCATACGAGAT-AATTACGAGAGG-GTGACTGGAGTTCAGACGT | SEQ ID NO: 276 |
| CAAGCAGAAGACGGCATACGAGAT-ACAACCTTCAGC-GTGACTGGAGTTCAGACGT | SEQ ID NO: 277 |
| CAAGCAGAAGACGGCATACGAGAT-ACAATGACAAGG-GTGACTGGAGTTCAGACGT | SEQ ID NO: 278 |
| CAAGCAGAAGACGGCATACGAGAT-ACAGGTAATAGG-GTGACTGGAGTTCAGACGT | SEQ ID NO: 279 |
| CAAGCAGAAGACGGCATACGAGAT-ACATTAACCTCG-GTGACTGGAGTTCAGACGT | SEQ ID NO: 280 |
| CAAGCAGAAGACGGCATACGAGAT-ACCGAACGCCAT-GTGACTGGAGTTCAGACGT | SEQ ID NO: 281 |
| CAAGCAGAAGACGGCATACGAGAT-ACCGTCAGAGTA-GTGACTGGAGTTCAGACGT | SEQ ID NO: 282 |
| CAAGCAGAAGACGGCATACGAGAT-ACGCCATACATA-GTGACTGGAGTTCAGACGT | SEQ ID NO: 283 |
| CAAGCAGAAGACGGCATACGAGAT-ACGCTGAAGAAT-GTGACTGGAGTTCAGACGT | SEQ ID NO: 284 |
| CAAGCAGAAGACGGCATACGAGAT-ACGGTTCTAATC-GTGACTGGAGTTCAGACGT | SEQ ID NO: 285 |
| CAAGCAGAAGACGGCATACGAGAT-ACTATCGCACTT-GTGACTGGAGTTCAGACGT | SEQ ID NO: 286 |
| CAAGCAGAAGACGGCATACGAGAT-AGAGCATAAGGA-GTGACTGGAGTTCAGACGT | SEQ ID NO: 287 |
| CAAGCAGAAGACGGCATACGAGAT-AGATTCCGCCGT-GTGACTGGAGTTCAGACGT | SEQ ID NO: 288 |
| CAAGCAGAAGACGGCATACGAGAT-AGGAAGAGAGAG-GTGACTGGAGTTCAGACGT | SEQ ID NO: 289 |
| CAAGCAGAAGACGGCATACGAGAT-AGTGTGGTTCTC-GTGACTGGAGTTCAGACGT | SEQ ID NO: 290 |
| CAAGCAGAAGACGGCATACGAGAT-ATAAGACTCACC-GTGACTGGAGTTCAGACGT | SEQ ID NO: 291 |
| CAAGCAGAAGACGGCATACGAGAT-ATCGTCGTGCCT-GTGACTGGAGTTCAGACGT | SEQ ID NO: 292 |
| CAAGCAGAAGACGGCATACGAGAT-ATGGAGATTGGT-GTGACTGGAGTTCAGACGT | SEQ ID NO: 293 |
| CAAGCAGAAGACGGCATACGAGAT-ATTCATACCAGC-GTGACTGGAGTTCAGACGT | SEQ ID NO: 294 |
| CAAGCAGAAGACGGCATACGAGAT-CAATACGCTGCA-GTGACTGGAGTTCAGACGT | SEQ ID NO: 295 |
| CAAGCAGAAGACGGCATACGAGAT-CACCTAACTATC-GTGACTGGAGTTCAGACGT | SEQ ID NO: 296 |
| CAAGCAGAAGACGGCATACGAGAT-CAGAGCAACCAT-GTGACTGGAGTTCAGACGT | SEQ ID NO: 297 |
| CAAGCAGAAGACGGCATACGAGAT-CAGCTCGCCTTA-GTGACTGGAGTTCAGACGT | SEQ ID NO: 298 |
| CAAGCAGAAGACGGCATACGAGAT-CATAATCAGTCC-GTGACTGGAGTTCAGACGT | SEQ ID NO: 299 |
| CAAGCAGAAGACGGCATACGAGAT-CATCAAGAACGG-GTGACTGGAGTTCAGACGT | SEQ ID NO: 300 |
| CAAGCAGAAGACGGCATACGAGAT-CATCTAGGTTGT-GTGACTGGAGTTCAGACGT | SEQ ID NO: 301 |
| CAAGCAGAAGACGGCATACGAGAT-CATGTGCTATTC-GTGACTGGAGTTCAGACGT | SEQ ID NO: 302 |
| CAAGCAGAAGACGGCATACGAGAT-CATGTGGAGGAA-GTGACTGGAGTTCAGACGT | SEQ ID NO: 303 |
| CAAGCAGAAGACGGCATACGAGAT-CCAATTCTACCG-GTGACTGGAGTTCAGACGT | SEQ ID NO: 304 |
| CAAGCAGAAGACGGCATACGAGAT-CCACACCACATA-GTGACTGGAGTTCAGACGT | SEQ ID NO: 305 |
| CAAGCAGAAGACGGCATACGAGAT-CCACCGCTTCTT-GTGACTGGAGTTCAGACGT | SEQ ID NO: 306 |
| CAAGCAGAAGACGGCATACGAGAT-CCATCTTAATCG-GTGACTGGAGTTCAGACGT | SEQ ID NO: 307 |
| CAAGCAGAAGACGGCATACGAGAT-CCGAATAGAACT-GTGACTGGAGTTCAGACGT | SEQ ID NO: 308 |
| CAAGCAGAAGACGGCATACGAGAT-CCGCAGTCCTAT-GTGACTGGAGTTCAGACGT | SEQ ID NO: 309 |
| CAAGCAGAAGACGGCATACGAGAT-CCGGTGAGTTAA-GTGACTGGAGTTCAGACGT | SEQ ID NO: 310 |
| CAAGCAGAAGACGGCATACGAGAT-CGTAAGTGATGG-GTGACTGGAGTTCAGACGT | SEQ ID NO: 311 |
| CAAGCAGAAGACGGCATACGAGAT-CTAACCATGAAG-GTGACTGGAGTTCAGACGT | SEQ ID NO: 312 |
| CAAGCAGAAGACGGCATACGAGAT-CTAGTGTTCAAG-GTGACTGGAGTTCAGACGT | SEQ ID NO: 313 |
| CAAGCAGAAGACGGCATACGAGAT-CTCCGATCCAAT-GTGACTGGAGTTCAGACGT | SEQ ID NO: 314 |
| CAAGCAGAAGACGGCATACGAGAT-CTGAACTCCGCA-GTGACTGGAGTTCAGACGT | SEQ ID NO: 315 |
| CAAGCAGAAGACGGCATACGAGAT-CTTACATGCCTC-GTGACTGGAGTTCAGACGT | SEQ ID NO: 316 |
| CAAGCAGAAGACGGCATACGAGAT-GAAGTCTCCATT-GTGACTGGAGTTCAGACGT | SEQ ID NO: 317 |
| CAAGCAGAAGACGGCATACGAGAT-GAGCCTTAGTCT-GTGACTGGAGTTCAGACGT | SEQ ID NO: 318 |
| CAAGCAGAAGACGGCATACGAGAT-GAGGAGGTGTTG-GTGACTGGAGTTCAGACGT | SEQ ID NO: 319 |
| CAAGCAGAAGACGGCATACGAGAT-GATAACCGCATA-GTGACTGGAGTTCAGACGT | SEQ ID NO: 320 |
| CAAGCAGAAGACGGCATACGAGAT-GATGGACTGAGG-GTGACTGGAGTTCAGACGT | SEQ ID NO: 321 |
| CAAGCAGAAGACGGCATACGAGAT-GCAGCACCGTAA-GTGACTGGAGTTCAGACGT | SEQ ID NO: 322 |
| CAAGCAGAAGACGGCATACGAGAT-GCCTATAATTCC-GTGACTGGAGTTCAGACGT | SEQ ID NO: 323 |
| CAAGCAGAAGACGGCATACGAGAT-GCTACTCACCAA-GTGACTGGAGTTCAGACGT | SEQ ID NO: 324 |
| CAAGCAGAAGACGGCATACGAGAT-GCTGCAATATAC-GTGACTGGAGTTCAGACGT | SEQ ID NO: 325 |
| CAAGCAGAAGACGGCATACGAGAT-GGTAGATCATTG-GTGACTGGAGTTCAGACGT | SEQ ID NO: 326 |
| CAAGCAGAAGACGGCATACGAGAT-GTACTGTTCCTT-GTGACTGGAGTTCAGACGT | SEQ ID NO: 327 |
| CAAGCAGAAGACGGCATACGAGAT-GTCTCCGTCTCT-GTGACTGGAGTTCAGACGT | SEQ ID NO: 328 |
| CAAGCAGAAGACGGCATACGAGAT-GTGTTATGTTGG-GTGACTGGAGTTCAGACGT | SEQ ID NO: 329 |
| CAAGCAGAAGACGGCATACGAGAT-GTTCTCATAGCT-GTGACTGGAGTTCAGACGT | SEQ ID NO: 330 |
| CAAGCAGAAGACGGCATACGAGAT-TAGCCACGTTCC-GTGACTGGAGTTCAGACGT | SEQ ID NO: 331 |
| CAAGCAGAAGACGGCATACGAGAT-TAGCTTAACACC-GTGACTGGAGTTCAGACGT | SEQ ID NO: 332 |
| CAAGCAGAAGACGGCATACGAGAT-TAGTGACGATGG-GTGACTGGAGTTCAGACGT | SEQ ID NO: 333 |
| CAAGCAGAAGACGGCATACGAGAT-TATAGAGCAAGG-GTGACTGGAGTTCAGACGT | SEQ ID NO: 334 |
| CAAGCAGAAGACGGCATACGAGAT-TATCATTCGCTC-GTGACTGGAGTTCAGACGT | SEQ ID NO: 335 |
| CAAGCAGAAGACGGCATACGAGAT-TCCGTATTAGCC-GTGACTGGAGTTCAGACGT | SEQ ID NO: 336 |
| CAAGCAGAAGACGGCATACGAGAT-TGAGAGCCTATT-GTGACTGGAGTTCAGACGT | SEQ ID NO: 337 |
| CAAGCAGAAGACGGCATACGAGAT-TGGTTGGAGTAA-GTGACTGGAGTTCAGACGT | SEQ ID NO: 338 |
| CAAGCAGAAGACGGCATACGAGAT-TGTTGCTTGATC-GTGACTGGAGTTCAGACGT | SEQ ID NO: 339 |
| CAAGCAGAAGACGGCATACGAGAT-TTAACGGTCGAG-GTGACTGGAGTTCAGACGT | SEQ ID NO: 340 |
| CAAGCAGAAGACGGCATACGAGAT-TTACCAACCGAA-GTGACTGGAGTTCAGACGT | SEQ ID NO: 341 |
| CAAGCAGAAGACGGCATACGAGAT-TTATGTGCTGCG-GTGACTGGAGTTCAGACGT | SEQ ID NO: 342 |
| CAAGCAGAAGACGGCATACGAGAT-TTCCTCACCTCC-GTGACTGGAGTTCAGACGT | SEQ ID NO: 343 |
| CAAGCAGAAGACGGCATACGAGAT-TTGGAAGTACGG-GTGACTGGAGTTCAGACGT | SEQ ID NO: 344 |

What is claimed is:

1. A method of determining the abundance of each of one or more target nucleotide sequences in each of one or more samples, the method comprising:
(a) generating nucleic acid constructs from the one or more target nucleotide sequences in the more or more samples, each of the nucleic acid constructs comprising:
(i) a probe-identification sequence (PIDS) that identifies the target nucleotide sequence from which the nucleic acid construct is derived; and
(ii) a sample identification sequence (SIDS) that identifies the sample from which the nucleic acid construct is derived;
(b) pooling the nucleic acid constructs from the one or more samples into a single combined sample;
(c) quantifying the PIDS and the SIDS of the nucleic acid constructs, thereby obtaining quantification results; and
(d) determining the abundance of each of the one or more target nucleotide sequences for each of the one or more samples based on the quantification results.

2. The method of claim 1, wherein the nucleic acid constructs are generated by:
(a) contacting each of the one or more samples with a first set of target-specific probes (TSP1s) and a second set of target-specific probes (TSP2s) under sufficient conditions and for a sufficient time to allow the TSP1s and TSP2s to hybridize to their target nucleotide sequences, wherein each of the TSP1s comprises, from the 5′ end to the 3′ end, a first common adaptor (CA1), a first PIDS (PIDS1) and a first target-specific sequence (TSS1), and wherein each of the TSP2s comprises, from the 5′ end to the 3′ end, a second target-specific sequence (TSS2), a second PIDS (PIDS2) and a second common adaptor (CA2);
(b) contacting each of the one or more samples containing TSP1s and TSP2 with a ligase under sufficient conditions and for a sufficient time, such that if the TSS1 and TSS2 hybridized to the target nucleotide sequence and the 3′ end of TSS1 and the 5′ end of TSS2 are immediately adjacent to each other, then the TSP1 and TSP2 are ligated by the ligase to form a ligation product (LP); and
(c) amplifying by PCR the LPs to produce the nucleic acid constructs, the PCR amplification step comprising:
(i) amplifying the LPs by PCR using a first PCR primer comprising, from the 5′ end to the 3′ end, a first tethering adaptor (TA1), a first SIDS (SIDS1), and a sequence corresponding to the CA1; and
(ii) amplifying the IAs by PCR using a second PCR primer comprising, from the 5′ end to the 3′ end, a second TA (TA2), a second SIDS (SIDS2), and a sequence corresponding to the CA2, thereby generating the nucleic acid construct.

3. The method of claim 1, wherein the nucleic acid constructs are generated by:
(a) contacting each of the one or more samples with a first set of target-specific probes (TSP1s) and a second set of target-specific probes (TSP2s) under sufficient conditions and for a sufficient time to allow the TSP1s and TSP2s to hybridize to their target nucleotide sequences, wherein each of the TSP1s comprises, from the 5′ end to the 3′ end, a first common adaptor (CA1), a first PIDS (PIDS1) and a first target-specific sequence (TSS1), and wherein each of the TSP2s comprises, from the 5′ end to the 3′ end, a second target-specific sequence (TSS2), a second PIDS (PIDS2) and a second common adaptor (CA2);
(b) contacting each of the one or more samples containing TSP1s and TSP2s with a polymerase and nucleic acids under sufficient condition and for a sufficient time to allow extension of a TSP1 at the 3′ end, if the TSP1 is hybridized to a target nucleotide sequence,
(c) contacting each of the one or more samples containing TSP1s and TSP2s with a ligase under sufficient condition and for a sufficient time to allow ligation of a TSP1 with a TSP2 if the 3′ end of the TSP1 is immediately adjacent to the 5′ end of the TSP2;
(d) amplifying by PCR the LPs to produce a one or more nucleic acid constructs, the PCR amplification step comprising:
(i) amplifying the LP by PCR using a first PCR primer comprising a TA1, the SIDS, and a sequence corresponding to the CA1, thereby generating a plurality of intermediate amplicons (IAs), each IAs comprising a TA1; and
(ii) amplifying the IAs by PCR using a second PCR primer comprising a TA2, a sample identification sequence (SIDS), and a sequence corresponding to the CA2, thereby generating the amplicons.

4. The method of claim 1, wherein the nucleic acid constructs are generated by:
(a) amplifying the target nucleotide sequences by PCR using a first primer, the first primer comprising, from the 5′ end to the 3′ end, a first common adaptor (CA1), a first PIDS (PIDS1), and a first TSS (TSS1), thereby generating first intermediary PCR products (IPP1);
(b) amplifying the IPP1 by PCR using a second primer, the second primer comprising, from the 5′ end to the 3′ end, a second common adaptor (CA2), a second PIDS (PIDS2), and a second TSS (TSS2), thereby generating second intermediary PCR products (IPP2);
(c) amplifying the IPP2 by PCR using a third primer, the third primer comprising, from the 5′ end to the 3′ end, a first Tethering Adapter (TA1), a first SIDS (SIDS1), and a sequence corresponding to CA1, thereby generating third intermediary PCR products (IPP3);
(d) amplifying the IPP3 by PCR using a fourth primer, the fourth primer comprising, from the 5′ end to the 3′ end, a second Tethering Adapter (TA2), a second SIDS (SIDS2), and a sequence corresponding to CA2, thereby generating the nucleic acid constructs.

5. The method of any one of claims 1-4, wherein the PIDSs can comprise distinct nucleotide sequences chosen from the nucleotide sequences disclosed in Appendix A or Appendix B.

6. The method of any one of claims 1-5, wherein the SIDS can comprise distinct nucleotide sequences chosen from the nucleotide sequences disclosed in Appendix A or Appendix B.

7. The method of any one of claims 1-6, wherein at least one of the one or more target nucleotide sequences is associated with a genetic disorder, a cancer, or an infectious disease.

8. A method of claim 7, wherein the genetic disorder is selected from spinal muscular atrophy, Duchenne muscular dystrophy, Becker muscular dystrophy, alpha thalassemia, microdeletion and microduplication syndromes associated with neurodevelopmental disorder, autism, atypical hemolytic uraemic syndrome, beta thalassemia, congenital adrenal hyperplasia, thrombophilia, lysosomal storage disorders, Prader-Willi syndrome, Angelmann syndrome. Beckwith-Wiedemann syndrome, Silver-Russell Syndrome, or fragile-X syndrome.

9. The method of claim 7, wherein the cancer is selected from hereditary breast cancer, hereditary ovarian cancer, prostate cancer, renal cancer, cerebellar cancer, colon cancer, or retinoblastoma.

10. A method of claim 7, wherein the infectious disease is caused by chikungunya virus, dengue virus, plasmodium, Zika, cytomegalovirus, Epstein-Barr virus, herpes simplex virus, varicella zoster virus, adenovirus, human immunodeficiency virus, hepatitis B virus, hepatitis C virus, human papillomavirus, Neisseria gonorrhoeae (NG), Chlamydia trachomatis (CT), Trichomonas vaginalis (TV), Mycoplasma sp., influenza virus, S. pneumoniae, K. pneumonia, S. aureus, Salmonella, fungus, Pseudomonas, E. coli, Proteus, Acinetobacter, influenza A virus subtype H1N1, or severe acute respiratory syndrome coronavirus 2 (SARS-CoV-2).

11. The method of any one of claims 1-10, wherein the nucleic acid constructs are double-stranded DNA.

12. The method of any one of claims 2-11, wherein the 5′ ends of the TSP2s are phosphorylated.

13. The method of any one of claims 1-12, wherein at least one of the target nucleotide sequences comprises a sequence corresponding to a genomic DNA sequence that contains an genetic aberration, the genetic aberration being a single nucleotide polymorphism, insertion, deletion, duplication, rearrangement, truncation, or translocation, as compared to a wild-type genomic DNA sequence.

14. The method of any one of claims 1-13, wherein at least one of the target nucleotide sequences comprises nucleotide sequences having abnormal methylation status as compared to a wild-type DNA sequence.

15. The method of any one of claims 1-14, wherein the one or more samples comprise samples from one or more subjects.

16. The method of any one of claims 1-15, wherein the one or more samples comprise blood, bone marrow, cerebrospinal fluid, pleural fluid, or urine.

17. The method of any one of claims 1-16, wherein the one or more samples are from a single subject, obtained at different times.

18. The method of any one of claims 1-11, wherein the one or more samples comprise at least 100 samples, at least 1,000 samples, at least 10,000 samples, at least 100,000 samples, at least 1,000,000 samples, at least 10,000,000 samples, at least 100,000,000 samples, or at least 1,000,000,000 samples.

19. The method of any one of claims 1-11, wherein the one or more target nucleotide sequences comprise at least 100 target nucleotide sequences, at least 1,000 target nucleotide sequences, at least 10,000 target nucleotide sequences, at least 100,000 target nucleotide sequences, at least 1,000,000 target nucleotide sequences, at least 10,000,000 target nucleotide sequences, at least 100,000,000 target nucleotide sequences, or at least 1,000,000,000 target nucleotide sequences.

20. The method of any one of claims 1-19, wherein the PIDSs and/or the SIDSs comprise oligonucleotides having specific sequences.

21. The method of claim 20, wherein the PIDS is between 4 and 7 nucleotides, between 8 and 12 nucleotides, between 13 and 16 nucleotides, between 17-20 nucleotides, or greater than 21 nucleotides in length.

22. The method of claim 20, wherein the SIDS is between 4 and 7 nucleotides, between 8 and 12 nucleotides, between 13 and 16 nucleotides, between 17-20 nucleotides, or greater than 21 nucleotides in length.

23. The method of any one of claims 1-22, wherein the PIDS and/or the SIDS comprises a Raman spectrometry tag or a mass spectrometry tag.

24. The method of any one of claims 1-22, wherein the PIDS and/or the SIDS comprises a fluorescent tag.

25. The method of claim 24, wherein the fluorescent tag comprises a quantum dot or a NanoString probe.

26. The method of any one of claims 1-25, wherein quantification of the PIDS and/or the SIDS measures relative abundance of PIDS and/or SIDS as compared to PIDS and/or SIDS associated with one or more reference TSSs (RTSSs).

27. The method of claim 26, wherein the RTSSs comprise OCA2, KLKB, IL4, SETX, PARD3, HIPK3, AMOT, LAMA42, SPAST, and/or PPHLNJ.

28. The method of any one of claims 1-27, wherein the PIDS1 and PIDS2 targeting the same target nucleotide sequence are different from each other.

29. The method of any one of claims 1-28, wherein the PIDS1 and PIDS2 targeting the same target nucleotide sequence are the same.

30. The method of any one of claims 1-29, wherein SIDS1 and SIDS2 targeting the same target nucleotide sequence are different from each other.

31. The method of any one of claims 1-30, wherein SIDS1 and SIDS2 targeting the same target nucleotide sequence are the same.

32. The method of any one of claims 1-31, wherein each of the PIDSs and/or SIDSs comprise sequences having an edit distance (Levenshtein) of 2 or more from any other PIDSs and/or SIDSs.

33. The method of any one of claims 2-32, wherein the TSS is between 10 and 50 nucleotides, between 15 and 40 nucleotides, or between 20 and 30 nucleotides in length.

34. The method of any one of claims 2-33, wherein the CA is between 10 and 60 nucleotides, between 20 and 50 nucleotides, or between 30 and 40 nucleotides in length.

35. The method of any one of claims 1-34, wherein the one or more target nucleotide sequences comprise one or more reference sequences.

36. The method of claim 2, wherein the TSS1 and the TSS2 each comprises a nucleic acid sequence that is complementary to at least a portion of the target nucleotide sequence.

37. The method of claim 1, wherein determining the abundance of each of the one or more target nucleotide sequences for each of the one or more samples comprises:
accessing the quantification results, each of the quantification results being associated with at least one read sequence;
classifying the quantification results, using a classifier engine comprising one or more processing devices, by identifying (i) one of the one or more target nucleotide sequences, and (ii) one of the one or more samples, from each of the corresponding read sequences.

38. The method of claim 37, wherein the at least one read sequence comprises a first read sequence usable for identifying the one of the one or more target nucleotide sequences, and a second read sequence usable for one of the one or more samples.

39. The method of claim 37, wherein the classifier engine implements a classification process based on a trie search structure.

40. The method of claim 37, comprising:
determining, by the classifier engine, that an edit distance between a particular read sequence and a particular target nucleotide sequence satisfies a threshold condition; and
responsive to determining that the edit distance between the particular read sequence and the particular target nucleotide sequence satisfies the threshold condition, identifying the particular read sequence as the particular target nucleotide sequence.

41. The method of claim 40, wherein the threshold condition is determined to be satisfied if the edit distance between the particular read sequence and the particular target nucleotide sequence is less than 3.

42. A kit for determining the abundance of each of a plurality of target sequences in each of a plurality of samples, the kit comprising:
(a) a set of TSP1s corresponding to the plurality of target sequences and reference sequences and reference sequences, the set of TSP1s each comprising, from the 5′ end to the 3′ end, a first common adaptor (CA1), a first PIDS (PIDS1) and a first target-specific sequence (TSS1);
(b) a set of TSP2s corresponding to the plurality of target sequences and reference sequences, the set of TSP1s each comprising, from the 5′ end to the 3′ end, a second target-specific sequence (TSS2), a second PIDS (PIDS2) and a second common adaptor (CA2);
(c) a set of first PCR primers comprising, from the 5′ end to the 3′ end, a first tethering adaptor (TA1), a first SIDS (SIDS1), and a sequence corresponding to the CA1;
(d) a set of second PCR primers comprising, from the 5′ end to the 3′ end, a second tethering adaptor (TA2), a second SIDS (SIDS2), and a sequence corresponding to the CA2; and
(e) optionally, a ligase and/or a polymerase.

43. A kit for determining the abundance of each of a plurality of target sequences having specific sequences in each of a plurality of samples, the kit comprising:
(a) a set of first primers corresponding to the plurality of target sequences and reference sequences, the set of first primers each comprising, from the 5′ end to the 3′ end, a first common adaptor (CA1), a first PIDS (PIDS1), and a first TSS (TSS1), thereby generating first intermediary PCR products (IPP1);
(b) a set of second primers corresponding to the plurality of target sequences and reference sequences, the set of second primers each comprising, from the 5′ end to the 3′ end, a second common adaptor (CA2), a second PIDS (PIDS2), and a second TSS (TSS2), thereby generating second intermediary PCR products (IPP2);
(c) a set of third primers corresponding to the sequences of the CA1, the set of second primers each comprising, from the 5′ end to the 3′ end, a first Tethering Adapter (TA1), a first SIDS (SIDS1), and a sequence corresponding to CA1;
(d) a set of fourth primers corresponding to the sequences of the CA2, the set of second primers each comprising, from the 5′ end to the 3′ end a second Tethering adapter (TA2), a second SIDS (SIDS2), and a sequence corresponding to CA2; and
(e) optionally, a polymerase.

44. A method of diagnosing one or more conditions in one or more subjects by detecting the presence or absence of one or more nucleic acid alteration in the plurality of subjects, the method comprising:
(a) obtaining a plurality of samples from the plurality of subjects;
(b) performing the method of any one of claims 1-41 to determine the abundance of each of the plurality of target genes in each of the plurality of samples; and
(c) diagnosing the one or more conditions that are each associated with the abundance of one or more of the plurality of target genes for each of the plurality of samples.

45. The method of claim 44, further comprising treating the subjects for the condition diagnosed.


SEQUENCE LISTING (344 sequences)

SEQ ID NO: 1 - DNA, 33 bp, Artificial Sequence
  Description of Artificial Sequence: Synthetic probe
tcgtcggcagcgtcagatgtgtataagagacag

SEQ ID NO: 2 - DNA, 8 bp, Artificial Sequence
  Description of Artificial Sequence: Synthetic probe
agttaaca

SEQ ID NO: 3 - DNA, 29 bp, Artificial Sequence
  Description of Artificial Sequence: Synthetic probe
aacttcctttattttccttacagggtttc

SEQ ID NO: 4 - DNA, 8 bp, Artificial Sequence
  Description of Artificial Sequence: Synthetic probe
tgcagcag

SEQ ID NO: 5 - DNA, 23 bp, Artificial Sequence
  Description of Artificial Sequence: Synthetic probe
gtgctggcctcccacccccaccc

SEQ ID NO: 6 - DNA, 8 bp, Artificial Sequence
  Description of Artificial Sequence: Synthetic probe
ctcttcta

SEQ ID NO: 7 - DNA, 30 bp, Artificial Sequence
  Description of Artificial Sequence: Synthetic probe
gagcaccttccttctttttgattttgtcta

SEQ ID NO: 8 - DNA, 8 bp, Artificial Sequence
  Description of Artificial Sequence: Synthetic probe
ctaggacg

SEQ ID NO: 9 - DNA, 35 bp, Artificial Sequence
  Description of Artificial Sequence: Synthetic probe
aacctttcaactttttaacatctgaactttttaac

SEQ ID NO: 10 - DNA, 8 bp, Artificial Sequence
  Description of Artificial Sequence: Synthetic probe
tgtacgtc

SEQ ID NO: 11 - DNA, 33 bp, Artificial Sequence
  Description of Artificial Sequence: Synthetic probe
ccaaatgcaatgtgaaatattttactggactct

SEQ ID NO: 12 - DNA, 8 bp, Artificial Sequence
  Description of Artificial Sequence: Synthetic probe
gaccaaca

SEQ ID NO: 13 - DNA, 33 bp, Artificial Sequence
  Description of Artificial Sequence: Synthetic probe
gctcaaccttgatccaagacaagtcctgattgc

SEQ ID NO: 14 - DNA, 8 bp, Artificial Sequence
  Description of Artificial Sequence: Synthetic probe
tgtggcga

SEQ ID NO: 15 - DNA, 28 bp, Artificial Sequence
  Description of Artificial Sequence: Synthetic probe
ccaaatgcccaatactgccagatgaggt

SEQ ID NO: 16 - DNA, 8 bp, Artificial Sequence
  Description of Artificial Sequence: Synthetic probe
tctctcta

SEQ ID NO: 17 - DNA, 33 bp, Artificial Sequence
  Description of Artificial Sequence: Synthetic probe
ggacacaagtgcgatatcaccttacaggagatc

SEQ ID NO: 18 - DNA, 8 bp, Artificial Sequence
  Description of Artificial Sequence: Synthetic probe
agaggagc

SEQ ID NO: 19 - DNA, 28 bp, Artificial Sequence
  Description of Artificial Sequence: Synthetic probe
tgcgtaatgggaaaactgagtgttacct

SEQ ID NO: 20 - DNA, 8 bp, Artificial Sequence
  Description of Artificial Sequence: Synthetic probe
actaactc

SEQ ID NO: 21 - DNA, 33 bp, Artificial Sequence
  Description of Artificial Sequence: Synthetic probe
gagagtctgtatccacagccagtgatcagcctt

SEQ ID NO: 22 - DNA, 8 bp, Artificial Sequence
  Description of Artificial Sequence: Synthetic probe
cacaatag

SEQ ID NO: 23 - DNA, 33 bp, Artificial Sequence
  Description of Artificial Sequence: Synthetic probe
gcatagttcaccaagtcccagtgggcttaaatc

SEQ ID NO: 24 - DNA, 8 bp, Artificial Sequence
  Description of Artificial Sequence: Synthetic probe
gattggca

SEQ ID NO: 25 - DNA, 28 bp, Artificial Sequence
  Description of Artificial Sequence: Synthetic probe
cagacgagaaccggaacttgaggcaaga

SEQ ID NO: 26 - DNA, 8 bp, Artificial Sequence
  Description of Artificial Sequence: Synthetic probe
caattgga

SEQ ID NO: 27 - DNA, 28 bp, Artificial Sequence
  Description of Artificial Sequence: Synthetic probe
gcaaattcggactcgatgccaagaatcc

SEQ ID NO: 28 - DNA, 8 bp, Artificial Sequence
  Description of Artificial Sequence: Synthetic probe
tcgaagta

SEQ ID NO: 29 - DNA, 33 bp, Artificial Sequence
  Description of Artificial Sequence: Synthetic probe
gtacagtctgctggagatgacagagtacttgta

SEQ ID NO: 30 - DNA, 8 bp, Artificial Sequence
  Description of Artificial Sequence: Synthetic probe
gacttcgc

SEQ ID NO: 31 - DNA, 28 bp, Artificial Sequence
  Description of Artificial Sequence: Synthetic probe
gaaaaggaacttgctgaggctgcaagca

SEQ ID NO: 32 - DNA, 43 bp, Artificial Sequence
  Description of Artificial Sequence: Synthetic probe
agacaaaatcaaaaagaaggaaggtgctcacattccttaaatt

SEQ ID NO: 33 - DNA, 8 bp, Artificial Sequence
  Description of Artificial Sequence: Synthetic probe
cttggcca

SEQ ID NO: 34 - DNA, 34 bp, Artificial Sequence
  Description of Artificial Sequence: Synthetic probe
agatcggaagagcacacgtctgaactccagtcac

SEQ ID NO: 35 - DNA, 24 bp, Artificial Sequence
  Description of Artificial Sequence: Synthetic probe
cagtcttttacagatggtttttca

SEQ ID NO: 36 - DNA, 8 bp, Artificial Sequence
  Description of Artificial Sequence: Synthetic probe
tctgatca

SEQ ID NO: 37 - DNA, 33 bp, Artificial Sequence
  Description of Artificial Sequence: Synthetic probe
aaaccctgtaaggaaaataaaggaagttaaaaa

SEQ ID NO: 38 - DNA, 8 bp, Artificial Sequence
  Description of Artificial Sequence: Synthetic probe
caggttct

SEQ ID NO: 39 - DNA, 41 bp, Artificial Sequence
  Description of Artificial Sequence: Synthetic probe
tgttcaaaaacatttgttttccacaaaccataaagttttac

SEQ ID NO: 40 - DNA, 8 bp, Artificial Sequence
  Description of Artificial Sequence: Synthetic probe
tcatatgt

SEQ ID NO: 41 - DNA, 25 bp, Artificial Sequence
  Description of Artificial Sequence: Synthetic probe
tttgaaaaaccatctgtaaaagact

SEQ ID NO: 42 - DNA, 8 bp, Artificial Sequence
  Description of Artificial Sequence: Synthetic probe
caccggtc

SEQ ID NO: 43 - DNA, 33 bp, Artificial Sequence
  Description of Artificial Sequence: Synthetic probe
agaagtgatcttcacaaacattggaggagctgc

SEQ ID NO: 44 - DNA, 8 bp, Artificial Sequence
  Description of Artificial Sequence: Synthetic probe
gttgcaag

SEQ ID NO: 45 - DNA, 28 bp, Artificial Sequence
  Description of Artificial Sequence: Synthetic probe
gcacattccacccaaggtgtttgctatt

SEQ ID NO: 46 - DNA, 8 bp, Artificial Sequence
  Description of Artificial Sequence: Synthetic probe
tgtgatag

SEQ ID NO: 47 - DNA, 33 bp, Artificial Sequence
  Description of Artificial Sequence: Synthetic probe
atcaaaactttgaacagcctcacagagcagaag

SEQ ID NO: 48 - DNA, 8 bp, Artificial Sequence
  Description of Artificial Sequence: Synthetic probe
gaggcctg

SEQ ID NO: 49 - DNA, 29 bp, Artificial Sequence
  Description of Artificial Sequence: Synthetic probe
ttccatccagactcaagagaactttccgg

SEQ ID NO: 50 - DNA, 8 bp, Artificial Sequence
  Description of Artificial Sequence: Synthetic probe
agaattca

SEQ ID NO: 51 - DNA, 33 bp, Artificial Sequence
  Description of Artificial Sequence: Synthetic probe
cccactctctggagagacaaatgaatggaaacc

SEQ ID NO: 52 - DNA, 8 bp, Artificial Sequence
  Description of Artificial Sequence: Synthetic probe
tggctgga

SEQ ID NO: 53 - DNA, 33 bp, Artificial Sequence
  Description of Artificial Sequence: Synthetic probe
cccgtctgttaccatccccaaccattcatcaga

SEQ ID NO: 54 - DNA, 8 bp, Artificial Sequence
  Description of Artificial Sequence: Synthetic probe
aggaaggc

SEQ ID NO: 55 - DNA, 28 bp, Artificial Sequence
  Description of Artificial Sequence: Synthetic probe
gttggaaggatgctatgagaaggtggca

SEQ ID NO: 56 - DNA, 8 bp, Artificial Sequence
  Description of Artificial Sequence: Synthetic probe
acatgcag

SEQ ID NO: 57 - DNA, 28 bp, Artificial Sequence
  Description of Artificial Sequence: Synthetic probe
acttggctgcagcagctgctattgcttc

SEQ ID NO: 58 - DNA, 8 bp, Artificial Sequence
  Description of Artificial Sequence: Synthetic probe
tcgagacg

SEQ ID NO: 59 - DNA, 33 bp, Artificial Sequence
  Description of Artificial Sequence: Synthetic probe
atgggtgcaactaataggccacaagagcttgat

SEQ ID NO: 60 - DNA, 8 bp, Artificial Sequence
  Description of Artificial Sequence: Synthetic probe
agttggtg

SEQ ID NO: 61 - DNA, 28 bp, Artificial Sequence
  Description of Artificial Sequence: Synthetic probe
agtgggctgctgaaaagctagagaaatc

SEQ ID NO: 62 - DNA, 8 bp, Artificial Sequence
  Description of Artificial Sequence: Synthetic probe
gaggttca

SEQ ID NO: 63 - DNA, 29 bp, Artificial Sequence
  Description of Artificial Sequence: Synthetic probe
aatgatacggcgaccaccgagatctacac

SEQ ID NO: 64 - DNA, 12 bp, Artificial Sequence
  Description of Artificial Sequence: Synthetic probe
  modified_base (1)..(12): a, c, g, or t
  misc_feature (1)..(12): This sequence may encompass 8-12 nucleotides
  See specification as filed for detailed description of substitutions and preferred embodiments
nnnnnnnnnnnn

SEQ ID NO: 65 - DNA, 14 bp, Artificial Sequence
  Description of Artificial Sequence: Synthetic probe
tcgtcggcagcgtc

SEQ ID NO: 66 - DNA, 24 bp, Artificial Sequence
  Description of Artificial Sequence: Synthetic probe
caagcagaagacggcatacgagat

SEQ ID NO: 67 - DNA, 12 bp, Artificial Sequence
  Description of Artificial Sequence: Synthetic probe
  modified_base (1)..(12): a, c, g, or t
  misc_feature (1)..(12): This sequence may encompass 8-12 nucleotides
  See specification as filed for detailed description of substitutions and preferred embodiments
nnnnnnnnnnnn

SEQ ID NO: 68 - DNA, 19 bp, Artificial Sequence
  Description of Artificial Sequence: Synthetic probe
gtgactggagttcagacgt

SEQ ID NO: 69 - DNA, 8 bp, Artificial Sequence
  Description of Artificial Sequence: Synthetic probe
caacgttc

SEQ ID NO: 70 - DNA, 30 bp, Artificial Sequence
  Description of Artificial Sequence: Synthetic probe
gaagcaggccatagagaagagggatcacat

SEQ ID NO: 71 - DNA, 8 bp, Artificial Sequence
  Description of Artificial Sequence: Synthetic probe
cagctgag

SEQ ID NO: 72 - DNA, 28 bp, Artificial Sequence
  Description of Artificial Sequence: Synthetic probe
tctaggaactacccggacctgtccttgg

SEQ ID NO: 73 - DNA, 8 bp, Artificial Sequence
  Description of Artificial Sequence: Synthetic probe
ctctctcg

SEQ ID NO: 74 - DNA, 24 bp, Artificial Sequence
  Description of Artificial Sequence: Synthetic probe
caccagcttgtctgcaggaggagg

SEQ ID NO: 75 - DNA, 8 bp, Artificial Sequence
  Description of Artificial Sequence: Synthetic probe
agagagat

SEQ ID NO: 76 - DNA, 24 bp, Artificial Sequence
  Description of Artificial Sequence: Synthetic probe
caccagcttgtctgcaggaggaga

SEQ ID NO: 77 - DNA, 8 bp, Artificial Sequence
  Description of Artificial Sequence: Synthetic probe
gagaagat

SEQ ID NO: 78 - DNA, 21 bp, Artificial Sequence
  Description of Artificial Sequence: Synthetic probe
ccgaggggaggccgtccacgt

SEQ ID NO: 79 - DNA, 8 bp, Artificial Sequence
  Description of Artificial Sequence: Synthetic probe
cacagcga

SEQ ID NO: 80 - DNA, 29 bp, Artificial Sequence
  Description of Artificial Sequence: Synthetic probe
gaattctctctcctcacctgcagcatcat

SEQ ID NO: 81 - DNA, 8 bp, Artificial Sequence
  Description of Artificial Sequence: Synthetic probe
tcaggata

SEQ ID NO: 82 - DNA, 26 bp, Artificial Sequence
  Description of Artificial Sequence: Synthetic probe
cagagctcccttcctgaccctccgcc

SEQ ID NO: 83 - DNA, 8 bp, Artificial Sequence
  Description of Artificial Sequence: Synthetic probe
tcggttac

SEQ ID NO: 84 - DNA, 30 bp, Artificial Sequence
  Description of Artificial Sequence: Synthetic probe
gaagcaggccatagagaagagggatcacaa

SEQ ID NO: 85 - DNA, 8 bp, Artificial Sequence
  Description of Artificial Sequence: Synthetic probe
tgcgtatc

SEQ ID NO: 86 - DNA, 28 bp, Artificial Sequence
  Description of Artificial Sequence: Synthetic probe
tctaggaactacccggacctgtccttga

SEQ ID NO: 87 - DNA, 8 bp, Artificial Sequence
  Description of Artificial Sequence: Synthetic probe
accggttc

SEQ ID NO: 88 - DNA, 24 bp, Artificial Sequence
  Description of Artificial Sequence: Synthetic probe
caccagcttgtctgcaggaggagc

SEQ ID NO: 89 - DNA, 8 bp, Artificial Sequence
  Description of Artificial Sequence: Synthetic probe
actgtgag

SEQ ID NO: 90 - DNA, 21 bp, Artificial Sequence
  Description of Artificial Sequence: Synthetic probe
ccgaggggaggccgtccacgc

SEQ ID NO: 91 - DNA, 8 bp, Artificial Sequence
  Description of Artificial Sequence: Synthetic probe
tctcctcg

SEQ ID NO: 92 - DNA, 29 bp, Artificial Sequence
  Description of Artificial Sequence: Synthetic probe
gaattctctctcctcacctgcagcatcaa

SEQ ID NO: 93 - skipped

SEQ ID NO: 94 - DNA, 26 bp, Artificial Sequence
  Description of Artificial Sequence: Synthetic probe
cagagctcccttcctgaccctccgct

SEQ ID NO: 95 - DNA, 27 bp, Artificial Sequence
  Description of Artificial Sequence: Synthetic probe
cgtggagatgcagctgaggcagcacaa

SEQ ID NO: 96 - DNA, 8 bp, Artificial Sequence
  Description of Artificial Sequence: Synthetic probe
gatagcat

SEQ ID NO: 97 - DNA, 31 bp, Artificial Sequence
  Description of Artificial Sequence: Synthetic probe
gagactactccctgctctggaaagcccacaa

SEQ ID NO: 98 - DNA, 8 bp, Artificial Sequence
  Description of Artificial Sequence: Synthetic probe
aaccaggt

SEQ ID NO: 99 - DNA, 22 bp, Artificial Sequence
  Description of Artificial Sequence: Synthetic probe
tgggggctggagggtgggaact

SEQ ID NO: 100 - DNA, 8 bp, Artificial Sequence
  Description of Artificial Sequence: Synthetic probe
catgacta

SEQ ID NO: 101 - DNA, 25 bp, Artificial Sequence
  Description of Artificial Sequence: Synthetic probe
acagtccccaccttgtgctgcctca

SEQ ID NO: 102 - DNA, 8 bp, Artificial Sequence
  Description of Artificial Sequence: Synthetic probe
gtctcgga

SEQ ID NO: 103 - DNA, 31 bp, Artificial Sequence
  Description of Artificial Sequence: Synthetic probe
ctgttacctcaccttcggagacaagatcaag

SEQ ID NO: 104 - DNA, 8 bp, Artificial Sequence
  Description of Artificial Sequence: Synthetic probe
ctctaagt

SEQ ID NO: 105 - DNA, 30 bp, Artificial Sequence
  Description of Artificial Sequence: Synthetic probe
gcagaggattgaggcttaattctgagctgg

SEQ ID NO: 106 - DNA, 8 bp, Artificial Sequence
  Description of Artificial Sequence: Synthetic probe
catcgtgt

SEQ ID NO: 107 - DNA, 8 bp, Artificial Sequence
  Description of Artificial Sequence: Synthetic probe
gcaacctt

SEQ ID NO: 108 - DNA, 8 bp, Artificial Sequence
  Description of Artificial Sequence: Synthetic probe
gagattct

SEQ ID NO: 109 - DNA, 8 bp, Artificial Sequence
  Description of Artificial Sequence: Synthetic probe
cactgctt

SEQ ID NO: 110 - DNA, 8 bp, Artificial Sequence
  Description of Artificial Sequence: Synthetic probe
aggtacga

SEQ ID NO: 111 - DNA, 8 bp, Artificial Sequence
  Description of Artificial Sequence: Synthetic probe
accgagtc

SEQ ID NO: 112 - DNA, 8 bp, Artificial Sequence
  Description of Artificial Sequence: Synthetic probe
cacaagta

SEQ ID NO: 113 - DNA, 8 bp, Artificial Sequence
  Description of Artificial Sequence: Synthetic probe
tcaaccac

SEQ ID NO: 114 - DNA, 25 bp, Artificial Sequence
  Description of Artificial Sequence: Synthetic probe
ccttccttctttttgattttgtcag

SEQ ID NO: 115 - DNA, 8 bp, Artificial Sequence
  Description of Artificial Sequence: Synthetic probe
agctctgc

SEQ ID NO: 116 - DNA, 25 bp, Artificial Sequence
  Description of Artificial Sequence: Synthetic probe
ccttccttctttttgattttgtcaa

SEQ ID NO: 117 - DNA, 8 bp, Artificial Sequence
  Description of Artificial Sequence: Synthetic probe
gtaccgtc

SEQ ID NO: 118 - DNA, 18 bp, Artificial Sequence
  Description of Artificial Sequence: Synthetic probe
ggcacacgtggcttttcg

SEQ ID NO: 119 - DNA, 8 bp, Artificial Sequence
  Description of Artificial Sequence: Synthetic probe
gtgcgcag

SEQ ID NO: 120 - DNA, 22 bp, Artificial Sequence
  Description of Artificial Sequence: Synthetic probe
agccgacactttgcttgctatg

SEQ ID NO: 121 - DNA, 8 bp, Artificial Sequence
  Description of Artificial Sequence: Synthetic probe
tccttccg

SEQ ID NO: 122 - DNA, 19 bp, Artificial Sequence
  Description of Artificial Sequence: Synthetic probe
agatttggacctgcgagcg

SEQ ID NO: 123 - DNA, 8 bp, Artificial Sequence
  Description of Artificial Sequence: Synthetic probe
attctcct

SEQ ID NO: 124 - DNA, 24 bp, Artificial Sequence
  Description of Artificial Sequence: Synthetic probe
ttgcataaacttacgaggttcacc

SEQ ID NO: 125 - DNA, 8 bp, Artificial Sequence
  Description of Artificial Sequence: Synthetic probe
tctgtgac

SEQ ID NO: 126 - DNA, 26 bp, Artificial Sequence
  Description of Artificial Sequence: Synthetic probe
cattctgttcttcaagcacctatgtc

SEQ ID NO: 127 - DNA, 8 bp, Artificial Sequence
  Description of Artificial Sequence: Synthetic probe
tcattgca

SEQ ID NO: 128 - DNA, 20 bp, Artificial Sequence
  Description of Artificial Sequence: Synthetic probe
acttgtggagacagccgctc

SEQ ID NO: 129 - DNA, 8 bp, Artificial Sequence
  Description of Artificial Sequence: Synthetic probe
gtggccag

SEQ ID NO: 130 - DNA, 21 bp, Artificial Sequence
  Description of Artificial Sequence: Synthetic probe
tccgctgaccatcaayaagga

SEQ ID NO: 131 - DNA, 26 bp, Artificial Sequence
  Description of Artificial Sequence: Synthetic probe
gaaaatatgtggttggagagctcatt

SEQ ID NO: 132 - DNA, 8 bp, Artificial Sequence
  Description of Artificial Sequence: Synthetic probe
ttgatata

SEQ ID NO: 133 - DNA, 18 bp, Artificial Sequence
  Description of Artificial Sequence: Synthetic probe
gagtatgcctgccgtgtg

SEQ ID NO: 134 - DNA, 8 bp, Artificial Sequence
  Description of Artificial Sequence: Synthetic probe
agcgatat

SEQ ID NO: 135 - DNA, 31 bp, Artificial Sequence
  Description of Artificial Sequence: Synthetic probe
tggagataacactctaagcataactaaaggt

SEQ ID NO: 136 - DNA, 21 bp, Artificial Sequence
  Description of Artificial Sequence: Synthetic probe
ttgagcctcagggtctgagtg

SEQ ID NO: 137 - DNA, 22 bp, Artificial Sequence
  Description of Artificial Sequence: Synthetic probe
taaaaaggggatcttcactcgg

SEQ ID NO: 138 - DNA, 18 bp, Artificial Sequence
  Description of Artificial Sequence: Synthetic probe
agatgccgcatttggatt

SEQ ID NO: 139 - DNA, 21 bp, Artificial Sequence
  Description of Artificial Sequence: Synthetic probe
tgggtcccaagcaactacatc

SEQ ID NO: 140 - DNA, 24 bp, Artificial Sequence
  Description of Artificial Sequence: Synthetic probe
acaagtctgttctacacaagtaca

SEQ ID NO: 141 - DNA, 19 bp, Artificial Sequence
  Description of Artificial Sequence: Synthetic probe
ggttagaggagacccctcc

SEQ ID NO: 142 - DNA, 17 bp, Artificial Sequence
  Description of Artificial Sequence: Synthetic probe
cgagttcccggcgatga

SEQ ID NO: 143 - DNA, 24 bp, Artificial Sequence
  Description of Artificial Sequence: Synthetic probe
acaatgtcgtcttacaccattgag

SEQ ID NO: 144 - DNA, 34 bp, Artificial Sequence
  Description of Artificial Sequence: Synthetic probe
gtgactggagttcagacgtgtgctcttccgatct

SEQ ID NO: 145 - DNA, 19 bp, Artificial Sequence
  Description of Artificial Sequence: Synthetic probe
ctcccgtgatcttctgcac

SEQ ID NO: 146 - DNA, 19 bp, Artificial Sequence
  Description of Artificial Sequence: Synthetic probe
tcccagcgtcaatatgctg

SEQ ID NO: 147 - DNA, 20 bp, Artificial Sequence
  Description of Artificial Sequence: Synthetic probe
ccaccgcactgaggaatgtc

SEQ ID NO: 148 - DNA, 24 bp, Artificial Sequence
  Description of Artificial Sequence: Synthetic probe
acagacaatggactcccttagtgg

SEQ ID NO: 149 - DNA, 20 bp, Artificial Sequence
  Description of Artificial Sequence: Synthetic probe
gagcggctgtctccacaagt

SEQ ID NO: 150 - DNA, 18 bp, Artificial Sequence
  Description of Artificial Sequence: Synthetic probe
ctggcccaacgatggcga

SEQ ID NO: 151 - DNA, 21 bp, Artificial Sequence
  Description of Artificial Sequence: Synthetic probe
ggaggaggtgggcatctaccg

SEQ ID NO: 152 - DNA, 19 bp, Artificial Sequence
  Description of Artificial Sequence: Synthetic probe
tcttcctgcccaacagcaa

SEQ ID NO: 153 - DNA, 19 bp, Artificial Sequence
  Description of Artificial Sequence: Synthetic probe
acctggatggaccgcctag

SEQ ID NO: 154 - DNA, 19 bp, Artificial Sequence
  Description of Artificial Sequence: Synthetic probe
ccgatggcttcgacgagtt

SEQ ID NO: 155 - DNA, 22 bp, Artificial Sequence
  Description of Artificial Sequence: Synthetic probe
cattagcacaacaggcctttga

SEQ ID NO: 156 - DNA, 22 bp, Artificial Sequence
  Description of Artificial Sequence: Synthetic probe
cacctaccacagagccatcaaa

SEQ ID NO: 157 - DNA, 19 bp, Artificial Sequence
  Description of Artificial Sequence: Synthetic probe
ccagcctcatgcacaacca

SEQ ID NO: 158 - DNA, 19 bp, Artificial Sequence
  Description of Artificial Sequence: Synthetic probe
ctctgtctccccgcctgaa

SEQ ID NO: 159 - DNA, 25 bp, Artificial Sequence
  Description of Artificial Sequence: Synthetic probe
cccaagtatccctgtaaaacaaaaa

SEQ ID NO: 160 - DNA, 23 bp, Artificial Sequence
  Description of Artificial Sequence: Synthetic probe
gatggagtccacaggatcagagt

SEQ ID NO: 161 - DNA, 21 bp, Artificial Sequence
  Description of Artificial Sequence: Synthetic probe
cactcagaccctgaggctcaa

SEQ ID NO: 162 - DNA, 21 bp, Artificial Sequence
  Description of Artificial Sequence: Synthetic probe
gcttgtagatgcggggtagag

SEQ ID NO: 163 - DNA, 17 bp, Artificial Sequence
  Description of Artificial Sequence: Synthetic probe
agggcccgcttggactt

SEQ ID NO: 164 - DNA, 21 bp, Artificial Sequence
  Description of Artificial Sequence: Synthetic probe
cctcgttaagcatccctgtga

SEQ ID NO: 165 - DNA, 23 bp, Artificial Sequence
  Description of Artificial Sequence: Synthetic probe
ctctttctccagcgtctgcttat

SEQ ID NO: 166 - DNA, 22 bp, Artificial Sequence
  Description of Artificial Sequence: Synthetic probe
atccacaggtgagtctggcatt

SEQ ID NO: 167 - DNA, 21 bp, Artificial Sequence
  Description of Artificial Sequence: Synthetic probe
gggctcctcggatactcaaaa

SEQ ID NO: 168 - DNA, 16 bp, Artificial Sequence
  Description of Artificial Sequence: Synthetic probe
cggctcgtgctggcat

SEQ ID NO: 169 - DNA, 22 bp, Artificial Sequence
  Description of Artificial Sequence: Synthetic probe
gaaaggaaacttggatggctca

SEQ ID NO: 170 - DNA, 22 bp, Artificial Sequence
  Description of Artificial Sequence: Synthetic probe
ccgagtgaagatccccttttta

SEQ ID NO: 171 - DNA, 18 bp, Artificial Sequence
  Description of Artificial Sequence: Synthetic probe
aatccaaatgcggcatct

SEQ ID NO: 172 - DNA, 21 bp, Artificial Sequence
  Description of Artificial Sequence: Synthetic probe
gatgtagttgcttgggaccca

SEQ ID NO: 173 - DNA, 24 bp, Artificial Sequence
  Description of Artificial Sequence: Synthetic probe
ggtgaacctcgtaagtttatgcaa

SEQ ID NO: 174 - DNA, 26 bp, Artificial Sequence
  Description of Artificial Sequence: Synthetic probe
gacataggtgcttgaagaacagaatg

SEQ ID NO: 175 - DNA, 55 bp, Artificial Sequence
  Description of Artificial Sequence: Synthetic probe
aatgatacggcgaccaccgagatctacacaacaacaacacctcgtcggcagcgtc

SEQ ID NO: 176 - DNA, 55 bp, Artificial Sequence
  Description of Artificial Sequence: Synthetic probe
aatgatacggcgaccaccgagatctacacaacaagtcctcgtcgtcggcagcgtc

SEQ ID NO: 177 - DNA, 55 bp, Artificial Sequence
  Description of Artificial Sequence: Synthetic probe
aatgatacggcgaccaccgagatctacacaacaccgattagtcgtcggcagcgtc

SEQ ID NO: 178 - DNA, 55 bp, Artificial Sequence
  Description of Artificial Sequence: Synthetic probe
aatgatacggcgaccaccgagatctacacaacaggagcgcatcgtcggcagcgtc

SEQ ID NO: 179 - DNA, 55 bp, Artificial Sequence
  Description of Artificial Sequence: Synthetic probe
aatgatacggcgaccaccgagatctacacaaccgagaactttcgtcggcagcgtc

SEQ ID NO: 180 - DNA, 55 bp, Artificial Sequence
  Description of Artificial Sequence: Synthetic probe
aatgatacggcgaccaccgagatctacacaacctgccacattcgtcggcagcgtc

SEQ ID NO: 181 - DNA, 55 bp, Artificial Sequence
  Description of Artificial Sequence: Synthetic probe
aatgatacggcgaccaccgagatctacacaactaattgcggtcgtcggcagcgtc

SEQ ID NO: 182 - DNA, 55 bp, Artificial Sequence
  Description of Artificial Sequence: Synthetic probe
aatgatacggcgaccaccgagatctacacaacttctcttcctcgtcggcagcgtc

SEQ ID NO: 183 - DNA, 55 bp, Artificial Sequence
  Description of Artificial Sequence: Synthetic probe
aatgatacggcgaccaccgagatctacacaagaaccgagcctcgtcggcagcgtc

SEQ ID NO: 184 - DNA, 55 bp, Artificial Sequence
  Description of Artificial Sequence: Synthetic probe
aatgatacggcgaccaccgagatctacacaagaaggttacgtcgtcggcagcgtc

SEQ ID NO: 185 - DNA, 55 bp, Artificial Sequence
  Description of Artificial Sequence: Synthetic probe
aatgatacggcgaccaccgagatctacacaagactatgacctcgtcggcagcgtc

SEQ ID NO: 186 - DNA, 55 bp, Artificial Sequence
  Description of Artificial Sequence: Synthetic probe
aatgatacggcgaccaccgagatctacacaagagtggaagttcgtcggcagcgtc

SEQ ID NO: 187 - DNA, 55 bp, Artificial Sequence
  Description of Artificial Sequence: Synthetic probe
aatgatacggcgaccaccgagatctacacaagcggtcattatcgtcggcagcgtc

SEQ ID NO: 188 - DNA, 55 bp, Artificial Sequence
  Description of Artificial Sequence: Synthetic probe
aatgatacggcgaccaccgagatctacacaaggtccggttgtcgtcggcagcgtc

SEQ ID NO: 189 - DNA, 55 bp, Artificial Sequence
  Description of Artificial Sequence: Synthetic probe
aatgatacggcgaccaccgagatctacacaatagcaatcggtcgtcggcagcgtc

SEQ ID NO: 190 - DNA, 55 bp, Artificial Sequence
  Description of Artificial Sequence: Synthetic probe
aatgatacggcgaccaccgagatctacacaatgcttactggtcgtcggcagcgtc

SEQ ID NO: 191 - DNA, 55 bp, Artificial Sequence
  Description of Artificial Sequence: Synthetic probe
aatgatacggcgaccaccgagatctacacaattacgagaggtcgtcggcagcgtc

SEQ ID NO: 192 - DNA, 55 bp, Artificial Sequence
  Description of Artificial Sequence: Synthetic probe
aatgatacggcgaccaccgagatctacacacaaccttcagctcgtcggcagcgtc

SEQ ID NO: 193 - DNA, 55 bp, Artificial Sequence
  Description of Artificial Sequence: Synthetic probe
aatgatacggcgaccaccgagatctacacacaatgacaaggtcgtcggcagcgtc

SEQ ID NO: 194 - DNA, 55 bp, Artificial Sequence
  Description of Artificial Sequence: Synthetic probe
aatgatacggcgaccaccgagatctacacacaggtaataggtcgtcggcagcgtc

SEQ ID NO: 195 - DNA, 55 bp, Artificial Sequence
  Description of Artificial Sequence: Synthetic probe
aatgatacggcgaccaccgagatctacacacattaacctcgtcgtcggcagcgtc

SEQ ID NO: 196 - DNA, 55 bp, Artificial Sequence
  Description of Artificial Sequence: Synthetic probe
aatgatacggcgaccaccgagatctacacaccgaacgccattcgtcggcagcgtc

SEQ ID NO: 197 - DNA, 55 bp, Artificial Sequence
  Description of Artificial Sequence: Synthetic probe
aatgatacggcgaccaccgagatctacacaccgtcagagtatcgtcggcagcgtc

SEQ ID NO: 198 - DNA, 55 bp, Artificial Sequence
  Description of Artificial Sequence: Synthetic probe
aatgatacggcgaccaccgagatctacacacgccatacatatcgtcggcagcgtc

SEQ ID NO: 199 - DNA, 55 bp, Artificial Sequence
  Description of Artificial Sequence: Synthetic probe
aatgatacggcgaccaccgagatctacacacgctgaagaattcgtcggcagcgtc

SEQ ID NO: 200 - DNA, 55 bp, Artificial Sequence
  Description of Artificial Sequence: Synthetic probe
aatgatacggcgaccaccgagatctacacacggttctaatctcgtcggcagcgtc

SEQ ID NO: 201 - DNA, 55 bp, Artificial Sequence
  Description of Artificial Sequence: Synthetic probe
aatgatacggcgaccaccgagatctacacactatcgcactttcgtcggcagcgtc

SEQ ID NO: 202 - DNA, 55 bp, Artificial Sequence
  Description of Artificial Sequence: Synthetic probe
aatgatacggcgaccaccgagatctacacagagcataaggatcgtcggcagcgtc

SEQ ID NO: 203 - DNA, 55 bp, Artificial Sequence
  Description of Artificial Sequence: Synthetic probe
aatgatacggcgaccaccgagatctacacagattccgccgttcgtcggcagcgtc

SEQ ID NO: 204 - DNA, 55 bp, Artificial Sequence
  Description of Artificial Sequence: Synthetic probe
aatgatacggcgaccaccgagatctacacaggaagagagagtcgtcggcagcgtc

SEQ ID NO: 205 - DNA, 55 bp, Artificial Sequence
  Description of Artificial Sequence: Synthetic probe
aatgatacggcgaccaccgagatctacacagtgtggttctctcgtcggcagcgtc

SEQ ID NO: 206 - DNA, 55 bp, Artificial Sequence
  Description of Artificial Sequence: Synthetic probe
aatgatacggcgaccaccgagatctacacataagactcacctcgtcggcagcgtc

SEQ ID NO: 207 - DNA, 55 bp, Artificial Sequence
  Description of Artificial Sequence: Synthetic probe
aatgatacggcgaccaccgagatctacacatcgtcgtgccttcgtcggcagcgtc

SEQ ID NO: 208 - DNA, 55 bp, Artificial Sequence
  Description of Artificial Sequence: Synthetic probe
aatgatacggcgaccaccgagatctacacatggagattggttcgtcggcagcgtc

SEQ ID NO: 209 - DNA, 55 bp, Artificial Sequence
  Description of Artificial Sequence: Synthetic probe
aatgatacggcgaccaccgagatctacacattcataccagctcgtcggcagcgtc

SEQ ID NO: 210 - DNA, 55 bp, Artificial Sequence
  Description of Artificial Sequence: Synthetic probe
aatgatacggcgaccaccgagatctacaccaatacgctgcatcgtcggcagcgtc

SEQ ID NO: 211 - DNA, 55 bp, Artificial Sequence
  Description of Artificial Sequence: Synthetic probe
aatgatacggcgaccaccgagatctacaccacctaactatctcgtcggcagcgtc

SEQ ID NO: 212 - DNA, 55 bp, Artificial Sequence
  Description of Artificial Sequence: Synthetic probe
aatgatacggcgaccaccgagatctacaccagagcaaccattcgtcggcagcgtc

SEQ ID NO: 213 - DNA, 55 bp, Artificial Sequence
  Description of Artificial Sequence: Synthetic probe
aatgatacggcgaccaccgagatctacaccagctcgccttatcgtcggcagcgtc

SEQ ID NO: 214 - DNA, 55 bp, Artificial Sequence
  Description of Artificial Sequence: Synthetic probe
aatgatacggcgaccaccgagatctacaccataatcagtcctcgtcggcagcgtc

SEQ ID NO: 215 - DNA, 55 bp, Artificial Sequence
  Description of Artificial Sequence: Synthetic probe
aatgatacggcgaccaccgagatctacaccatcaagaacggtcgtcggcagcgtc

SEQ ID NO: 216 - DNA, 55 bp, Artificial Sequence
  Description of Artificial Sequence: Synthetic probe
aatgatacggcgaccaccgagatctacaccatctaggttgttcgtcggcagcgtc

SEQ ID NO: 217 - DNA, 55 bp, Artificial Sequence
  Description of Artificial Sequence: Synthetic probe
aatgatacggcgaccaccgagatctacaccatgtgctattctcgtcggcagcgtc

SEQ ID NO: 218 - DNA, 55 bp, Artificial Sequence
  Description of Artificial Sequence: Synthetic probe
aatgatacggcgaccaccgagatctacaccatgtggaggaatcgtcggcagcgtc

SEQ ID NO: 219 - DNA, 55 bp, Artificial Sequence
  Description of Artificial Sequence: Synthetic probe
aatgatacggcgaccaccgagatctacacccaattctaccgtcgtcggcagcgtc

SEQ ID NO: 220 - DNA, 55 bp, Artificial Sequence
  Description of Artificial Sequence: Synthetic probe
aatgatacggcgaccaccgagatctacacccacaccacatatcgtcggcagcgtc

SEQ ID NO: 221 - DNA, 55 bp, Artificial Sequence
  Description of Artificial Sequence: Synthetic probe
aatgatacggcgaccaccgagatctacacccaccgcttctttcgtcggcagcgtc

SEQ ID NO: 222 - DNA, 55 bp, Artificial Sequence
  Description of Artificial Sequence: Synthetic probe
aatgatacggcgaccaccgagatctacacccatcttaatcgtcgtcggcagcgtc

SEQ ID NO: 223 - DNA, 55 bp, Artificial Sequence
  Description of Artificial Sequence: Synthetic probe
aatgatacggcgaccaccgagatctacacccgaatagaacttcgtcggcagcgtc

SEQ ID NO: 224 - DNA, 55 bp, Artificial Sequence
  Description of Artificial Sequence: Synthetic probe
aatgatacggcgaccaccgagatctacacccgcagtcctattcgtcggcagcgtc

SEQ ID NO: 225 - DNA, 55 bp, Artificial Sequence
  Description of Artificial Sequence: Synthetic probe
aatgatacggcgaccaccgagatctacacccggtgagttaatcgtcggcagcgtc

SEQ ID NO: 226 - DNA, 55 bp, Artificial Sequence
  Description of Artificial Sequence: Synthetic probe
aatgatacggcgaccaccgagatctacaccgtaagtgatggtcgtcggcagcgtc

SEQ ID NO: 227 - DNA, 55 bp, Artificial Sequence
  Description of Artificial Sequence: Synthetic probe
aatgatacggcgaccaccgagatctacacctaaccatgaagtcgtcggcagcgtc

SEQ ID NO: 228 - DNA, 55 bp, Artificial Sequence
  Description of Artificial Sequence: Synthetic probe
aatgatacggcgaccaccgagatctacacctagtgttcaagtcgtcggcagcgtc

SEQ ID NO: 229 - DNA, 55 bp, Artificial Sequence
  Description of Artificial Sequence: Synthetic probe
aatgatacggcgaccaccgagatctacacctccgatccaattcgtcggcagcgtc

SEQ ID NO: 230 - DNA, 55 bp, Artificial Sequence
  Description of Artificial Sequence: Synthetic probe
aatgatacggcgaccaccgagatctacacctgaactccgcatcgtcggcagcgtc

SEQ ID NO: 231 - DNA, 55 bp, Artificial Sequence
  Description of Artificial Sequence: Synthetic probe
aatgatacggcgaccaccgagatctacaccttacatgcctctcgtcggcagcgtc

SEQ ID NO: 232 - DNA, 55 bp, Artificial Sequence
  Description of Artificial Sequence: Synthetic probe
aatgatacggcgaccaccgagatctacacgaagtctccatttcgtcggcagcgtc

SEQ ID NO: 233 - DNA, 55 bp, Artificial Sequence
  Description of Artificial Sequence: Synthetic probe
aatgatacggcgaccaccgagatctacacgagccttagtcttcgtcggcagcgtc

SEQ ID NO: 234 - DNA, 55 bp, Artificial Sequence
  Description of Artificial Sequence: Synthetic probe
aatgatacggcgaccaccgagatctacacgaggaggtgttgtcgtcggcagcgtc

SEQ ID NO: 235 - DNA, 55 bp, Artificial Sequence
  Description of Artificial Sequence: Synthetic probe
aatgatacggcgaccaccgagatctacacgataaccgcatatcgtcggcagcgtc

SEQ ID NO: 236 - DNA, 55 bp, Artificial Sequence
  Description of Artificial Sequence: Synthetic probe
aatgatacggcgaccaccgagatctacacgatggactgaggtcgtcggcagcgtc

SEQ ID NO: 237 - DNA, 55 bp, Artificial Sequence
  Description of Artificial Sequence: Synthetic probe
aatgatacggcgaccaccgagatctacacgcagcaccgtaatcgtcggcagcgtc

SEQ ID NO: 238 - DNA, 55 bp, Artificial Sequence
  Description of Artificial Sequence: Synthetic probe
aatgatacggcgaccaccgagatctacacgcctataattcctcgtcggcagcgtc

SEQ ID NO: 239 - DNA, 55 bp, Artificial Sequence
  Description of Artificial Sequence: Synthetic probe
aatgatacggcgaccaccgagatctacacgctactcaccaatcgtcggcagcgtc

SEQ ID NO: 240 - DNA, 55 bp, Artificial Sequence
  Description of Artificial Sequence: Synthetic probe
aatgatacggcgaccaccgagatctacacgctgcaatatactcgtcggcagcgtc

SEQ ID NO: 241 - DNA, 55 bp, Artificial Sequence
  Description of Artificial Sequence: Synthetic probe
aatgatacggcgaccaccgagatctacacggtagatcattgtcgtcggcagcgtc

SEQ ID NO: 242 - DNA, 55 bp, Artificial Sequence
  Description of Artificial Sequence: Synthetic probe
aatgatacggcgaccaccgagatctacacgtactgttcctttcgtcggcagcgtc

SEQ ID NO: 243 - DNA, 55 bp, Artificial Sequence
  Description of Artificial Sequence: Synthetic probe
aatgatacggcgaccaccgagatctacacgtctccgtctcttcgtcggcagcgtc

SEQ ID NO: 244 - DNA, 55 bp, Artificial Sequence
  Description of Artificial Sequence: Synthetic probe
aatgatacggcgaccaccgagatctacacgtgttatgttggtcgtcggcagcgtc

SEQ ID NO: 245 - DNA, 55 bp, Artificial Sequence
  Description of Artificial Sequence: Synthetic probe
aatgatacggcgaccaccgagatctacacgttctcatagcttcgtcggcagcgtc

SEQ ID NO: 246 - DNA, 55 bp, Artificial Sequence
  Description of Artificial Sequence: Synthetic probe
aatgatacggcgaccaccgagatctacactagccacgttcctcgtcggcagcgtc

SEQ ID NO: 247 - DNA, 55 bp, Artificial Sequence
  Description of Artificial Sequence: Synthetic probe
aatgatacggcgaccaccgagatctacactagcttaacacctcgtcggcagcgtc

SEQ ID NO: 248 - DNA, 55 bp, Artificial Sequence
  Description of Artificial Sequence: Synthetic probe
aatgatacggcgaccaccgagatctacactagtgacgatggtcgtcggcagcgtc

SEQ ID NO: 249 - DNA, 55 bp, Artificial Sequence
  Description of Artificial Sequence: Synthetic probe
aatgatacggcgaccaccgagatctacactatagagcaaggtcgtcggcagcgtc

SEQ ID NO: 250 - DNA, 55 bp, Artificial Sequence
  Description of Artificial Sequence: Synthetic probe
aatgatacggcgaccaccgagatctacactatcattcgctctcgtcggcagcgtc

SEQ ID NO: 251 - DNA, 55 bp, Artificial Sequence
  Description of Artificial Sequence: Synthetic probe
aatgatacggcgaccaccgagatctacactccgtattagcctcgtcggcagcgtc

SEQ ID NO: 252 - DNA, 55 bp, Artificial Sequence
  Description of Artificial Sequence: Synthetic probe
aatgatacggcgaccaccgagatctacactgagagcctatttcgtcggcagcgtc

SEQ ID NO: 253 - DNA, 55 bp, Artificial Sequence
  Description of Artificial Sequence: Synthetic probe
aatgatacggcgaccaccgagatctacactggttggagtaatcgtcggcagcgtc

SEQ ID NO: 254 - DNA, 55 bp, Artificial Sequence
  Description of Artificial Sequence: Synthetic probe
aatgatacggcgaccaccgagatctacactgttgcttgatctcgtcggcagcgtc

SEQ ID NO: 255 - DNA, 55 bp, Artificial Sequence
  Description of Artificial Sequence: Synthetic probe
aatgatacggcgaccaccgagatctacacttaacggtcgagtcgtcggcagcgtc

SEQ ID NO: 256 - DNA, 55 bp, Artificial Sequence
  Description of Artificial Sequence: Synthetic probe
aatgatacggcgaccaccgagatctacacttaccaaccgaatcgtcggcagcgtc

SEQ ID NO: 257 - DNA, 55 bp, Artificial Sequence
  Description of Artificial Sequence: Synthetic probe
aatgatacggcgaccaccgagatctacacttatgtgctgcgtcgtcggcagcgtc

SEQ ID NO: 258 - DNA, 55 bp, Artificial Sequence
  Description of Artificial Sequence: Synthetic probe
aatgatacggcgaccaccgagatctacacttcctcacctcctcgtcggcagcgtc

SEQ ID NO: 259 - DNA, 55 bp, Artificial Sequence
  Description of Artificial Sequence: Synthetic probe
aatgatacggcgaccaccgagatctacacttggaagtacggtcgtcggcagcgtc

SEQ ID NO: 260 - DNA, 55 bp, Artificial Sequence
  Description of Artificial Sequence: Synthetic probe
caagcagaagacggcatacgagataacaacaacaccgtgactggagttcagacgt

SEQ ID NO: 261 - DNA, 55 bp, Artificial Sequence
  Description of Artificial Sequence: Synthetic probe
caagcagaagacggcatacgagataacaagtcctcggtgactggagttcagacgt

SEQ ID NO: 262 - DNA, 55 bp, Artificial Sequence
  Description of Artificial Sequence: Synthetic probe
caagcagaagacggcatacgagataacaccgattaggtgactggagttcagacgt

SEQ ID NO: 263 - DNA, 55 bp, Artificial Sequence
  Description of Artificial Sequence: Synthetic probe
caagcagaagacggcatacgagataacaggagcgcagtgactggagttcagacgt

SEQ ID NO: 264 - DNA, 55 bp, Artificial Sequence
  Description of Artificial Sequence: Synthetic probe
caagcagaagacggcatacgagataaccgagaacttgtgactggagttcagacgt

SEQ ID NO: 265 - DNA, 55 bp, Artificial Sequence
  Description of Artificial Sequence: Synthetic probe
caagcagaagacggcatacgagataacctgccacatgtgactggagttcagacgt

SEQ ID NO: 266 - DNA, 55 bp, Artificial Sequence
  Description of Artificial Sequence: Synthetic probe
caagcagaagacggcatacgagataactaattgcgggtgactggagttcagacgt

SEQ ID NO: 267 - DNA, 55 bp, Artificial Sequence
  Description of Artificial Sequence: Synthetic probe
caagcagaagacggcatacgagataacttctcttccgtgactggagttcagacgt

SEQ ID NO: 268 - DNA, 55 bp, Artificial Sequence
  Description of Artificial Sequence: Synthetic probe
caagcagaagacggcatacgagataagaaccgagccgtgactggagttcagacgt

SEQ ID NO: 269 - DNA, 55 bp, Artificial Sequence
  Description of Artificial Sequence: Synthetic probe
caagcagaagacggcatacgagataagaaggttacggtgactggagttcagacgt

SEQ ID NO: 270 - DNA, 55 bp, Artificial Sequence
  Description of Artificial Sequence: Synthetic probe
caagcagaagacggcatacgagataagactatgaccgtgactggagttcagacgt

SEQ ID NO: 271 - DNA, 55 bp, Artificial Sequence
  Description of Artificial Sequence: Synthetic probe
caagcagaagacggcatacgagataagagtggaagtgtgactggagttcagacgt

SEQ ID NO: 272 - DNA, 55 bp, Artificial Sequence
  Description of Artificial Sequence: Synthetic probe
caagcagaagacggcatacgagataagcggtcattagtgactggagttcagacgt

SEQ ID NO: 273 - DNA, 55 bp, Artificial Sequence
  Description of Artificial Sequence: Synthetic probe
caagcagaagacggcatacgagataaggtccggttggtgactggagttcagacgt

SEQ ID NO: 274 - DNA, 55 bp, Artificial Sequence
  Description of Artificial Sequence: Synthetic probe
caagcagaagacggcatacgagataatagcaatcgggtgactggagttcagacgt

SEQ ID NO: 275 - DNA, 55 bp, Artificial Sequence
  Description of Artificial Sequence: Synthetic probe
caagcagaagacggcatacgagataatgcttactgggtgactggagttcagacgt

SEQ ID NO: 276 - DNA, 55 bp, Artificial Sequence
  Description of Artificial Sequence: Synthetic probe
caagcagaagacggcatacgagataattacgagagggtgactggagttcagacgt

SEQ ID NO: 277 - DNA, 55 bp, Artificial Sequence
  Description of Artificial Sequence: Synthetic probe
caagcagaagacggcatacgagatacaaccttcagcgtgactggagttcagacgt

SEQ ID NO: 278 - DNA, 55 bp, Artificial Sequence
  Description of Artificial Sequence: Synthetic probe
caagcagaagacggcatacgagatacaatgacaagggtgactggagttcagacgt

SEQ ID NO: 279 - DNA, 55 bp, Artificial Sequence
  Description of Artificial Sequence: Synthetic probe
caagcagaagacggcatacgagatacaggtaatagggtgactggagttcagacgt

SEQ ID NO: 280 - DNA, 55 bp, Artificial Sequence
  Description of Artificial Sequence: Synthetic probe
caagcagaagacggcatacgagatacattaacctcggtgactggagttcagacgt

SEQ ID NO: 281 - DNA, 55 bp, Artificial Sequence
  Description of Artificial Sequence: Synthetic probe
caagcagaagacggcatacgagataccgaacgccatgtgactggagttcagacgt

SEQ ID NO: 282 - DNA, 55 bp, Artificial Sequence
  Description of Artificial Sequence: Synthetic probe
caagcagaagacggcatacgagataccgtcagagtagtgactggagttcagacgt

SEQ ID NO: 283 - DNA, 55 bp, Artificial Sequence
  Description of Artificial Sequence: Synthetic probe
caagcagaagacggcatacgagatacgccatacatagtgactggagttcagacgt

SEQ ID NO: 284 - DNA, 55 bp, Artificial Sequence
  Description of Artificial Sequence: Synthetic probe
caagcagaagacggcatacgagatacgctgaagaatgtgactggagttcagacgt

SEQ ID NO: 285 - DNA, 55 bp, Artificial Sequence
  Description of Artificial Sequence: Synthetic probe
caagcagaagacggcatacgagatacggttctaatcgtgactggagttcagacgt

SEQ ID NO: 286 - DNA, 55 bp, Artificial Sequence
  Description of Artificial Sequence: Synthetic probe
caagcagaagacggcatacgagatactatcgcacttgtgactggagttcagacgt

SEQ ID NO: 287 - DNA, 55 bp, Artificial Sequence
  Description of Artificial Sequence: Synthetic probe
caagcagaagacggcatacgagatagagcataaggagtgactggagttcagacgt

SEQ ID NO: 288 - DNA, 55 bp, Artificial Sequence
  Description of Artificial Sequence: Synthetic probe
caagcagaagacggcatacgagatagattccgccgtgtgactggagttcagacgt

SEQ ID NO: 289 - DNA, 55 bp, Artificial Sequence
  Description of Artificial Sequence: Synthetic probe
caagcagaagacggcatacgagataggaagagagaggtgactggagttcagacgt

SEQ ID NO: 290 - DNA, 55 bp, Artificial Sequence
  Description of Artificial Sequence: Synthetic probe
caagcagaagacggcatacgagatagtgtggttctcgtgactggagttcagacgt

SEQ ID NO: 291 - DNA, 55 bp, Artificial Sequence
  Description of Artificial Sequence: Synthetic probe
caagcagaagacggcatacgagatataagactcaccgtgactggagttcagacgt

SEQ ID NO: 292 - DNA, 55 bp, Artificial Sequence
  Description of Artificial Sequence: Synthetic probe
caagcagaagacggcatacgagatatcgtcgtgcctgtgactggagttcagacgt

SEQ ID NO: 293 - DNA, 55 bp, Artificial Sequence
  Description of Artificial Sequence: Synthetic probe
caagcagaagacggcatacgagatatggagattggtgtgactggagttcagacgt

SEQ ID NO: 294 - DNA, 55 bp, Artificial Sequence
  Description of Artificial Sequence: Synthetic probe
caagcagaagacggcatacgagatattcataccagcgtgactggagttcagacgt

SEQ ID NO: 295 - DNA, 55 bp, Artificial Sequence
  Description of Artificial Sequence: Synthetic probe
caagcagaagacggcatacgagatcaatacgctgcagtgactggagttcagacgt

SEQ ID NO: 296 - DNA, 55 bp, Artificial Sequence
  Description of Artificial Sequence: Synthetic probe
caagcagaagacggcatacgagatcacctaactatcgtgactggagttcagacgt

SEQ ID NO: 297 - DNA, 55 bp, Artificial Sequence
  Description of Artificial Sequence: Synthetic probe
caagcagaagacggcatacgagatcagagcaaccatgtgactggagttcagacgt

SEQ ID NO: 298 - DNA, 55 bp, Artificial Sequence
  Description of Artificial Sequence: Synthetic probe
caagcagaagacggcatacgagatcagctcgccttagtgactggagttcagacgt

SEQ ID NO: 299 - DNA, 55 bp, Artificial Sequence
  Description of Artificial Sequence: Synthetic probe
caagcagaagacggcatacgagatcataatcagtccgtgactggagttcagacgt

SEQ ID NO: 300 - DNA, 55 bp, Artificial Sequence
  Description of Artificial Sequence: Synthetic probe
caagcagaagacggcatacgagatcatcaagaacgggtgactggagttcagacgt

SEQ ID NO: 301 - DNA, 55 bp, Artificial Sequence
  Description of Artificial Sequence: Synthetic probe
caagcagaagacggcatacgagatcatctaggttgtgtgactggagttcagacgt

SEQ ID NO: 302 - DNA, 55 bp, Artificial Sequence
  Description of Artificial Sequence: Synthetic probe
caagcagaagacggcatacgagatcatgtgctattcgtgactggagttcagacgt

SEQ ID NO: 303 - DNA, 55 bp, Artificial Sequence
  Description of Artificial Sequence: Synthetic probe
caagcagaagacggcatacgagatcatgtggaggaagtgactggagttcagacgt

SEQ ID NO: 304 - DNA, 55 bp, Artificial Sequence
  Description of Artificial Sequence: Synthetic probe
caagcagaagacggcatacgagatccaattctaccggtgactggagttcagacgt

SEQ ID NO: 305 - DNA, 55 bp, Artificial Sequence
  Description of Artificial Sequence: Synthetic probe
caagcagaagacggcatacgagatccacaccacatagtgactggagttcagacgt

SEQ ID NO: 306 - DNA, 55 bp, Artificial Sequence
  Description of Artificial Sequence: Synthetic probe
caagcagaagacggcatacgagatccaccgcttcttgtgactggagttcagacgt

SEQ ID NO: 307 - DNA, 55 bp, Artificial Sequence
  Description of Artificial Sequence: Synthetic probe
caagcagaagacggcatacgagatccatcttaatcggtgactggagttcagacgt

SEQ ID NO: 308 - DNA, 55 bp, Artificial Sequence
  Description of Artificial Sequence: Synthetic probe
caagcagaagacggcatacgagatccgaatagaactgtgactggagttcagacgt

SEQ ID NO: 309 - DNA, 55 bp, Artificial Sequence
  Description of Artificial Sequence: Synthetic probe
caagcagaagacggcatacgagatccgcagtcctatgtgactggagttcagacgt

SEQ ID NO: 310 - DNA, 55 bp, Artificial Sequence
  Description of Artificial Sequence: Synthetic probe
caagcagaagacggcatacgagatccggtgagttaagtgactggagttcagacgt

SEQ ID NO: 311 - DNA, 55 bp, Artificial Sequence
  Description of Artificial Sequence: Synthetic probe
caagcagaagacggcatacgagatcgtaagtgatgggtgactggagttcagacgt

SEQ ID NO: 312 - DNA, 55 bp, Artificial Sequence
  Description of Artificial Sequence: Synthetic probe
caagcagaagacggcatacgagatctaaccatgaaggtgactggagttcagacgt

SEQ ID NO: 313 - DNA, 55 bp, Artificial Sequence
  Description of Artificial Sequence: Synthetic probe
caagcagaagacggcatacgagatctagtgttcaaggtgactggagttcagacgt

SEQ ID NO: 314 - DNA, 55 bp, Artificial Sequence
  Description of Artificial Sequence: Synthetic probe
caagcagaagacggcatacgagatctccgatccaatgtgactggagttcagacgt

SEQ ID NO: 315 - DNA, 55 bp, Artificial Sequence
  Description of Artificial Sequence: Synthetic probe
caagcagaagacggcatacgagatctgaactccgcagtgactggagttcagacgt

SEQ ID NO: 316 - DNA, 55 bp, Artificial Sequence
  Description of Artificial Sequence: Synthetic probe
caagcagaagacggcatacgagatcttacatgcctcgtgactggagttcagacgt

SEQ ID NO: 317 - DNA, 55 bp, Artificial Sequence
  Description of Artificial Sequence: Synthetic probe
caagcagaagacggcatacgagatgaagtctccattgtgactggagttcagacgt

SEQ ID NO: 318 - DNA, 55 bp, Artificial Sequence
  Description of Artificial Sequence: Synthetic probe
caagcagaagacggcatacgagatgagccttagtctgtgactggagttcagacgt

SEQ ID NO: 319 - DNA, 55 bp, Artificial Sequence
  Description of Artificial Sequence: Synthetic probe
caagcagaagacggcatacgagatgaggaggtgttggtgactggagttcagacgt

SEQ ID NO: 320 - DNA, 55 bp, Artificial Sequence
  Description of Artificial Sequence: Synthetic probe
caagcagaagacggcatacgagatgataaccgcatagtgactggagttcagacgt

SEQ ID NO: 321 - DNA, 55 bp, Artificial Sequence
  Description of Artificial Sequence: Synthetic probe
caagcagaagacggcatacgagatgatggactgagggtgactggagttcagacgt

SEQ ID NO: 322 - DNA, 55 bp, Artificial Sequence
  Description of Artificial Sequence: Synthetic probe
caagcagaagacggcatacgagatgcagcaccgtaagtgactggagttcagacgt

SEQ ID NO: 323 - DNA, 55 bp, Artificial Sequence
  Description of Artificial Sequence: Synthetic probe
caagcagaagacggcatacgagatgcctataattccgtgactggagttcagacgt

SEQ ID NO: 324 - DNA, 55 bp, Artificial Sequence
  Description of Artificial Sequence: Synthetic probe
caagcagaagacggcatacgagatgctactcaccaagtgactggagttcagacgt

SEQ ID NO: 325 - DNA, 55 bp, Artificial Sequence
  Description of Artificial Sequence: Synthetic probe
caagcagaagacggcatacgagatgctgcaatatacgtgactggagttcagacgt

SEQ ID NO: 326 - DNA, 55 bp, Artificial Sequence
  Description of Artificial Sequence: Synthetic probe
caagcagaagacggcatacgagatggtagatcattggtgactggagttcagacgt

SEQ ID NO: 327 - DNA, 55 bp, Artificial Sequence
  Description of Artificial Sequence: Synthetic probe
caagcagaagacggcatacgagatgtactgttccttgtgactggagttcagacgt

SEQ ID NO: 328 - DNA, 55 bp, Artificial Sequence
  Description of Artificial Sequence: Synthetic probe
caagcagaagacggcatacgagatgtctccgtctctgtgactggagttcagacgt

SEQ ID NO: 329 - DNA, 55 bp, Artificial Sequence
  Description of Artificial Sequence: Synthetic probe
caagcagaagacggcatacgagatgtgttatgttgggtgactggagttcagacgt

SEQ ID NO: 330 - DNA, 55 bp, Artificial Sequence
  Description of Artificial Sequence: Synthetic probe
caagcagaagacggcatacgagatgttctcatagctgtgactggagttcagacgt

SEQ ID NO: 331 - DNA, 55 bp, Artificial Sequence
  Description of Artificial Sequence: Synthetic probe
caagcagaagacggcatacgagattagccacgttccgtgactggagttcagacgt

SEQ ID NO: 332 - DNA, 55 bp, Artificial Sequence
  Description of Artificial Sequence: Synthetic probe
caagcagaagacggcatacgagattagcttaacaccgtgactggagttcagacgt

SEQ ID NO: 333 - DNA, 55 bp, Artificial Sequence
  Description of Artificial Sequence: Synthetic probe
caagcagaagacggcatacgagattagtgacgatgggtgactggagttcagacgt

SEQ ID NO: 334 - DNA, 55 bp, Artificial Sequence
  Description of Artificial Sequence: Synthetic probe
caagcagaagacggcatacgagattatagagcaagggtgactggagttcagacgt

SEQ ID NO: 335 - DNA, 55 bp, Artificial Sequence
  Description of Artificial Sequence: Synthetic probe
caagcagaagacggcatacgagattatcattcgctcgtgactggagttcagacgt

SEQ ID NO: 336 - DNA, 55 bp, Artificial Sequence
  Description of Artificial Sequence: Synthetic probe
caagcagaagacggcatacgagattccgtattagccgtgactggagttcagacgt

SEQ ID NO: 337 - DNA, 55 bp, Artificial Sequence
  Description of Artificial Sequence: Synthetic probe
caagcagaagacggcatacgagattgagagcctattgtgactggagttcagacgt

SEQ ID NO: 338 - DNA, 55 bp, Artificial Sequence
  Description of Artificial Sequence: Synthetic probe
caagcagaagacggcatacgagattggttggagtaagtgactggagttcagacgt

SEQ ID NO: 339 - DNA, 55 bp, Artificial Sequence
  Description of Artificial Sequence: Synthetic probe
caagcagaagacggcatacgagattgttgcttgatcgtgactggagttcagacgt

SEQ ID NO: 340 - DNA, 55 bp, Artificial Sequence
  Description of Artificial Sequence: Synthetic probe
caagcagaagacggcatacgagatttaacggtcgaggtgactggagttcagacgt

SEQ ID NO: 341 - DNA, 55 bp, Artificial Sequence
  Description of Artificial Sequence: Synthetic probe
caagcagaagacggcatacgagatttaccaaccgaagtgactggagttcagacgt

SEQ ID NO: 342 - DNA, 55 bp, Artificial Sequence
  Description of Artificial Sequence: Synthetic probe
caagcagaagacggcatacgagatttatgtgctgcggtgactggagttcagacgt

SEQ ID NO: 343 - DNA, 55 bp, Artificial Sequence
  Description of Artificial Sequence: Synthetic probe
caagcagaagacggcatacgagatttcctcacctccgtgactggagttcagacgt

SEQ ID NO: 344 - DNA, 55 bp, Artificial Sequence
  Description of Artificial Sequence: Synthetic probe
caagcagaagacggcatacgagatttggaagtacgggtgactggagttcagacgt